(19) United States
(12) Patent Application Publication
(10) Pub. No.: US 2025/0063946 A1
(43) Pub. Date: Feb. 20, 2025

(54) PHOTOELECTRIC CONVERSION ELEMENT, IMAGING ELEMENT, OPTICAL SENSOR, AND COMPOUND

(71) Applicant: FUJIFILM Corporation, Tokyo (JP)

(72) Inventors: Yuko SUZUKI, Kanagawa (JP); Hiroki Sugirua, Kanagawa (JP)

(73) Assignee: FUJIFILM Corporation, Tokyo (JP)

(21) Appl. No.: 18/939,925

(22) Filed: Nov. 7, 2024

Related U.S. Application Data
(63) Continuation of application No. PCT/JP2023/017158, filed on May 2, 2023.

(30) Foreign Application Priority Data
May 10, 2022 (JP) 2022-077715

(51) Int. Cl. H10K 85/60; C07D 333/22; C07D 333/28; C07D 409/14; H10K 30/30

(52) U.S. Cl. CPC H10K 85/655; C07D 333/22; C07D 333/28; C07D 409/14; H10K 30/30

(57) ABSTRACT

In the present invention, there is provided a photoelectric conversion element which has excellent quantum efficiency and responsiveness in a case of receiving blue light, and has low electric field strength dependence of response speed. In addition, there is provided an imaging element, a photo sensor, an optical sensor, and a compound related to the photoelectric conversion element. The photoelectric conversion element according to the present invention is a photoelectric conversion element including a conductive film, a photoelectric conversion film, and a transparent conductive film in this order, in which the photoelectric conversion film contains a compound represented by Formula (1).

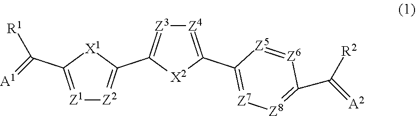

### CROSS-REFERENCE TO RELATED APPLICATIONS

This application is a Continuation of PCT International Application No. PCT/JP2023/017158 filed on May 2, 2023, which claims priority under 35 U.S.C. § 119(a) to Japanese Patent Application No. 2022-077715 filed on May 10, 2022. The above applications are hereby expressly incorporated by reference, in their entirety, into the present application.

### BACKGROUND OF THE INVENTION

### 1. Field of the Invention

The present invention relates to a photoelectric conversion element, an imaging element, an optical sensor, and a compound.

### 2. Description of the Related Art

In recent years, the development of an element (for example, an imaging element) having a photoelectric conversion film has been progressing.

For example, JP2014-026244A discloses a photoelectric conversion element containing a specific compound as an electron accepting material.

### SUMMARY OF THE INVENTION

In recent years, along with the demand for improving the performance of imaging elements, optical sensors, and the like, further improvements are required for various characteristics required for photoelectric conversion elements used therein. For example, it is required at a higher level that a quantum efficiency in a case where the photoelectric conversion element receives blue light (particularly, a wavelength of 460 nm) is high, a response speed in a case where the photoelectric conversion element receives blue light is excellent (hereinafter, also simply referred to as “excellent in responsiveness”), and an electric field strength dependence of the response speed is low. Here, the above-described blue light refers to light in a wavelength range of 400 to 500 nm, and “the electric field strength dependence of the response speed is low” refers to a small change in the response speed in a case where the voltage applied to the photoelectric conversion element is changed.

As a result of studying the photoelectric conversion element containing the compound disclosed in JP2014-026244A, the present inventors have found that there is still room for further improvement even in any of the quantum efficiency, the responsiveness, and the electric field strength dependence of the response speed in the case of receiving the blue light.

An object of the present invention is to provide a photoelectric conversion element which has excellent quantum efficiency and responsiveness in a case of receiving blue light, and has low electric field strength dependence of response speed.

Another object of the present invention is to provide an imaging element, an optical sensor, and a compound related to the above-described photoelectric conversion element.

In order to achieve the above objects, the inventors of the present invention carried out intensive examinations. As a result, the inventors have found that the objects can be achieved by the following constitution.

[1]A photoelectric conversion element comprising, in the following order, a conductive film, a photoelectric conversion film, and a transparent conductive film, in which the photoelectric conversion film contains a compound represented by Formula (1).

[2] The photoelectric conversion element according to [1], in which the compound represented by Formula (1) includes a compound represented by any of Formulae (1-1) to (1-7).

[3] The photoelectric conversion element according to [2], in which Rs is selected from the group consisting of a linear aliphatic hydrocarbon group having 1 to 3 carbon atoms, which may have a halogen atom, a branched aliphatic hydrocarbon group having 3 to 5 carbon atoms, which may have a halogen atom, a cyclic aliphatic hydrocarbon group having 3 to 8 carbon atoms, which may have a halogen atom, an aromatic ring group having 4 to 10 carbon atoms, which may have a substituent, and an alkoxy group having 1 to 3 carbon atoms.

[4] The photoelectric conversion element according to any one of [1] to [3], in which A¹ and A² are each a group represented by Formula (A-1).

[5] The photoelectric conversion element according to any one of [1] to [4], in which the group represented by Formula (A-1) is a group represented by Formula (C-1) or a group represented by Formula (C-2).

[6] The photoelectric conversion element according to any one of [1] to [5], in which X¹ and X² are each independently a sulfur atom or an oxygen atom.

[7] The photoelectric conversion element according to any one of [1] to [6], in which X¹ and X² are each a sulfur atom.

[8] The photoelectric conversion element according to any one of [1] to [7], in which the photoelectric conversion film further contains an n-type organic semiconductor, and the photoelectric conversion film has a bulk hetero structure formed in a state where the compound represented by Formula (1) and the n-type organic semiconductor are mixed.

[9] The photoelectric conversion element according to [8], in which the n-type organic semiconductor includes fullerenes selected from the group consisting of a fullerene and a derivative of the fullerene.

[10] The photoelectric conversion element according to any one of [1] to [9], in which the photoelectric conversion film further contains a coloring agent.

[11] The photoelectric conversion element according to any one of [1] to [10], in which the photoelectric conversion film further contains a p-type organic semiconductor.

[12] The photoelectric conversion element according to any one of [1] to [11], further comprising one or more interlayers between the conductive film and the transparent conductive film, in addition to the photoelectric conversion film.

[13] An imaging element comprising the photoelectric conversion element according to any one of [1] to [12].

[14] An optical sensor comprising the photoelectric conversion element according to any one of [1] to [12].

[15]A compound represented by Formula (1).

[16] The compound according to [15], in which the compound represented by Formula (1) includes a compound represented by any of Formulae (1-1) to (1-7).

[17] The compound according to [16], in which Rs is selected from the group consisting of a linear aliphatic hydrocarbon group having 1 to 3 carbon atoms, which may have a halogen atom, a branched aliphatic hydrocarbon group having 3 to 5 carbon atoms, which may have a halogen atom, a cyclic aliphatic hydrocarbon group having 3 to 8 carbon atoms, which may have a halogen atom, an aromatic ring group having 4 to 10 carbon atoms, which may have a substituent, and an alkoxy group having 1 to 3 carbon atoms.

[18] The compound according to any one of [15] to [17], in which A¹ and A² are each a group represented by Formula (A-1).

[19] The compound according to any one of [15] to [18], in which the group represented by Formula (A-1) is a group represented by Formula (C-1) or a group represented by Formula (C-2).

[20] The compound according to any one of [15] to [19], in which X¹ and X² are each independently a sulfur atom or an oxygen atom.

[21] The compound according to any one of [15] to [20], in which X¹ and X² are each a sulfur atom.

According to the present invention, it is possible to provide a photoelectric conversion element which has excellent quantum efficiency and response speed in a case of receiving blue light, and has low electric field strength dependence of response speed.

In addition, according to the present invention, it is possible to provide the imaging element, the optical sensor, and the compound related to the photoelectric conversion element.

### BRIEF DESCRIPTION OF THE DRAWINGS

FIG. 1 is a schematic cross-sectional view illustrating a configuration example of a photoelectric conversion element.

FIG. 2 is a schematic cross-sectional view illustrating a configuration example of the photoelectric conversion element.

### DESCRIPTION OF THE PREFERRED EMBODIMENTS

Hereinafter, the present invention will be described in detail.

Description of configuration requirements described below may be made on the basis of representative embodiments of the present invention in some cases, but the present invention is not limited to such embodiments.

Hereinafter, embodiments of the photoelectric conversion element according to the present invention will be described in detail.

In the present specification, numerical ranges represented by “to” include numerical values before and after “to” as lower limit values and upper limit values.

In the present specification, a hydrogen atom may be a light hydrogen atom (an ordinary hydrogen atom) or a deuterium atom (for example, a double hydrogen atom and the like).

In the present specification, in a case where there are plural substituents, linking groups, and the like (hereinafter, referred to as “substituents and the like”) represented by specific symbols, or a case where a plurality of substituents and the like are specified all together, each of the substituents and the like may be the same or may be different from each other. This also applies to a case of specifying the number of substituents and the like.

In the present specification, a “substituent” includes a group exemplified by a substituent W described later, unless otherwise specified.

### (Substituent W)

A substituent W in the present specification will be described below.

Examples of the substituent W include a halogen atom (for example, a fluorine atom, a chlorine atom, a bromine atom, an iodine atom, and the like), an alkyl group (including a cycloalkyl group, a bicycloalkyl group, and a tricycloalkyl group), an alkenyl group (including a cycloalkenyl group and a bicycloalkenyl group), an alkynyl group, an aryl group, a heteroaryl group (a heterocyclic group), a cyano group, a nitro group, an alkoxy group, an aryloxy group, a silyl group, a silyloxy group, a heterocyclic oxy group, an acyloxy group, a carbamoyloxy group, an alkoxycarbonyloxy group, an aryloxycarbonyloxy group, a secondary or tertiary amino group (including an anilino group), an alkylthio group, an arylthio group, a heterocyclic thio group, an alkyl or an arylsulfinyl group, an alkyl or an arylsulfonyl group, an acyl group, an aryloxycarbonyl group, an alkoxycarbonyl group, an aryl or a heterocyclic azo group, an imide group, a phosphino group, a phosphinyl group, a phosphinyloxy group, a phosphinylamino group, a phosphono group, a carboxy group, a phosphoric acid group, a sulfonic acid group, a hydroxy group, a thiol group, an acylamino group, a carbamoyl group, a ureido group, a boronic acid group, and a primary amino group. Each of the above-described groups may further have a substituent (for example, one or more groups of each of the above-described groups, and the like), as possible. For example, an alkyl group which may have a substituent is also included as a form of the substituent W.

In addition, in a case where the substituent W has a carbon atom, the number of carbon atoms of the substituent W is, for example, 1 to 20.

The number of atoms other than a hydrogen atom included in the substituent W is, for example, 1 to 30.

In addition, the specific compound described later preferably does not contain, as a substituent, a carboxy group, a salt of a carboxy group, a salt of a phosphoric acid group, a sulfonic acid group, a salt of a sulfonic acid group, a hydroxy group, a thiol group, an acylamino group, a carbamoyl group, a ureido group, or a boronic acid group (—B(OH)₂) and/or a primary amino group.

In the present specification, the aliphatic hydrocarbon group may be linear, branched, or cyclic.

Examples of the above-described aliphatic hydrocarbon group include an alkyl group, an alkenyl group, and an alkynyl group.

In the present specification, examples of the halogen atom include a fluorine atom, a chlorine atom, a bromine atom, and an iodine atom.

In the present specification, unless otherwise specified, the number of carbon atoms of the alkyl group is preferably 1 to 20, more preferably 1 to 10, and still more preferably 1 to 6.

The alkyl group may be any of linear, branched, or cyclic.

Examples of the alkyl group include a methyl group, an ethyl group, a n-propyl group, an i-propyl group, a n-butyl group, a t-butyl group, a n-hexyl group, a cyclopentyl group, and the like.

In addition, the alkyl group may be any of a cycloalkyl group, a bicycloalkyl group, or a tricycloalkyl group, and may have a ring structure thereof as a partial structure.

In the alkyl group which may have a substituent, examples of a substituent which may be contained in the alkyl group include the substituent W, and an aryl group (preferably having 6 to 18 carbon atoms, and more preferably having 6 carbon atoms), a heteroaryl group (preferably having 5 to 18 carbon atoms, and more preferably having 5 and 6 carbon atoms), or a halogen atom (preferably a fluorine atom or a chlorine atom) is preferable.

In the present specification, unless otherwise specified, the above-described alkyl group is preferable as an alkyl group moiety in the alkoxy group. The alkyl group moiety in the alkylthio group is preferably the above-described alkyl group.

In the alkoxy group which may have a substituent, the substituent which may be contained in the alkoxy group includes the same examples as the substituent in the alkyl group which may have a substituent. In the alkylthio group which may have a substituent, the substituent which may be contained in the alkylthio group includes the same examples as the substituent in the alkyl group which may have a substituent.

In the present specification, the alkenyl group may be any of linear, branched, or cyclic, unless otherwise specified. The number of carbon atoms of the alkenyl group is preferably 2 to 20. In the alkenyl group which may have a substituent, the substituent which may be contained in the alkenyl group includes the same examples as the substituent in the alkyl group which may have a substituent.

In the present specification, an alkynyl group may be any of linear, branched, or cyclic, unless otherwise specified. The number of carbon atoms of the alkynyl group is preferably 2 to 20. In the alkynyl group which may have a substituent, the substituent which may be contained in the alkynyl group includes the same examples as the substituent in the alkyl group which may have a substituent.

In the present specification, an aromatic ring constituting the aromatic ring structure or the aromatic ring group may be any of a monocyclic ring or a polycyclic ring (for example, 2 to 6 rings or the like), unless otherwise specified. The monocyclic aromatic ring is an aromatic ring having only one aromatic ring structure as a ring structure. The polycyclic (for example, 2 to 6 rings or the like) aromatic ring is an aromatic ring formed by a plurality of (for example, 2 to 6 or the like) aromatic ring structures being fused, as a ring structure.

The number of ring member atoms of the above-described aromatic ring is preferably 4 to 15.

The aromatic ring may be an aromatic hydrocarbon ring or an aromatic heterocyclic ring.

In a case where the aromatic ring is an aromatic heterocyclic ring, the number of heteroatoms contained as ring member atoms is, for example, 1 to 10. Examples of the heteroatoms include a nitrogen atom, a sulfur atom, an oxygen atom, a selenium atom, a tellurium atom, a phosphorus atom, a silicon atom, and a boron atom.

Examples of the aromatic hydrocarbon ring include a benzene ring, a naphthalene ring, an anthracene ring, and a phenanthrene ring.

Examples of the aromatic heterocyclic ring include a pyridine ring, a pyrimidine ring, a pyridazine ring, a pyrazine ring, a triazine ring (for example, 1,2,3-triazine ring, 1,2,4-triazine ring, 1,3,5-triazine ring, and the like), a tetrazine ring (for example, 1,2,4,5-tetrazine ring and the like), a quinoxaline ring, a pyrrole ring, a furan ring, a thiophene ring, an imidazole ring, an oxazole ring, a thiazole ring, a benzopyrrole ring, a benzofuran ring, a benzothiophene ring, a benzimidazole ring, a benzoxazole ring, a benzothiazole ring, a naphthopyrrole ring, a naphthofuran ring, a naphthothiophene ring, a naphthimidazole ring, a naphthoxazole ring, a 3H-pyrrolidine ring, a pyrroloimidazole ring (for example, a 5H-pyrrolo[1,2-a]imidazole ring and the like), an imidazooxazole ring (for example, an imidazo[2,1-b]oxazole ring and the like), a thienothiazole ring (for example, a thieno[2,3-d]thiazole ring and the like), a benzothiadiazole ring, a benzodithiophene ring (for example, benzo[1,2-b:4,5-b′]dithiophene ring and the like), a thienothiophene ring (for example, thieno[3,2-b]thiophene ring and the like), a thiazolothiazole ring (for example, thiazolo[5,4-d]thiazole ring and the like), a naphthodithiophene ring (for example, a naphtho[2,3-b:6,7-b′]dithiophene ring, a naphtho[2,1-b:6,5-b′]dithiophene ring, a naphtho[1,2-b:5,6-b′]dithiophene ring, a 1,8-dithiadicyclopenta[b,g]naphthalene ring, and the like), a benzothienobenzothiophene ring, a dithieno[3,2-b:2′,3′-d]thiophene ring, and a 3,4,7,8-tetrathiadicyclopenta[a,e]pentalene ring.

In the aromatic ring which may have a substituent, examples of the type of the substituent which may be contained in the aromatic ring include a group exemplified by the substituent W. In a case where the aromatic ring has substituents, the number of substituents may be 1 or more (for example, 1 to 4 or the like).

In the present specification, the term “aromatic ring group” includes, for example, a group obtained by removing one or more hydrogen atoms (for example, 1 to 5 or the like) from the aromatic ring.

In the present specification, the term “aryl group” includes, for example, a group obtained by removing one hydrogen atom from a ring corresponding to an aromatic hydrocarbon ring among the above aromatic rings.

In the present specification, the term “heteroaryl group” includes, for example, a group obtained by removing one hydrogen atom from a ring corresponding to an aromatic heterocyclic ring among the above aromatic rings.

In the present specification, the term “arylene group” includes, for example, a group obtained by removing two hydrogen atoms from a ring corresponding to an aromatic hydrocarbon ring among the above aromatic rings.

In the present specification, the term “heteroarylene group” includes, for example, a group obtained by removing two hydrogen atoms from a ring corresponding to an aromatic heterocyclic ring among the above aromatic rings.

In an aromatic ring group which may have a substituent, an aryl group which may have a substituent, a heteroaryl group which may have a substituent, an arylene group which may have a substituent, and a heteroarylene group which may have a substituent, examples of a type of the substituents that these groups may have include a group exemplified by the substituent W. In a case where these groups each of which may have a substituent have substituents, the number of substituents may be 1 or more (for example, 1 to 4 or the like).

In the present specification, in a case where a plurality of identical symbols indicating a type or the number of groups are present in Formula, which indicates a chemical structure, contents of these plurality of identical symbols indicating a type or the number of groups are independent of each other, and the contents of the identical symbols may be the same or different from each other unless otherwise specified.

In the present specification, in a case where a plurality of identical groups (for example, alkyl groups and the like) are present in one Formula, which indicates a chemical structure, specific contents between these pluralities of identical groups are independent of each other, and the specific contents between the plurality of identical groups may be the same or different from each other, unless otherwise specified.

The bonding direction of the divalent group (for example, —CO—O— and the like) denoted in the present specification, is not limited unless otherwise specified. For example, in a case where Y in a compound represented by a formula “X—Y—Z” is —CO—O—, the compound may be any of “X—O—CO—Z” or “X—CO—O—Z”.

In the present specification, regarding a compound that may have a geometric isomer (cis-trans isomer), a general formula or a structural formula representing the above compound may be described only in the form of either a cis isomer or a trans isomer for convenience. Even in such a case, unless otherwise specified, the form of the compound is not limited to either the cis isomer or the trans isomer, and the compound may be either the cis isomer or the trans isomer.

### [Photoelectric Conversion Element]

The photoelectric conversion element according to an embodiment of the present invention is a photoelectric conversion element including in the following order, a conductive film, a photoelectric conversion film, and a transparent conductive film, in which the photoelectric conversion film contains a compound represented by Formula (1) (hereinafter, referred to as a “specific compound”).

A mechanism by which the object of the present invention can be achieved by adopting the above-described configuration in the photoelectric conversion element according to the embodiment of the present invention is not always clear, but is presumed by the present inventors as follows.

The compound disclosed in JP2014-026244A has a symmetrical structure in which thiophene-benzene-thiophene are linked in this order by a single bond as a donor site, and thus has a property of being easily aggregated by π-π stacking. In the photoelectric conversion film, since charge separation in the photoelectric conversion film is not efficiently performed in a case where the aggregation of the compounds as described above occurs, the quantum efficiency, the response speed, and the like are reduced.

On the other hand, since the above-described specific compound has a partial structure in which two aromatic heterocyclic rings such as a thiophene ring having a donor property are linked by a single bond and further aromatic rings such as a benzene ring linked by a single bond, the donor site has an unsymmetrical structure as a whole such as thiophene-thiophene-benzene. It is presumed that, in such specific compounds, since it is considered that aggregation is unlikely to occur because of the unsymmetrical structure, efficient charge separation can be achieved, and the quantum efficiency, the response speed, and the like of the photoelectric conversion element are improved.

Hereinafter, obtaining at least one or more effects of more excellent quantum efficiency, more excellent responsiveness, or lower electric field strength dependence of the response speed is also referred to as being more excellent in the effect of the present invention.

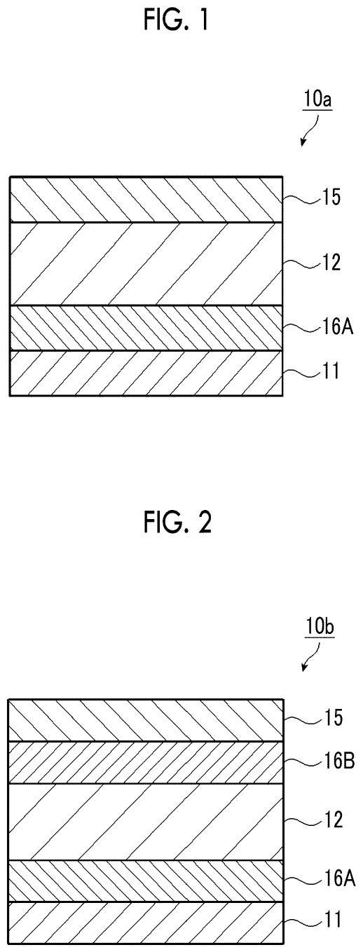

FIG. 1 is a schematic cross-sectional view of one embodiment of a photoelectric conversion element according to the embodiment of the present invention.

A photoelectric conversion element 10a illustrated in FIG. 1 has a configuration in which a conductive film (hereinafter, also referred to as a “lower electrode”) 11 functioning as a lower electrode, an electron blocking film 16A, a photoelectric conversion film 12 containing the specific compound, and a transparent conductive film (hereinafter, also referred to as an “upper electrode”) 15 functioning as an upper electrode are laminated in this order.

FIG. 2 illustrates a configuration example of another photoelectric conversion element. A photoelectric conversion element 10b illustrated in FIG. 2 has a configuration in which the electron blocking film 16A, the photoelectric conversion film 12, a positive hole blocking film 16B, and the upper electrode 15 are laminated on the lower electrode 11 in this order. The lamination order of the electron blocking film 16A, the photoelectric conversion film 12, and the positive hole blocking film 16B in FIGS. 1 and 2 may be appropriately changed according to the application and the characteristics.

In the photoelectric conversion element 10a (or 10b), it is preferable that light is incident on the photoelectric conversion film 12 through the upper electrode 15.

In a case where the photoelectric conversion element 10a (or 10b) is used, a voltage can be applied. In this case, it is preferable that the lower electrode 11 and the upper electrode 15 form a pair of electrodes, and a voltage of 1×10⁻⁵ to 1×10⁷ V/cm is applied between the pair of electrodes. From the viewpoint of the performance and power consumption, the applied voltage is more preferably 1×10⁴ to 1×10⁷ V/cm, and still more preferably 1×10³ to 5×10⁶ V/cm.

Regarding a voltage application method, in FIGS. 1 and 2, it is preferable that the voltage is applied such that the electron blocking film 16A side is a cathode and the photoelectric conversion film 12 side is an anode. In a case where the photoelectric conversion element 10a (or 10b) is used as an optical sensor, or also in a case where the photoelectric conversion element 10a (or 10b) is incorporated in an imaging element, the voltage can be applied by the same method.

As described in detail below, the photoelectric conversion element 10a (or 10b) can be suitably applied to applications of the imaging element.

Hereinafter, the form of each layer constituting the photoelectric conversion element according to the embodiment of the present invention will be described in detail.

### [Photoelectric Conversion Film]

The photoelectric conversion element has a photoelectric conversion film.

### <Specific Compound>

The photoelectric conversion film contains a compound (specific Compound) represented by Formula (1). In Formulae (A-1) and (A-2), * represents a bonding position.

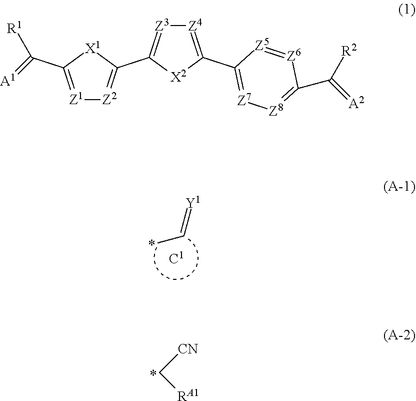

In Formula (1), R¹ and R² each independently represent a hydrogen atom or a substituent.

Examples of the substituent represented by R¹ and R² include a group exemplified by the above-described substituent W. Among these, from the viewpoint of being more excellent in the effect of the present invention, it is preferable that R¹ and R² are each a hydrogen atom.

In Formula (1), X¹ and X² each independently represent a sulfur atom, an oxygen atom, or a selenium atom.

Among these, from the viewpoint of being more excellent in the effect of the present invention, it is preferable that at least one of X¹ or X² is a sulfur atom or an oxygen atom, it is more preferable that X¹ and X² are each independently a sulfur atom or an oxygen atom, and it is still more preferable that both X¹ and X² are a sulfur atom.

In Formula (1), Z¹ to Z⁸ each independently represent —CR^{X1}═ or a nitrogen atom. R^{X1} represents a hydrogen atom or a substituent.

It is preferable that at least one of Z¹, . . . , or Z⁸ represents —CR^{X1}=. Among these, it is more preferable that at least one of Z², Z³, Z⁴, or Z⁵ represents —CR^{X1}=. In addition, two or more of Z¹ to Z⁸ may represent —CR^{X1}═.

In a case where two or more of Z¹ to Z⁸ represent —CR^{X1}═ and a plurality of R^{X1}'s are present, R^{X1}'s may be the same or different from each other.

Among these, from the viewpoint of being more excellent in the effect of the present invention, it is preferable that any one or two of Z¹ to Z⁸ (preferably, any one or two of Z², Z³, Z⁴, and Z⁵) are —CR^{X1}═ in which R^{X1} is a substituent, and the remaining Z¹ to Z⁸ are —CR^{X1}═ in which R^{X1} is a hydrogen atom, or a nitrogen atom.

Examples of the substituent represented by R^{X1} include a group exemplified by the above-described substituent W.

More specific examples of the substituent represented by R^{X1} include an aliphatic hydrocarbon group which may have a substituent, an aromatic ring group which may have a substituent, a halogen atom, an aliphatic heterocyclic group which may have a substituent, an alkoxy group which may have a substituent, an alkyloxycarbonyl group which may have a substituent, an alkylcarbonyl group which may have a substituent, —Si(R)₃, and —C—C—Si(R)₃. Among these, from the viewpoint of being more excellent in the effect of the present invention, an aliphatic hydrocarbon group which may have a substituent, an aromatic ring group which may have a substituent, or an alkoxy group which may have a substituent is preferable.

R represents a methyl group or an ethyl group, and R's may be the same or different from each other.

Examples of the substituent which may be included in the aliphatic hydrocarbon group, the aromatic ring group, the aliphatic heterocyclic group, the alkoxy group, the alkyloxycarbonyl group, and the alkylcarbonyl group, which are described above, include a group exemplified by the substituent W.

The number of carbon atoms of the above-described aliphatic hydrocarbon group is not particularly limited, and is preferably 1 to 20.

Examples of the aliphatic hydrocarbon group include a linear aliphatic hydrocarbon group, a branched aliphatic hydrocarbon group, and a cyclic aliphatic hydrocarbon group.

The number of carbon atoms of the linear aliphatic hydrocarbon group is not particularly limited, and is preferably 1 to 12, more preferably 1 to 5, and still more preferably 1 to 3.

The number of carbon atoms of the branched aliphatic hydrocarbon group is not particularly limited, and is preferably 3 to 10 and more preferably 3 to 5.

The number of carbon atoms of the cyclic aliphatic hydrocarbon group is not particularly limited, and is preferably 3 to 10 and more preferably 3 to 8.

As the substituent which may be included in the linear aliphatic hydrocarbon group, the branched aliphatic hydrocarbon group, and the cyclic aliphatic hydrocarbon group, a halogen atom is preferable.

The definition of the aromatic ring constituting the above-described aromatic ring group is as described above, and examples of the aromatic ring group include an aryl group and a heteroaryl group.

The number of carbon atoms of the aromatic ring group is not particularly limited, and is preferably 4 to 20 and more preferably 4 to 10.

The above-described aryl group is preferably a phenyl group which may have a substituent.

The above-described heteroaryl group is preferably a pyridine ring group which may have a substituent.

As the substituent which may be included in the aromatic ring group, an alkyl group is preferable.

In a case where the aromatic ring group has a substituent, the number of substituents is not particularly limited, and the aromatic ring group may have one substituent or a plurality of substituents.

The number of carbon atoms of the above-described alkoxy group is not particularly limited, and is preferably 1 to 20, more preferably 1 to 10, still more preferably 1 to 6, and particularly preferably 1 to 3.

As the alkoxy group, an alkoxy group formed by bonding an oxygen atom to the terminal on the bonding site side of each the aliphatic hydrocarbon group of the above-described preferred aspects is preferable, and a methoxy group or an ethoxy group is more preferable.

In a case where adjacent two of Z¹ to Z⁸ are —CR^{X1}═, two R^{X1}'s may be bonded to each other to form a ring.

Examples of the two adjacent Z¹ to Z⁸ include a combination of Z¹ and Z², a combination of Z³ and Z⁴, a combination of Z⁵ and Z⁶, and a combination of Z⁷ and Z⁸.

The ring formed by the two R^{X1}'s being bonded to each other may be an aromatic ring or an aliphatic ring.

In Formula (1), A¹ and A² each independently represent a group represented by Formula (A-1) or a group represented by Formula (A-2).

Among these, from the viewpoint of being more excellent in the effect of the present invention, it is preferable that at least one of A¹ or A² is the group represented by Formula (A-1), and it is more preferable that both A¹ and A² are the group represented by Formula (A-1).

In Formula (A-1), Y¹ represents a sulfur atom, an oxygen atom, =NR^{X2}, or =CR^{X3}R^{X4}. R^{X2} represents a hydrogen atom or a substituent. R^{X3} and R^{X4} each independently represent a cyano group, —SO₂R^{X5}, —COOR^{X6}, or —COR^{X7}.

From the viewpoint of being more excellent in the effect of the present invention, Y¹ is preferably an oxygen atom. In addition, examples of the substituent represented by R^{X2} include the substituents exemplified by the above-described substituent W.

R^{X5} to R^{X7} each independently represent an aliphatic hydrocarbon group which may have a substituent, an aromatic ring group which may have a substituent, or an aliphatic heterocyclic group which may have a substituent.

The definition of the aliphatic hydrocarbon group is as described above, and among these, an alkyl group is preferable, and a linear alkyl group is more preferable. The number of carbon atoms of the above-described alkyl group is preferably 1 to 3.

The definition of the aromatic ring group is as described above. The aromatic ring group may be any of an aryl group or a heteroaryl group. Among the above, a phenyl group is preferable.

The number of ring members in the aliphatic heterocyclic group is preferably 5 to 20, more preferably 5 to 12, and still more preferably 6 to 8.

Examples of the heteroatom which is contained in the aliphatic heterocyclic group include a sulfur atom, an oxygen atom, a nitrogen atom, a selenium atom, a tellurium atom, a phosphorus atom, a silicon atom, and a boron atom, and a sulfur atom, an oxygen atom, or a nitrogen atom is preferable.

Examples of the aliphatic heterocyclic ring constituting of the above-described aliphatic heterocyclic group include a pyrrolidine ring, an oxolane ring, a thiolane ring, a piperidine ring, a tetrahydrofuran ring, a tetrahydropyran ring, a thiane ring, a piperazine ring, a morpholine ring, a quinuclidine ring, a pyrrolidine ring, an azetidine ring, an oxetane ring, an aziridine ring, a dioxane ring, a pentamethylene sulfide ring, and γ-butyrolactone ring.

Examples of the substituent which may be included in the aliphatic hydrocarbon group, the aromatic ring group, and the aliphatic heterocyclic group include a group exemplified by the above-described substituent W.

In Formula (A-1), C¹ represents a ring which contains two or more carbon atoms and may have a substituent.

The number of carbon atoms of the ring is preferably 3 to 30, more preferably 3 to 20, and still more preferably 3 to 10. The number of the carbon atoms is a number containing two carbon atoms specified in the formula.

The ring may be any of aromatic or non-aromatic.

The ring may be any of a monocyclic ring or a polycyclic ring, and is preferably a 5-membered ring, a 6-membered ring, or a fused ring including at least one of a 5-membered ring or a 6-membered ring. The number of rings forming the fused ring is preferably 1 to 4, and more preferably 1 to 3.

The ring may have a heteroatom. Examples of the heteroatom include a nitrogen atom, a sulfur atom, an oxygen atom, a selenium atom, a tellurium atom, a phosphorus atom, a silicon atom, and a boron atom, and a sulfur atom, a nitrogen atom, or an oxygen atom is preferable. The number of heteroatoms in the ring is preferably 0 to 10 and more preferably 0 to 5.

Carbon atoms constituting the ring represented by C¹ may be substituted with another carbonyl carbon (>C═O) and/or another thiocarbonyl carbon (>C═S). The other carbonyl carbon (>C═O) and the other thiocarbonyl carbon (>C═S) mean a carbonyl carbon and a thiocarbonyl carbon each of which has a carbon atom other than the carbon atom at * part and the carbon atom bonded to Y¹ among the carbon atoms constituting the ring, as a constituent.

Examples of the substituent which may be contained in the ring include a group exemplified by the above-described substituent W, and a halogen atom, an alkyl group, an aromatic ring group, or a silyl group is preferable and a halogen atom or an alkyl group is more preferable.

The alkyl group may be linear, branched, or cyclic, and is preferably linear.

The number of carbon atoms of the above-described alkyl group is preferably 1 to 10 and more preferably 1 to 3.

As the ring represented by C¹, a ring which is used as an acidic nucleus (for example, an acidic nucleus of a merocyanine coloring agent) is preferable, and examples thereof include the following nuclei.
(a) 1,3-dicarbonyl nuclei: for example, a 1,3-indandione nucleus, 1,3-cyclohexanedione, 5,5-dimethyl-1,3-cyclohexanedione, 1,3-dioxane-4,6-dione, and the like;
(b) pyrazolinone nuclei: for example, 1-phenyl-2-pyrazolin-5-one, 3-methyl-1-phenyl-2-pyrazolin-5-one, 1-(2-benzothiazolyl)-3-methyl-2-pyrazolin-5-one, and the like;
(c) isoxazolinone nuclei: for example, 3-phenyl-2-isoxazolin-5-one, 3-methyl-2-isoxazolin-5-one, and the like;
(d) oxindole nuclei: for example, 1-alkyl-2,3-dihydro-2-oxindole and the like;
(e) 2,4,6-trioxohexahydropyrimidine nuclei: for example, barbituric acid, 2-thibarbituric acid and derivatives thereof, and the like, and examples of the derivatives include 1-alkyl compounds such as 1-methyl and 1-ethyl, 1,3-dialkyl compounds such as 1,3-dimethyl, 1,3-diethyl, and 1,3-dibutyl, 1,3-diaryl compounds such as 1,3-diphenyl, 1,3-di(p-chlorophenyl), and 1,3-di(p-ethoxycarbonylphenyl), 1-alkyl-1-aryl compounds such as 1-ethyl-3-phenyl, and 1,3-diheteroaryl compounds such as 1,3-di(2-pyridyl);
(f) 2-thio-2,4-thiazolidinedione nuclei: for example, rhodanine and derivatives thereof, and the like, and examples of the derivatives include 3-aklylrhodanine such as 3-methylrhodanine, 3-ethylrhodanine, and 3-allylrhodanine, 3-arylrhodanine such as 3-phenylrhodanine, 3-heteroarylrhodanine such as 3-(2-pyridyl)rhodanine, and the like;
(g) 2-thio-2,4-oxazolidinedione nuclei (2-thio-2,4-(3H,5H)-oxazoledione nuclei): for example, 3-ethyl-2-thio-2,4-oxazolidinedione, and the like;
(h) thianaphthenone nuclei: for example, 3(2H)-thianaphthenone-1,1-dioxide, and the like;
(i) 2-thio-2,5-thiazolidinedione nuclei: for example, 3-ethyl-2-thio-2,5-thiazolidinedione, and the like;
(j) 2,4-thiazolidinedione nuclei: for example, 2,4-thiazolidinedione, 3-ethyl-2,4-thiazolidinedione, 3-phenyl-2,4-thiazolidinedione, and the like;
(k) thiazoliin-4-one nuclei: for example, 4-thiazolinone, 2-ethyl-4-thiazolinone, and the like;
(l) 2,4-imidazolidinedione (hydantoin) nuclei: for example, 2,4-imidazolidinedione, 3-ethyl-2,4-imidazolidinedione, and the like;
(m) 2-thio-2,4-imidazolidinedione (2-thiohydantoin) nuclei: for example, 2-thio-2,4-imidazolidinedione, 3-ethyl-2-thio-2,4-imidazolidinedione, and the like;
(n) imidazolin-5-one nuclei: for example, 2-propylmercapto-2-imidazolin-5-one, and the like;
(o) 3,5-pyrazolidinedione nuclei: for example, 1,2-diphenyl-3,5-pyrazolidinedione, 1,2-dimethyl-3,5-pyrazolidinedione, and the like;
(p) benzothiophen-3(2H)-one nuclei: for example, benzothiophen-3(2H)-one, oxobenzothiophen-3(2H)-one, dioxobenzothiophen-3(2H)-one, and the like;
(q) indanone nuclei: for example, 1-indanone, 3-phenyl-1-indanone, 3-methyl-1-indanone, 3,3-diphenyl-1-indanone, 3,3-dimethyl-1-indanone, and the like;
(r) benzofuran-3-(2H)-one nucleus: for example, benzofuran-3-(2H)-one, and the like; and
(s) 2,2-dihydrophenalene-1,3-dione nucleus, and the like.

In a case where at least one of A¹ or A² in Formula (1) is the group represented by Formula (A-1), from the viewpoint of being more excellent in the effect of the present invention, the group represented by Formula (A-1) is preferably the group represented by Formula (C-1) or the group represented by Formula (C-2), and more preferably the group represented by Formula (C-2). In Formulae (C-1) and (C-2), * represents a bonding position.

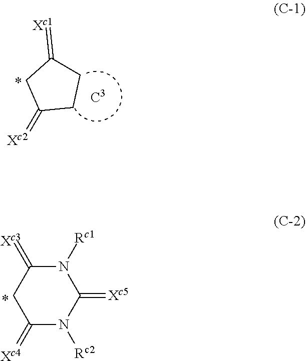

In Formula (C-1), X^{c1} and X^{c2} each independently represent a sulfur atom, an oxygen atom, ═NR^{Z1}, or ═CR^{Z2}R^{Z3}.

It is preferable that at least one of X^{c1} or X^{c2} is an oxygen atom, and it is more preferable that both X^{c1} and X^{c2} are oxygen atoms.

R^{Z1} represents a hydrogen atom or a substituent. Examples of the substituent represented by R^{Z1} include a group exemplified by the above-described substituent W.

R^{Z2} and R^{Z3} each independently represent a cyano group, —SO₂R^{Z4}, —COOR^{Z5}, or —COR^{Z6}.

Among these, it is preferable that at least one of R^{Z2} or R^{Z3} is a cyano group, and it is more preferable that both R^{Z2} and R^{Z3} are cyano groups.

R^{Z4} to R^{Z6} each independently represent an aliphatic hydrocarbon group which may have a substituent, an aromatic ring group which may have a substituent, or an aliphatic heterocyclic group which may have a substituent.

The definition of each group represented by R^{Z4} to R^{Z6} is the same as the definition of each group represented by R^{X5} to R^{X7}.

Examples of the substituent which may be included in the aliphatic hydrocarbon group, the aromatic ring group, and the aliphatic heterocyclic group include a group exemplified by the above-described substituent W.

In Formula (C-1), C³ represents an aromatic ring which may have a substituent.

The number of carbon atoms of the aromatic ring group is preferably 5 to 30, more preferably 5 to 12, and still more preferably 6 to 8. The number of the carbon atoms is a number containing two carbon atoms specified in the formula.

The aromatic ring group may be any of a monocyclic ring or a polycyclic ring.

In addition, the aromatic ring may be any of an aromatic hydrocarbon ring or an aromatic heterocyclic ring, and an aromatic hydrocarbon ring is preferable.

Examples of the aromatic ring represented by C³ include the ring exemplified in the description of the above-described aromatic ring.

Among these, from the viewpoint of being more excellent in the effect of the present invention, the above-described aromatic ring is preferably a benzene ring, a naphthalene ring, an anthracene ring, or a pyrene ring, and more preferably a benzene ring.

Examples of the substituent which may be included in the above-described aromatic ring include the group exemplified by the above-described substituent W.

In Formula (C-2), X^{c3} to X^{c5} each independently represent a sulfur atom, an oxygen atom, ═NR^{V1}, or ═CR^{V2}R^{V3}.

X^{c3} to X^{c5} are preferably an oxygen atom.

R^{V1} represents a hydrogen atom or a substituent. Examples of the substituent represented by R^{V1} include the group exemplified by the above-described substituent W.

In addition, R^{V2} and R^{V3} each independently represent a cyano group, —SO₂R^{V4}, —COOR^{V5}, or —COR^{V6}. R^{V4} to R^{V6} each independently represent an aliphatic hydrocarbon group which may have a substituent, an aromatic ring group which may have a substituent, or an aliphatic heterocyclic group which may have a substituent.

The aspects in which ═NR^{V1} and ═CR^{V2}R^{V3} may be adopted and suitable aspects thereof are the same as the aspects in which the above-described ═NR^{Z1} and the above-described ═CR^{Z2}R^{Z3} may be adopted and suitable aspects thereof.

In Formula (C-2), R^{c1} and R^{c2} each independently represent a hydrogen atom or a substituent. Examples of the substituent represented by R^{c1} and R^{c2} include the group exemplified by the above-described substituent W, and among these, an alkyl group or a phenyl group is preferable, and an alkyl group is more preferable.

The above-described phenyl group may further have a substituent, and examples thereof include a group exemplified by the above-described substituent W.

In Formula (A-2), R^{A1} represents a cyano group or —CO—Ar^{A}. Ar^{A} represents an aromatic ring group which may have a substituent or an aliphatic hydrocarbon group which may have a substituent.

Among these, from the viewpoint of being more excellent in the effect of the present invention, R^{A1} is preferably a cyano group or —CO—Ar^{A} which is an aromatic ring group where Ar^{A} may have a substituent.

The aromatic ring group represented by Ar^{A}, which may have a substituent, may be any of an aryl group or a heteroaryl group.

Among these, the aromatic ring group represented by Ar^{A}, which may have a substituent, is preferably a phenyl group which may have a substituent.

The aliphatic hydrocarbon group represented by Ar^{A}, which may have a substituent, may be linear, branched, or cyclic. The number of carbon atoms of the above-described aliphatic hydrocarbon group is preferably 1 to 30, more preferably 1 to 5, and still more preferably 1 to 3.

In Formula (1), in a case where A¹ and A² are each a group represented by Formula (A-1), the specific compound is represented by Formula (1A-1), and in a case where A¹ and A² are each a group represented by Formula (A-2), the specific compound is represented by Formula (1A-2).

In addition, in a case where the group represented by Formula (A-1) is a group represented by Formula (C-1), the specific compound is represented by Formula (1C-1), and in a case where the group represented by Formula (A-1) is a group represented by Formula (C-2), the specific compound is represented by Formula (1C-2).

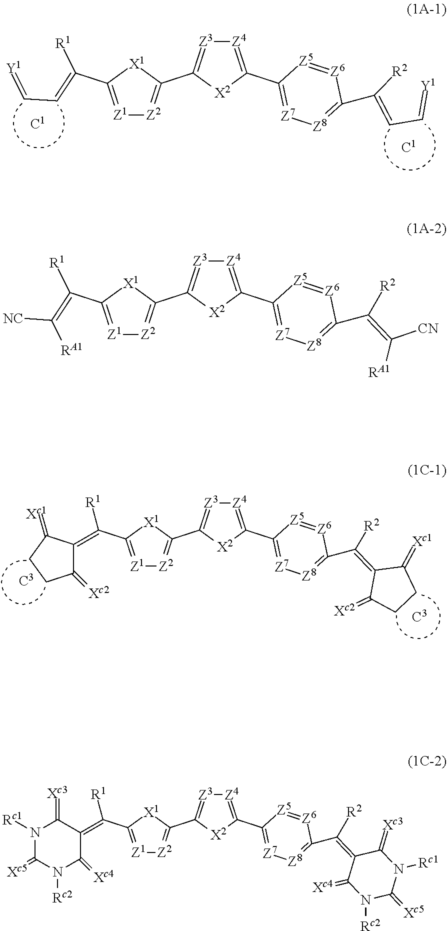

From the viewpoint of being more excellent in the effect of the present invention, the above-described specific compound is preferably a compound represented by any of Formulae (i-1) to (i-7), more preferably a compound represented by Formula (i-1), Formula (i-2), Formula (i-5), or Formula (i-6), and still more preferably a compound represented by Formula (i-1) or Formula (i-2).

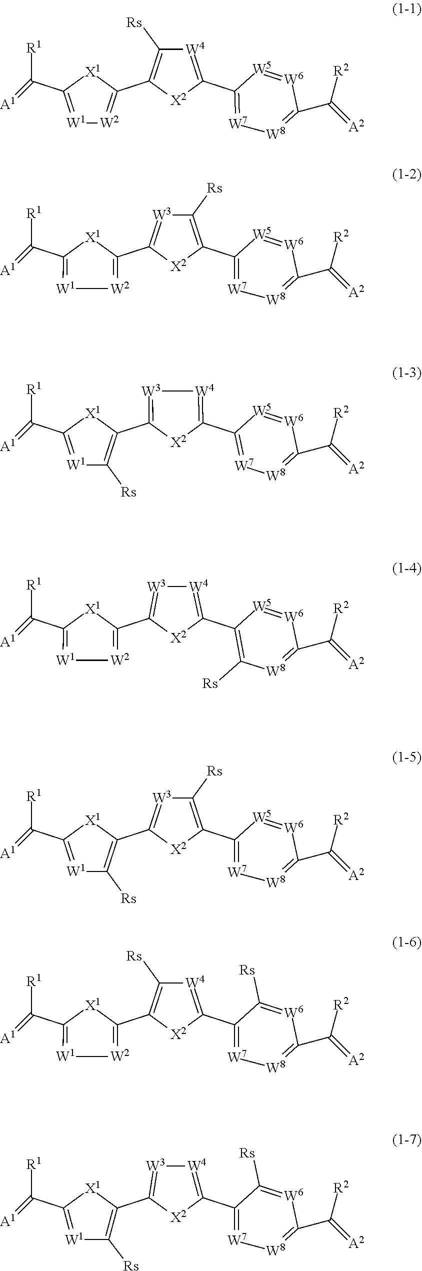

In Formulae (1-1) to (1-7), R¹ and R² each independently represent a hydrogen atom or a substituent. The definition and suitable aspects of the substituent represented by R¹ and R² are as described in Formula (1).

In Formula (1-1) to Formula (1-7), X¹ and X² each independently represent a sulfur atom, an oxygen atom, or a selenium atom.

Suitable aspects of X¹ and X² are as described in Formula (1).

In Formula (1-1) to Formula (1-7), A¹ and A² each independently represent the group represented by Formula (A-1) or Formula (A-2).

Suitable aspects of A¹ and A² are as described in Formula (1).

Specific aspects of Formula (A-1) and Formula (A-2) and preferred aspects thereof are as described above.

In Formula (1-1) to Formula (1-7), W¹ to W⁸ each independently represent —CR^{X1}═, and R^{X1} represents a hydrogen atom, a fluorine atom, or a chlorine atom.

In Formula (1-1), it is preferable that at least six of W¹, W², and W⁴ to W⁸ are —CR^{X1}═ in which R^{X1} is a hydrogen atom, and it is more preferable that all of W¹, W², and W⁴ to W⁸ are —CR^{X1}═ in which R^{X1} is a hydrogen atom.

In Formula (1-2), it is preferable that at least six of W¹ to W³, and W⁵ to W¹ are —CR^{X1}═ in which R^{X1} is a hydrogen atom, and it is more preferable that all of W¹ to W³, and W⁵ to W⁸ are —CR^{X1}═ in which R^{X1} is a hydrogen atom.

In Formula (1-3), it is preferable that at least six of W¹, and W³ to W¹ are —CR^{X1}═ in which R^{X1} is a hydrogen atom, and it is more preferable that all of W¹, and W³ to W⁸ are —CR^{X1}═ in which RX is a hydrogen atom.

In Formula (1-4), it is preferable that at least six of W¹ to W⁶, and W⁸ are —CR^{X1}═ in which RX is a hydrogen atom, and it is more preferable that all of W¹ to W⁶, and W⁸ are —CR^{X1}═ in which R^{X1} is a hydrogen atom.

In Formula (1-5), it is preferable that at least five of W¹, W³, and W⁵ to W⁸ are —CR^{X1}═ in which R^{X1} is a hydrogen atom, and it is more preferable that all of W¹, W³, and W⁵ to W⁸ are —CR^{X1}═ in which R^{X1} is a hydrogen atom.

In Formula (1-6), it is preferable that at least five of W¹, W², W⁴, and W⁶ to W¹ are —CR^{X1}═ in which R^{X1} is a hydrogen atom, and it is more preferable that all of W¹, W², W⁴, and W⁶ to W¹ are —CR^{X1}═ in which R^{X1} is a hydrogen atom.

In Formula (1-7), it is preferable that at least five of W¹, W³, W⁴, and W⁶ to W¹ are —CR^{X1}═ in which R^{X1} is a hydrogen atom, and it is more preferable that all of W¹, W³, W⁴, and W⁶ to W⁸ are —CR^{X1}═ in which R^{X1} is a hydrogen atom.

In Formula (1-1) to Formula (1-7), Rs represents an aliphatic hydrocarbon group having 1 to 20 carbon atoms (preferably 1 to 10 carbon atoms), which may have a substituent, an aromatic ring group which may have a substituent, or an alkoxy group having 1 to 20 carbon atoms, which may have a substituent.

Examples of the aliphatic hydrocarbon group include a linear aliphatic hydrocarbon group, a branched aliphatic hydrocarbon group, and a cyclic aliphatic hydrocarbon group.

The number of carbon atoms of the linear aliphatic hydrocarbon group is preferably 1 to 12, more preferably 1 to 5, and still more preferably 1 to 3.

Examples of the linear aliphatic hydrocarbon group include a linear alkyl group such as a methyl group, an ethyl group, and an n-propyl group.

The number of carbon atoms of the branched aliphatic hydrocarbon group is preferably 3 to 10 and more preferably 3 to 5.

Examples of the branched aliphatic hydrocarbon group include a branched alkyl group such as an isopropyl group, a sec-butyl group, an iso-butyl group, a tert-butyl group, and a neopentyl group.

The number of carbon atoms of the cyclic aliphatic hydrocarbon group is preferably 3 to 10 and more preferably 3 to 8.

Examples of the cyclic aliphatic hydrocarbon group include a cyclic alkyl group (cycloalkyl group) such as a cyclopropyl group, a cyclobutyl group, a cyclopentyl group, a cyclohexyl group, and a cycloheptyl group.

Examples of the substituent which may be included in the linear aliphatic hydrocarbon group, the branched aliphatic hydrocarbon group, and the cyclic aliphatic hydrocarbon group include the substituents exemplified by the substituent W, and a halogen atom is preferable.

The definition of the aromatic ring constituting the above-described aromatic ring group is as described above, and examples of the aromatic ring group include an aryl group and a heteroaryl group. The number of ring members in the aromatic ring constituting the above-described aromatic ring group is preferably 4 to 20, more preferably 4 to 10, and still more preferably 5 or 6.

The number of carbon atoms of the above-described aromatic ring group is not particularly limited, and is preferably 4 to 20 and more preferably 4 to 10.

The above-described aryl group is preferably a phenyl group which may have a substituent.

The above-described heteroaryl group is preferably a pyridine ring group which may have a substituent.

In a case where the aromatic ring group has a substituent, the number of substituents is not particularly limited, and the aromatic ring group may have one substituent or a plurality of substituents.

Examples of the substituent which may be included in the aromatic ring group include the substituents exemplified by the substituent W, and an alkyl group or a halogen atom is preferable.

The number of carbon atoms of the above-described alkoxy group is preferably 1 to 20, more preferably 1 to 10, still more preferably 1 to 6, and particularly preferably 1 to 3.

Examples of the substituent which may be included in the alkoxy group include the substituents exemplified by the substituent W, and a halogen atom is preferable.

Among these, from the viewpoint of being more excellent in the effect of the present invention, Rs is preferably a linear aliphatic hydrocarbon group having 1 to 3 carbon atoms, which may have a halogen atom, a branched aliphatic hydrocarbon group having 3 to 5 carbon atoms, which may have a halogen atom, a cyclic aliphatic hydrocarbon group having 3 to 8 carbon atoms, which may have a halogen atom, an aromatic ring group having 4 to 10 carbon atoms, which may have a substituent, or an alkoxy group having 1 to 3 carbon atoms.

A molecular weight of the above-described specific compound is preferably 400 to 1,200, more preferably 400 to 1,000, and still more preferably 400 to 800.

In a case where the molecular weight is in the above-described range, it is presumed that the sublimation temperature of the specific compound becomes low, and the quantum efficiency is excellent also in a case where a photoelectric conversion film is formed at a high speed.

In the specific compound, an ionization potential in a single film is preferably −6.0 to −5.0 eV from the viewpoints of stability in a case of using the compound as the p-type organic semiconductor and matching of energy levels between the compound and the n-type organic semiconductor.

The maximal absorption wavelength of the specific compound is preferably in a wavelength range of 400 to 600 nm, and more preferably in a wavelength range of 400 to 500 nm.

The maximal absorption wavelength is a value measured in a solution state (solvent: chloroform) by an absorption spectrum of the specific compound being adjusted to a concentration having an absorbance of about 0.5 to 1.0. Provided that in a case where the specific compound is not soluble in chloroform, a value measured by using the specific compound in which the specific compound is vapor-deposited and formed into a film state is defined as a maximal absorption wavelength of the specific compound.

The specific compound is particularly useful as a material of the photoelectric conversion film used for the imaging element, the optical sensor, or a photoelectric cell. The specific compound often functions as a coloring agent in the photoelectric conversion film. The specific compound can also be used as a coloring material, a liquid crystal material, an organic semiconductor material, a charge transport material, a pharmaceutical material, and a fluorescent diagnostic material.

Specific examples of the specific compound are shown below, but the present invention is not limited thereto.

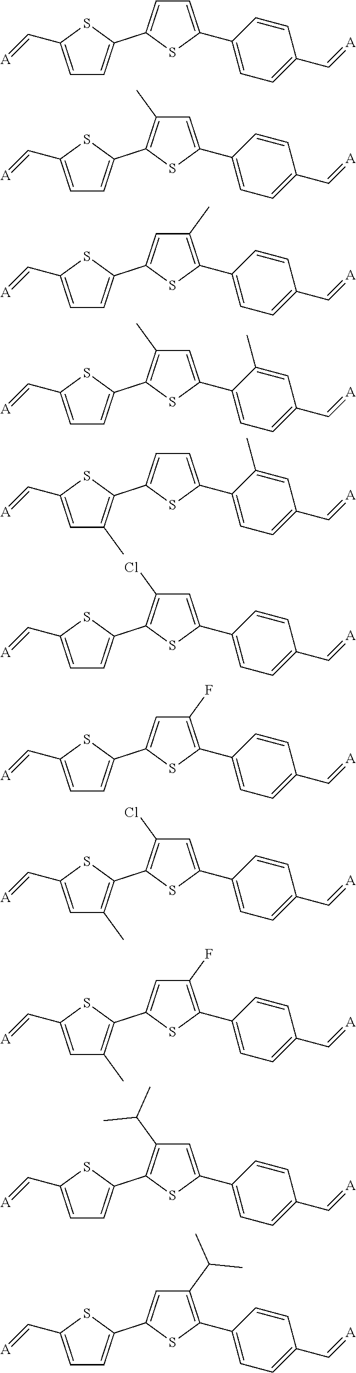

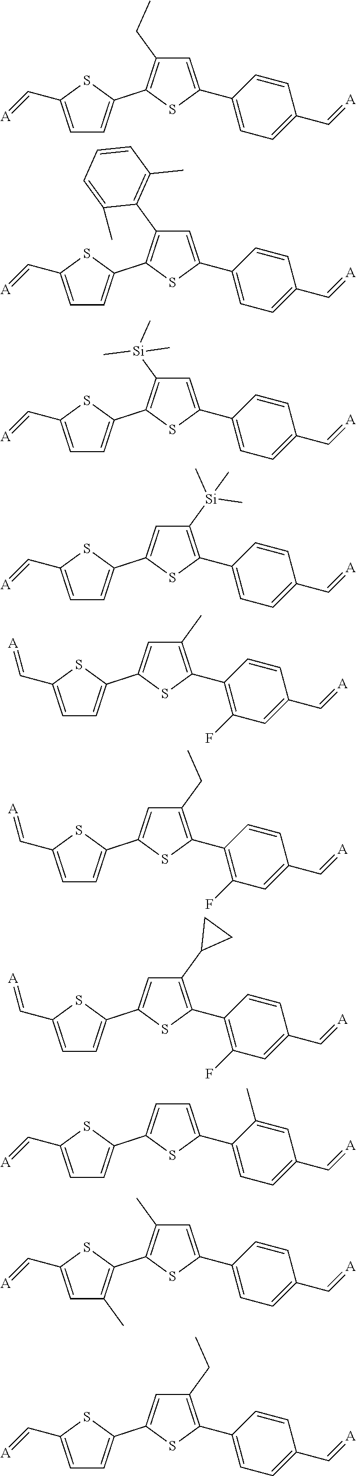

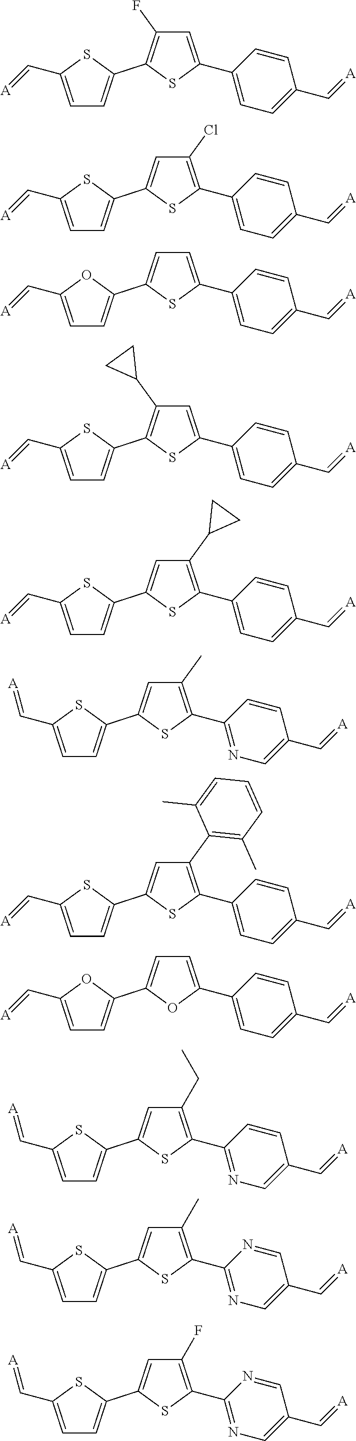

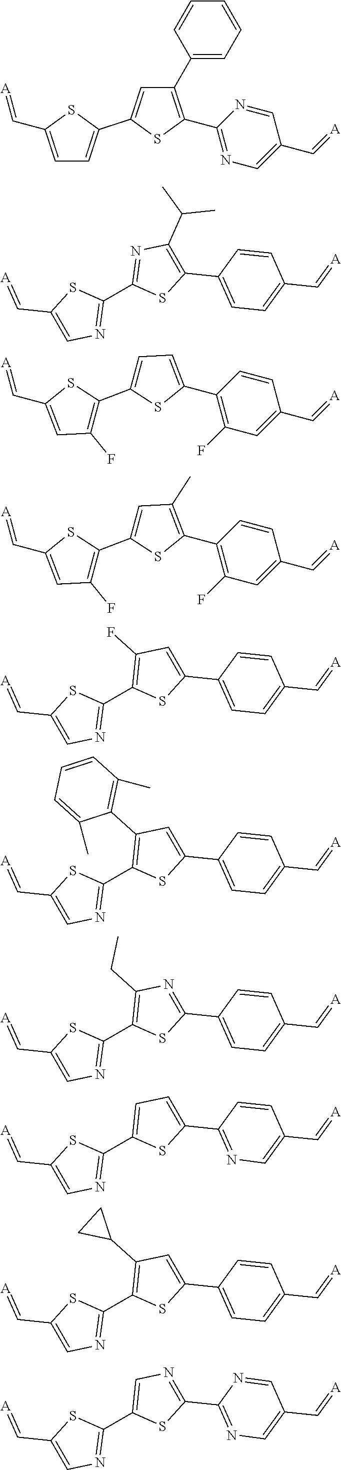

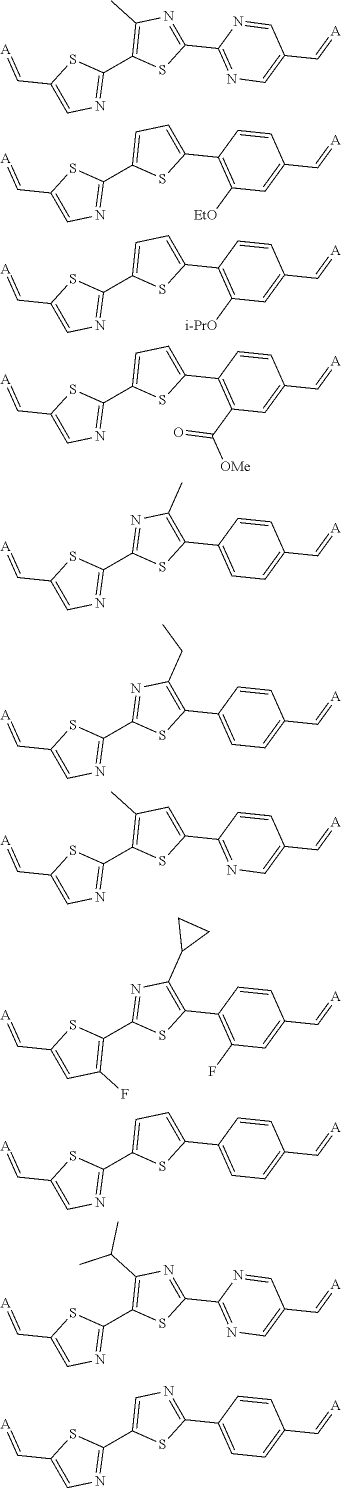

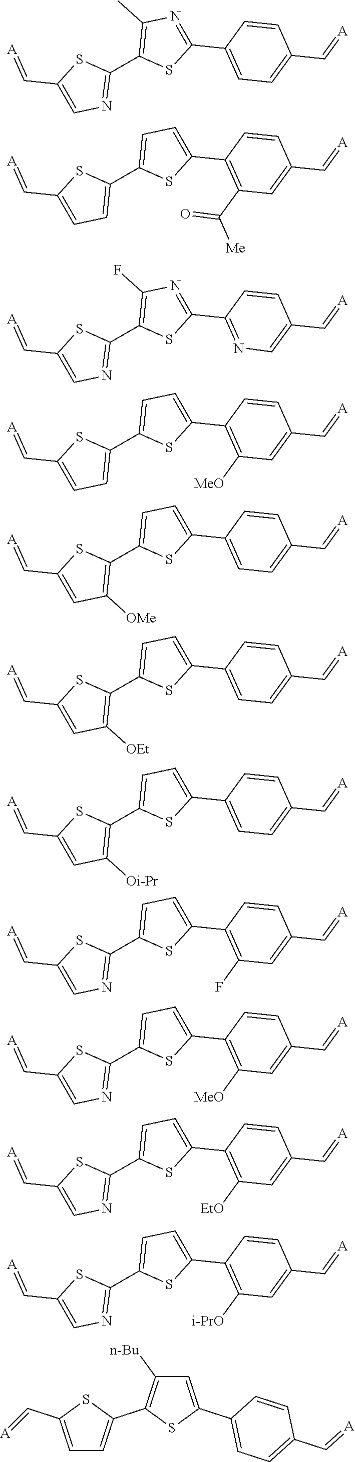

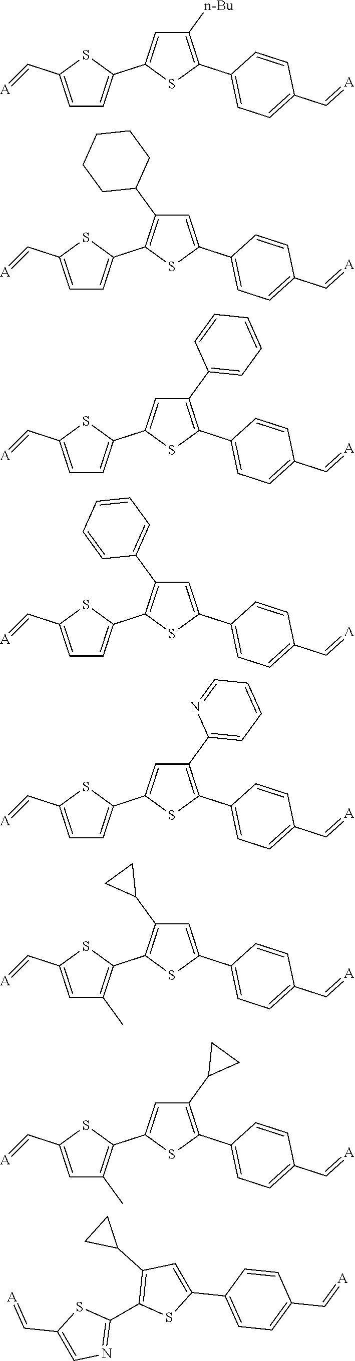

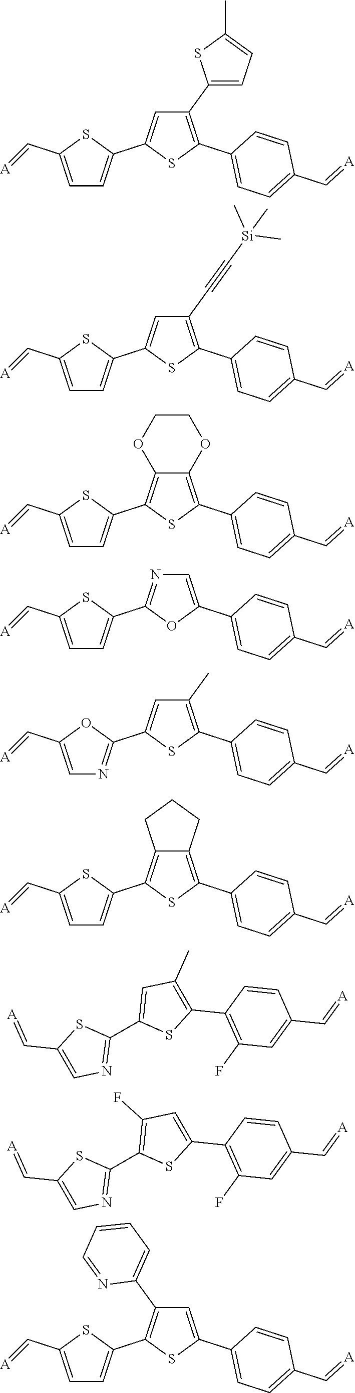

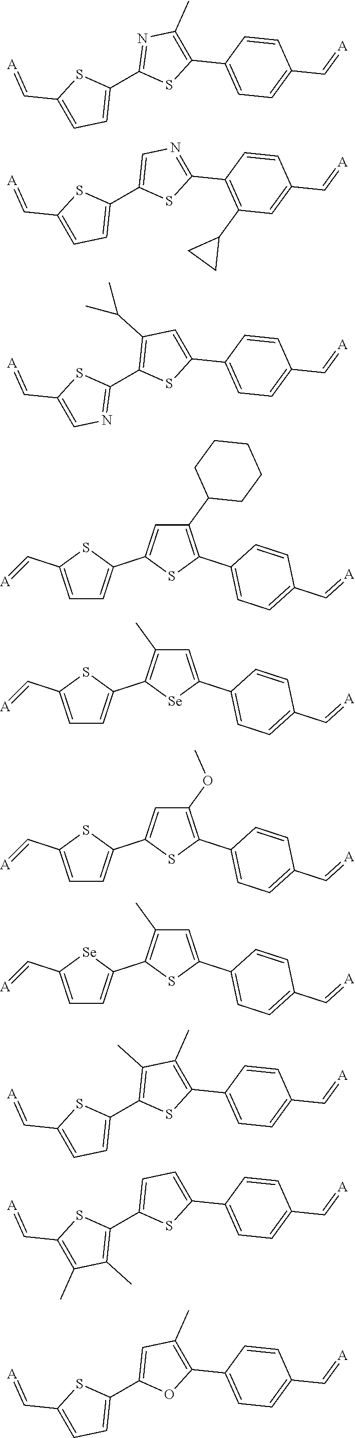

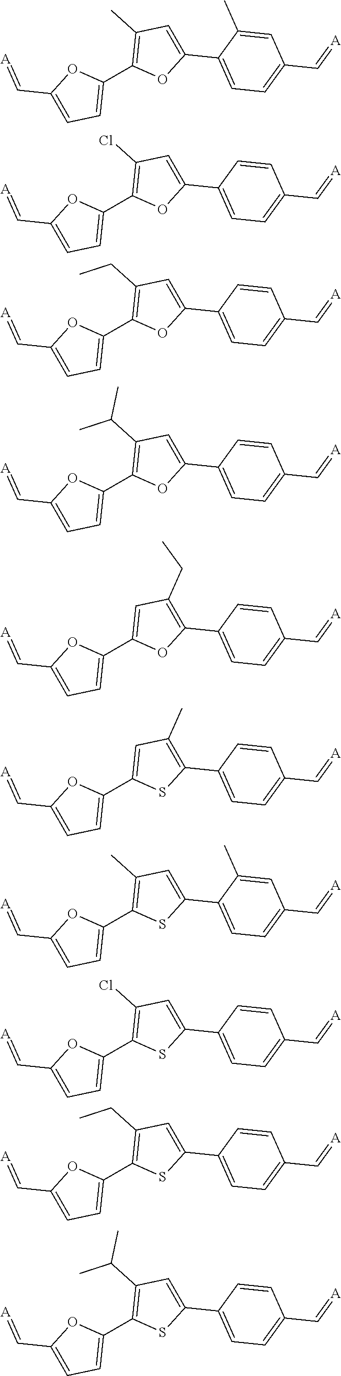

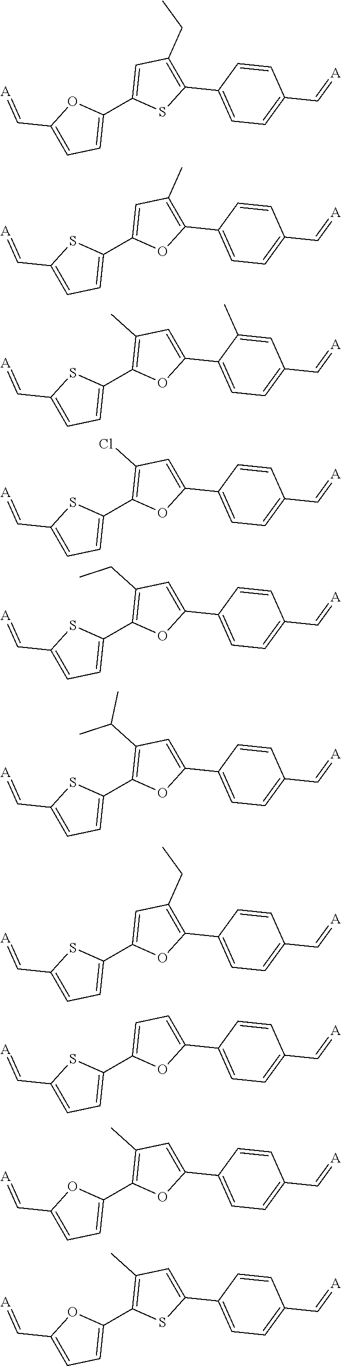

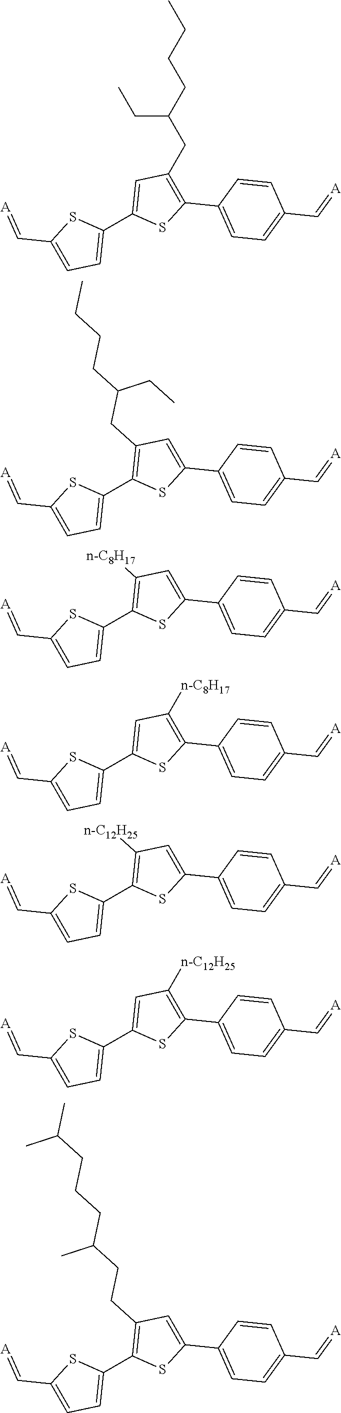

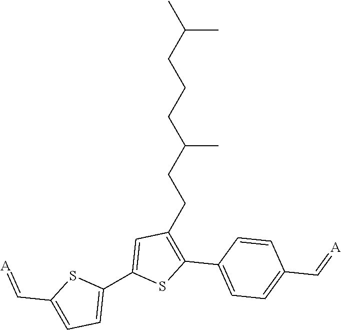

A in the specific compound exemplified above is represented by any of the following groups. * represents a bonding position.

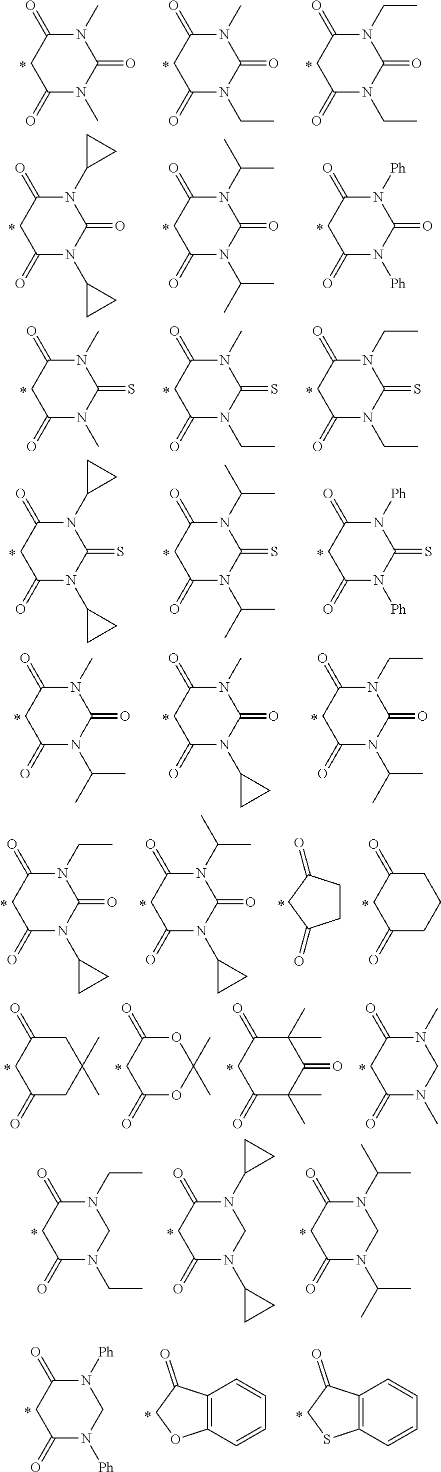

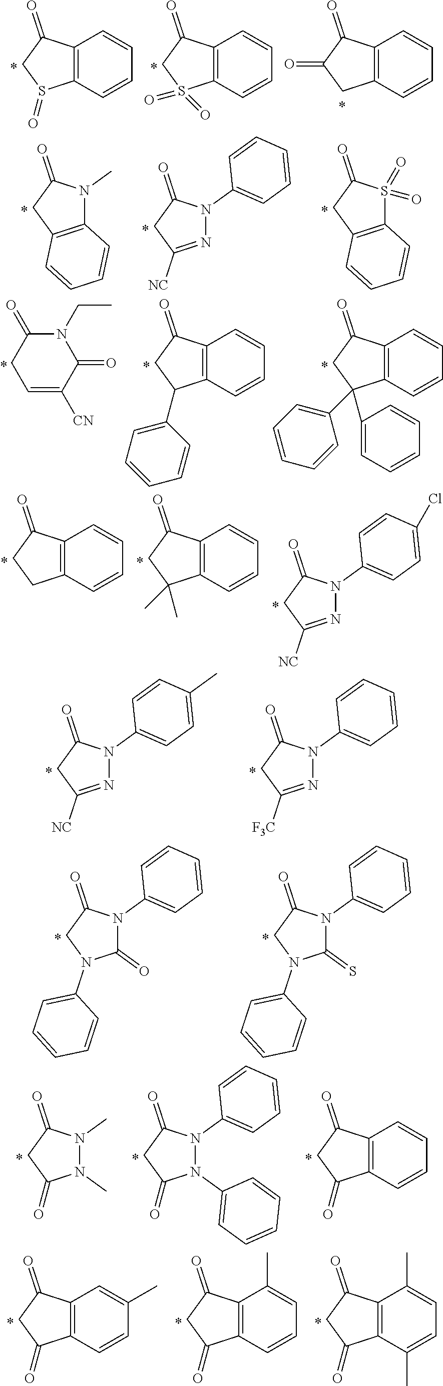

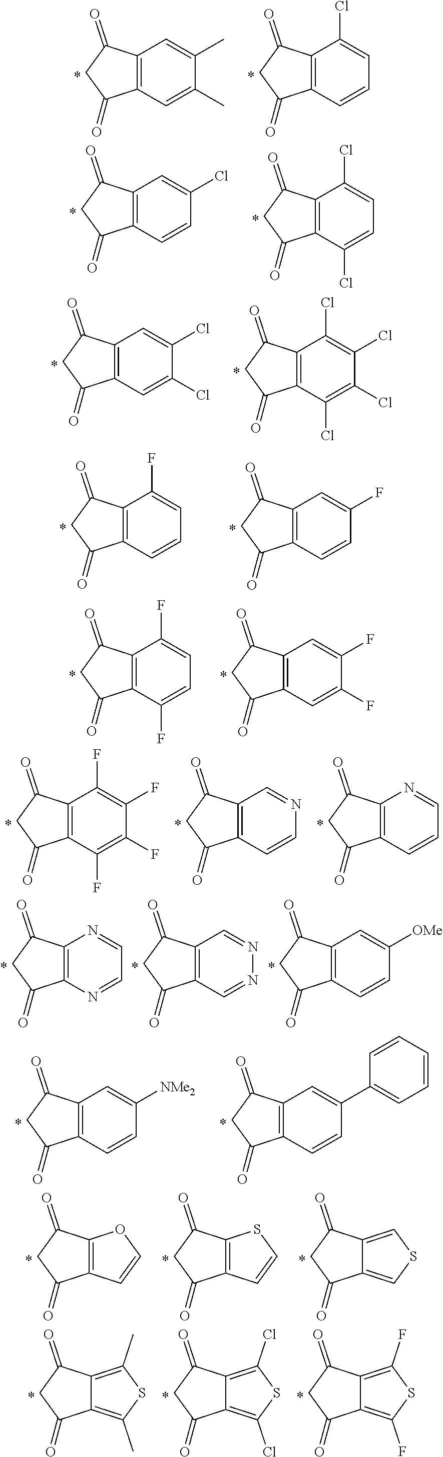

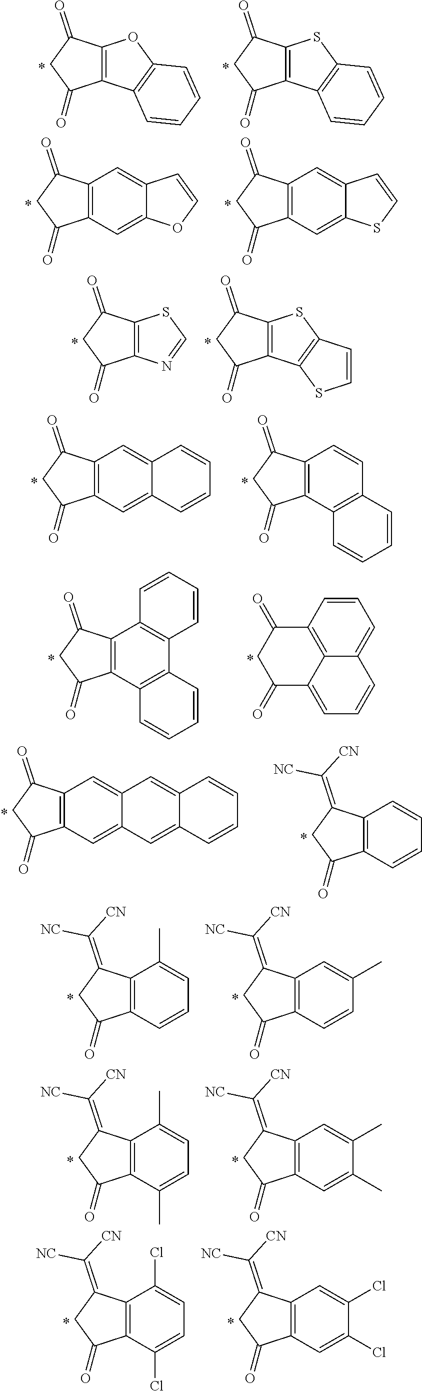

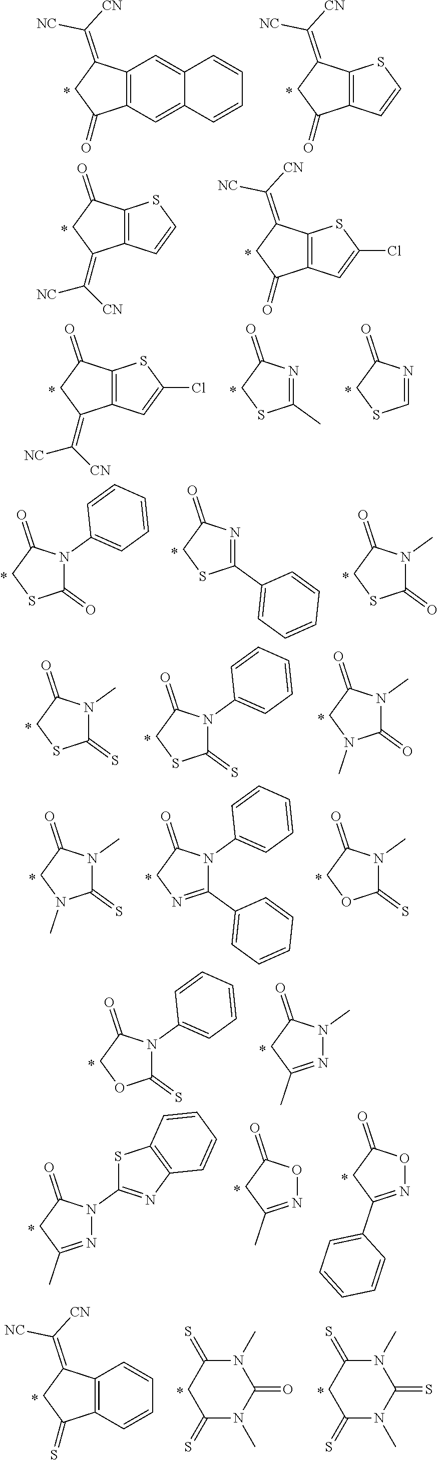

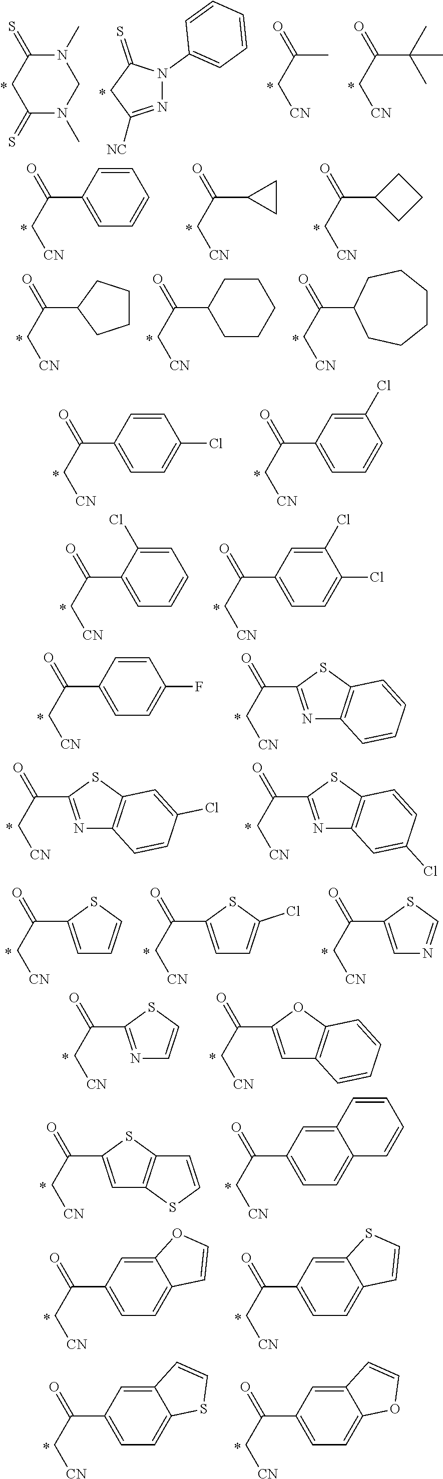

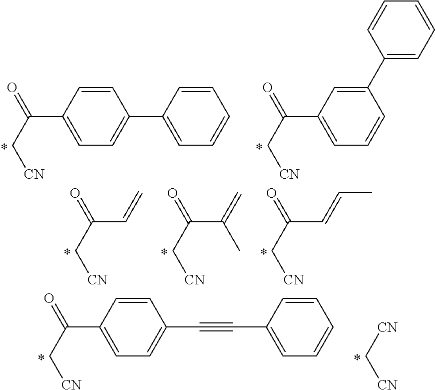

As shown in the following examples, A in each specific compound may be the same or different from each other.

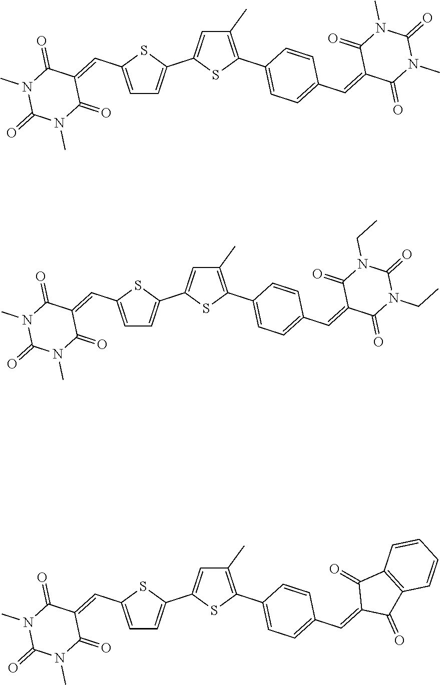

The specific compound may be purified as necessary.

Examples of a purification method of the specific compound include sublimation purification, purification using silica gel column chromatography, purification using gel permeation chromatography, reslurry washing, repurification by reprecipitation, purification using an adsorbent such as activated carbon, and recrystallization purification.

A content of the specific compound in the photoelectric conversion film (=film thickness of specific compound in terms of single layer/film thickness of photoelectric conversion film×100) is not particularly limited, and is preferably 15% to 75% by volume, more preferably 20% to 60% by volume, and still more preferably 20% to 50% by volume.

The specific compound may be used alone or in combination of two or more types thereof. In a case where two or more types thereof are used, the total amount thereof is preferably within the above-described range.

<n-Type Organic Semiconductor>

The photoelectric conversion film preferably contains the n-type organic semiconductor in addition to the specific compound.

The n-type organic semiconductor is a compound different from the specific compound.

The n-type organic semiconductor is an acceptor-property organic semiconductor material (a compound), and refers to an organic compound having a property of easily accepting an electron. That is, the n-type organic semiconductor refers to an organic compound having a large electron affinity of two organic compounds used in contact with each other. That is, any organic compound having an electron accepting property can be used as the acceptor type organic semiconductor.

Examples of the n-type organic semiconductor include fullerenes selected from the group consisting of a fullerene and derivatives thereof, fused aromatic carbocyclic compounds (for example, a naphthalene derivative, an anthracene derivative, a phenanthrene derivative, a tetracene derivative, a pyrene derivative, a perylene derivative, and a fluoranthene derivative); a heterocyclic compound having a 5- to 7-membered ring having at least one selected from the group consisting of a nitrogen atom, an oxygen atom, and a sulfur atom (for example, pyridine, pyrazine, pyrimidine, pyridazine, triazine, quinoline, quinoxaline, quinazoline, phthalazine, cinnoline, isoquinoline, pteridine, acridine, phenazine, phenanthroline, tetrazole, pyrazole, imidazole, and thiazole); polyarylene compounds; fluorene compounds; cyclopentadiene compounds; silyl compounds; 1,4,5,8-naphthalenetetracarboxylic acid anhydride; 1,4,5,8-naphthalenetetracarboxylic acid diimide derivatives; anthraquinodimethane derivatives; diphenylquinone derivatives; bathocuproine, bathophenanthroline, and derivatives thereof, triazole compounds; a distyrylarylene derivative; a metal complex having a nitrogen-containing heterocyclic compound as a ligand; a silole compound; 3,4,9,10-perylenetetracarboxylic acid dianhydride; 3,4,9,10-perylenetetracarboxylic diimide derivative; and compounds disclosed in paragraphs [0056] to [0057] of JP2006-100767A.

The n-type organic semiconductor (compound) is preferably fullerenes selected from the group consisting of a fullerene and derivatives thereof.

Examples of the fullerenes include a fullerene C₆₀, a fullerene C₇₀, a fullerene C₇₆, a fullerene C₇₈, a fullerene C₈₀, a fullerene C₈₂, a fullerene C₈₄, a fullerene C₉₀, a fullerene C₉₆, a fullerene C₂₄₀, a fullerene C₅₄₀, and a mixed fullerene.

Examples of the fullerene derivatives include compounds in which a substituent is added to the above fullerenes. The substituent is preferably an alkyl group, an aryl group, or a heterocyclic group. As the fullerene derivative, the compounds described in JP2007-123707A are preferable.

The n-type organic semiconductor may be an organic coloring agent.

Examples of the organic coloring agent include a cyanine coloring agent, a styryl coloring agent, a hemicyanine coloring agent, a merocyanine coloring agent (including zeromethine merocyanine (simple merocyanine)), a rhodacyanine coloring agent, an allopolar coloring agent, an oxonol coloring agent, a hemioxonol coloring agent, a squarylium coloring agent, a croconium coloring agent, an azamethine coloring agent, a coumarin coloring agent, an arylidene coloring agent, an anthraquinone coloring agent, a triphenylmethane coloring agent, an azo coloring agent, an azomethine coloring agent, a metallocene coloring agent, a fluorenone coloring agent, a flugide coloring agent, a perylene coloring agent, a phenazine coloring agent, a phenothiazine coloring agent, a quinone coloring agent, a diphenylmethane coloring agent, a polyene coloring agent, an acridine coloring agent, an acridinone coloring agent, a diphenylamine coloring agent, a quinophthalone coloring agent, a phenoxazine coloring agent, a phthaloperylene coloring agent, a dioxane coloring agent, a porphyrin coloring agent, a chlorophyll coloring agent, a phthalocyanine coloring agent, a subphthalocyanine coloring agent, and a metal complex coloring agent.

The molecular weight of the n-type organic semiconductor is preferably 200 to 1,200, and more preferably 200 to 900.

A maximal absorption wavelength of the n-type organic semiconductor is preferably in a wavelength of 400 nm or less or in a wavelength of more than 400 nm and 600 nm or less.

It is preferable that the photoelectric conversion film has a bulk hetero structure formed in a state in which the specific compound and the n-type organic semiconductor are mixed. The bulk hetero structure refers to a layer in which the specific compound and the n-type organic semiconductor are mixed and dispersed in the photoelectric conversion film. The photoelectric conversion film having the bulk hetero structure can be formed by either a wet method or a dry method. The bulk hetero structure is described in detail in, for example, paragraphs [0013] and [0014] of JP2005-303266A.

The difference in electron affinity between the specific compound and the n-type organic semiconductor is preferably 0.1 eV or more.

The n-type organic semiconductor may be used alone, or two or more types thereof may be used in combination.

In a case where the photoelectric conversion film contains the n-type organic semiconductor, a content of the n-type organic semiconductor in the photoelectric conversion film (film thickness of n-type organic semiconductor in terms of single layer/film thickness of photoelectric conversion film×100) is preferably 15% to 75% by volume, more preferably 20% to 60% by volume, and still more preferably 20% to 50% by volume.

In a case where the n-type organic semiconductor material includes fullerenes, a content of the fullerenes to a total content of the n-type organic semiconductor material (film thickness of fullerenes in terms of single layer/total film thickness of n-type organic semiconductor materials in terms of single layer×100) is preferably 50% to 100% by volume, and more preferably 80% to 100% by volume. The fullerenes may be used alone, or two or more types thereof may be used in combination.

From the viewpoint of response speed of the photoelectric conversion element, the content of the specific compound to the total content of the specific compound and the n-type organic semiconductor (film thickness in terms of single layer of specific compound/(film thickness in terms of single layer of specific compound+film thickness in terms of single layer of n-type organic semiconductor)×100) is preferably 20% to 80% by volume, and more preferably 40% to 80% by volume.

In a case where the photoelectric conversion film contains an n-type organic semiconductor and a p-type organic semiconductor, the content of the specific compound (=film thickness in terms of single layer of specific compound/(film thickness in terms of single layer of specific compound+film thickness in terms of single layer of n-type organic semiconductor+film thickness in terms of single layer of p-type organic semiconductor)×100) is preferably 15% to 75% by volume, and more preferably 30% to 75% by volume. It is preferable that the photoelectric conversion film is substantially formed of the specific compound, the n-type organic semiconductor, and the p-type organic semiconductor included as desired. The term “substantially” indicates that the total content of the specific compound, the n-type organic semiconductor, and the p-type organic semiconductor is 90% to 100% by volume, preferably 95% to 100% by volume, and more preferably 99% to 100% by volume, with respect to the total mass of the photoelectric conversion film.

<p-Type Organic Semiconductor>

The photoelectric conversion film preferably contains the p-type organic semiconductor in addition to the specific compound.

The p-type organic semiconductor is a compound different from the specific compound. The p-type organic semiconductor is a donor organic semiconductor material (a compound), and refers to an organic compound having a property of easily donating an electron. That is, the p-type organic semiconductor means an organic compound having a smaller ionization potential in a case where two organic compounds are used in contact with each other.

The p-type organic semiconductor may be used alone, or two or more types thereof may be used in combination.

Examples of the p-type organic semiconductor include triarylamine compounds (for example, N,N′-bis(3-methylphenyl)-(1,1′-biphenyl)-4,4′-diamine (TPD), 4,4′-bis[N-(naphthyl)-N-Phenyl-amino]biphenyl (α-NPD), compounds disclosed in paragraphs [0128] to [0148] of JP2011-228614A, compounds disclosed in paragraphs [0052] to [0063] of JP2011-176259A, compounds disclosed in paragraphs [0119] to [0158] of JP2011-225544A, compounds disclosed in paragraphs [0044] to [0051] of JP2015-153910A, and compounds disclosed in paragraphs [0086] to [0090] of JP2012-094660A), pyrazoline compounds, styrylamine compounds, hydrazone compounds, polysilane compounds, thiophene compounds (for example, a thienothiophene derivative, a dibenzothiophene derivative, a benzodithiophene derivative, a dithienothiophene derivative, a [1]benzothieno[3,2-b]thiophene (BTBT) derivative, a thieno [3,2-f: 4,5-f]bis [1]benzothiophene (TBBT) derivative, compounds disclosed in paragraphs [0031] to [0036] of JP2018-014474A, compounds disclosed in paragraphs [0043] to [0045] of WO2016/194630A, compounds disclosed in paragraphs [0025] to [0037], and [0099] to [0109] of WO2017/159684A, compounds disclosed in paragraphs [0029] to [0034] of JP2017-076766A, compounds disclosed in paragraphs [0015] to [0025] of WO2018/207722A, compounds disclosed in paragraphs [0045] to [0053] of JP2019-054228A, compounds disclosed in paragraphs [0045] to [0055] of WO2019/058995A, compounds disclosed in paragraphs [0063] to [0089] of WO2019/081416A, compounds disclosed in paragraphs [0033] to [0036] of JP2019-80052A, compounds disclosed in paragraphs [0044] to [0054] of WO2019/054125A, compounds disclosed in paragraphs [0041] to [0046] of WO2019/093188A and the like), compounds in paragraphs [0034] to [0037] of JP2019-050398A, compounds in paragraphs [0033] to [0036] of JP2018-206878A, compounds in paragraph [0038] of JP2018-190755A, compounds in paragraphs [0019] to [0021] of JP2018-026559A, compounds in paragraphs [0031] to [0056] of JP2018-170487A, compounds in paragraphs [0036] to [0041] of JP2018-078270A, compounds in paragraphs [0055] to [0082] of JP2018-166200A, compounds in paragraphs [0041] to [0050] of JP2018-113425A, compounds in paragraphs [0044] to [0048] of JP2018-085430A, compounds in paragraphs [0041] to [0045] of JP2018-056546A, compounds in paragraphs [0042] to [0049] of JP2018-046267A, compounds in paragraphs [0031] to [0036] of JP2018-014474A, compounds disclosed in paragraphs [0036] to [0046] of WO2018/016465A, compounds disclosed in paragraphs [0045] to [0048] of JP2020-010024A, and the like), a cyanine compound, an oxonol compound, a polyamine compound, an indole compound, a pyrrole compound, a pyrazole compound, a polyarylene compound, a fused aromatic carbocyclic compound (for example, a naphthalene derivative, an anthracene derivative, a phenanthrene derivative, a tetracene derivative, a pentacene derivative, a pyrene derivative, a perylene derivative, a fluoranthene derivative, and the like), a porphyrin compound, a phthalocyanine compound, a triazole compound, an oxadiazole compound, an imidazole compound, a polyarylalkane compound, a pyrazolone compound, an amino-substituted chalcone compound, an oxazole compound, a fluorenone compound, a silazane compound, and a metal complex having nitrogen-containing heterocyclic compounds as ligands.

As the p-type organic semiconductor, in addition to the above-described compounds, compounds described in JP2021-163968A, JP2022-027575A, JP2022-123944A, JP2022-122839A, JP2022-120323A, JP2022-120273A, JP2022-115832A, JP2022-108268A, JP2022-100258A, JP2022-181226A, and JP2023-005703A can also be used, the compounds of which are incorporated in the present specification.

Examples of the p-type organic semiconductor also include compounds having an ionization potential smaller than that of the n-type organic semiconductor, and in a case where this condition is satisfied, the organic coloring agents exemplified as the n-type organic semiconductor can be used.

The compounds that can be used as the p-type organic semiconductor compound are exemplified below.

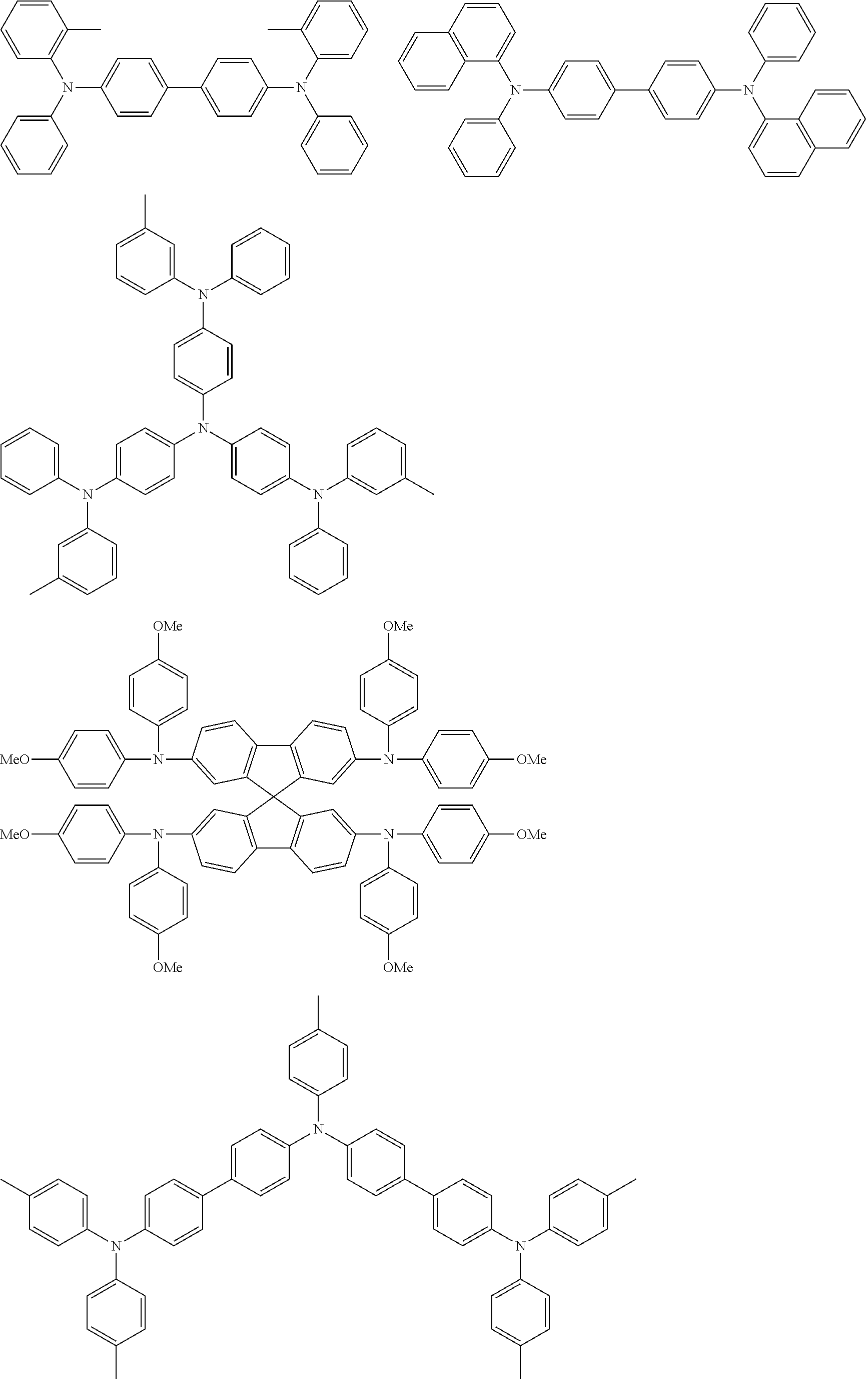

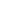

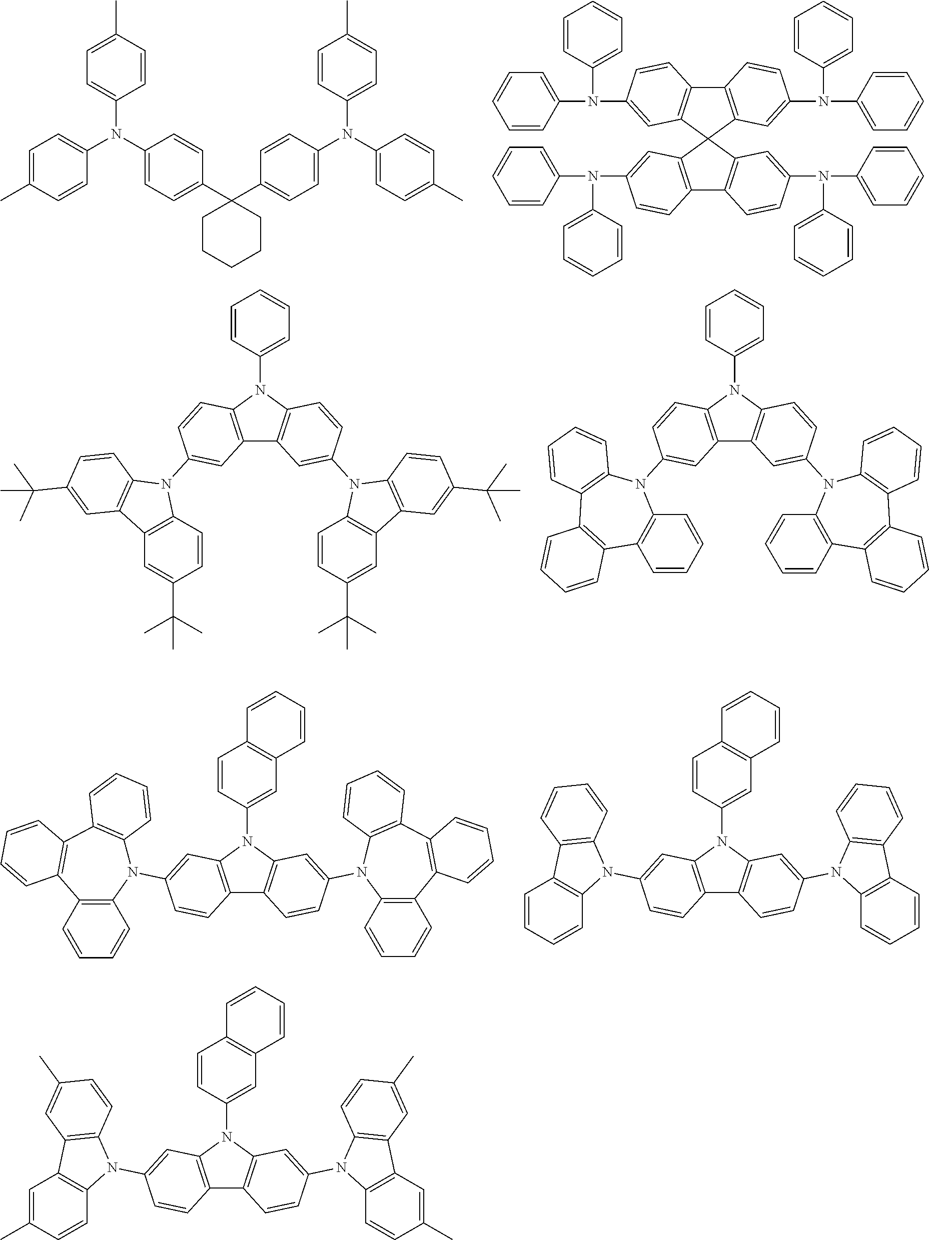

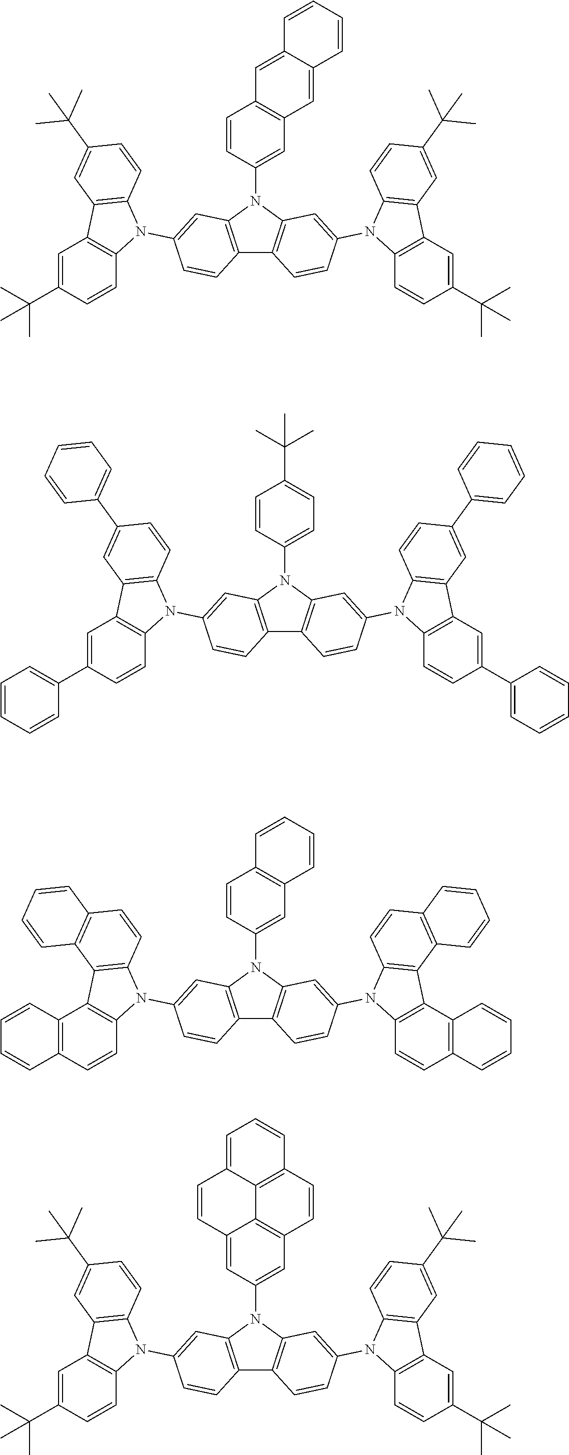

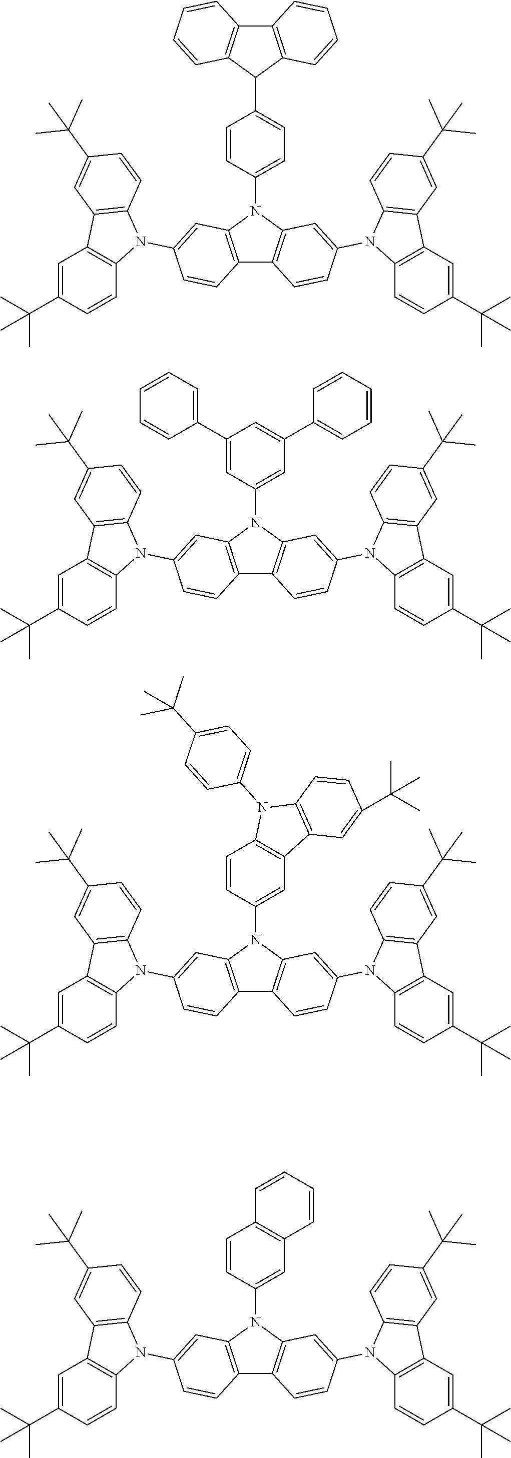

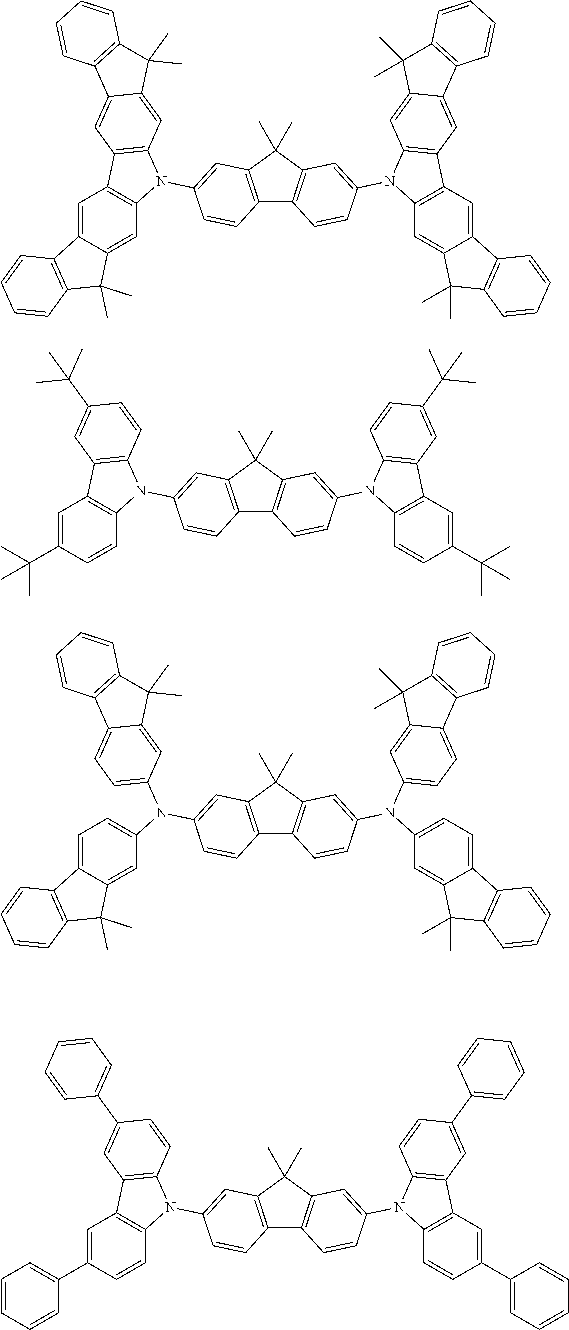

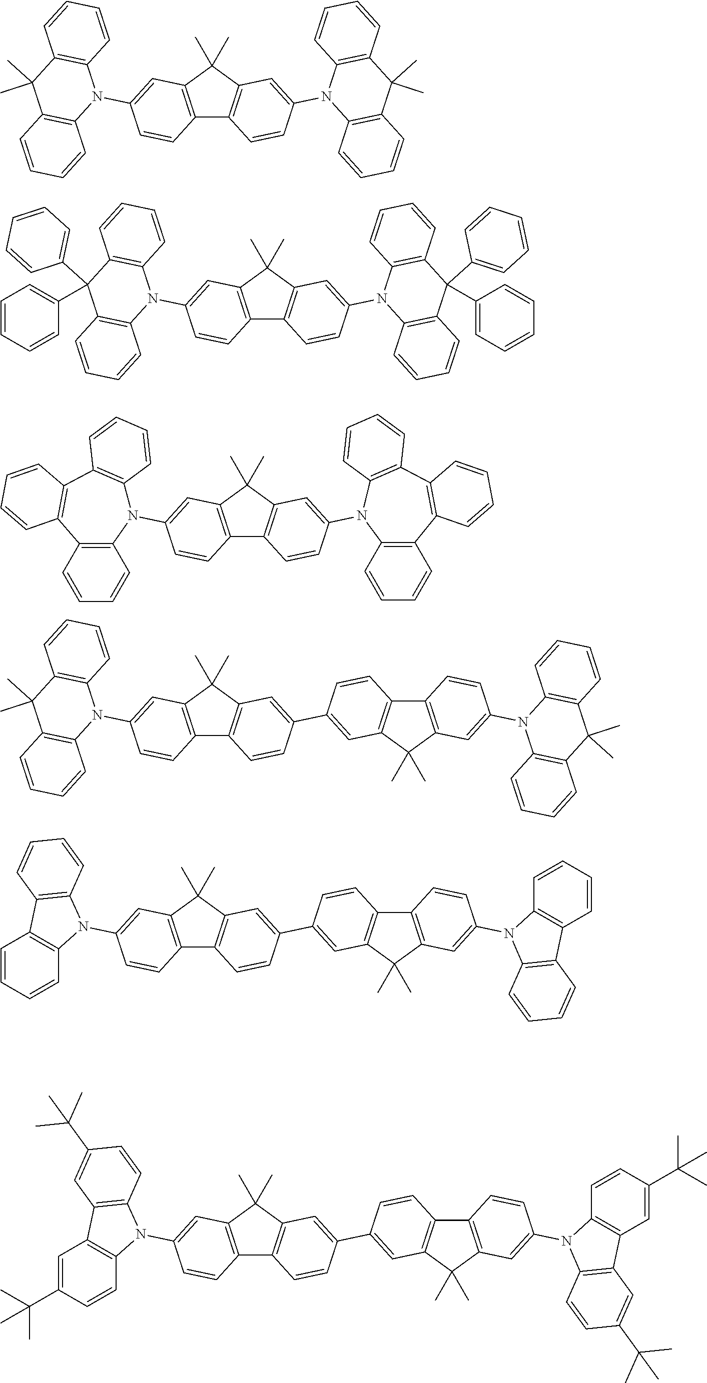

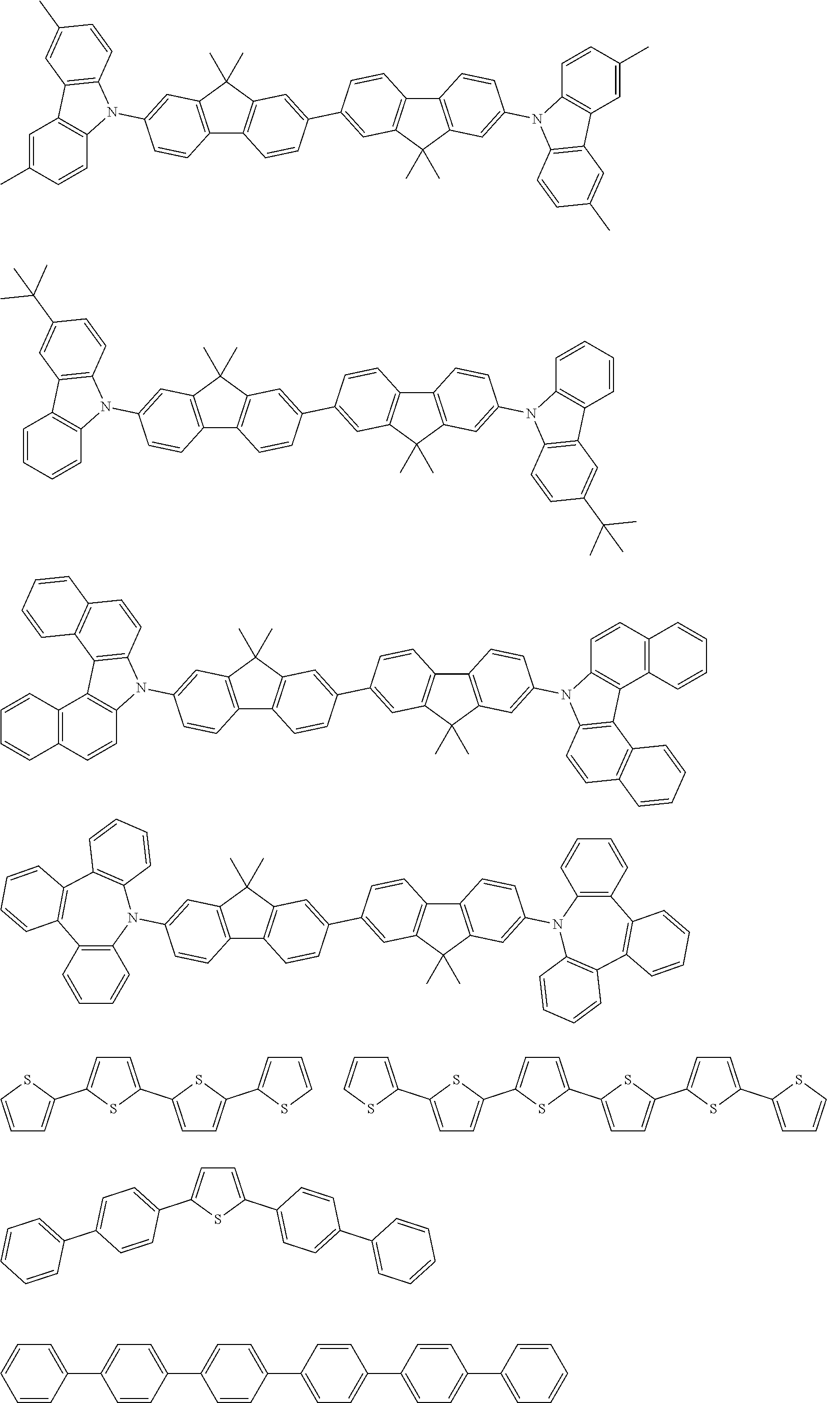

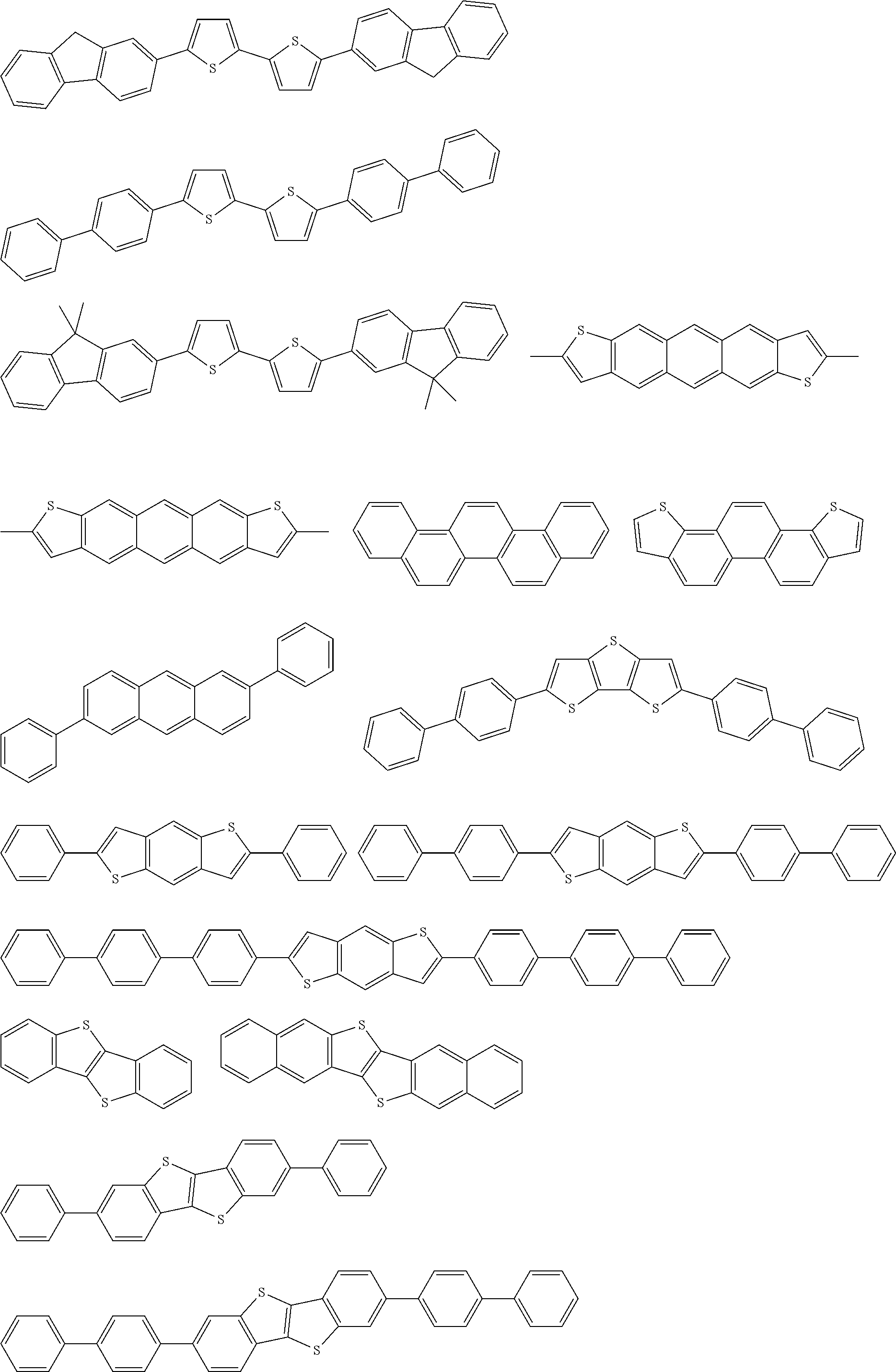

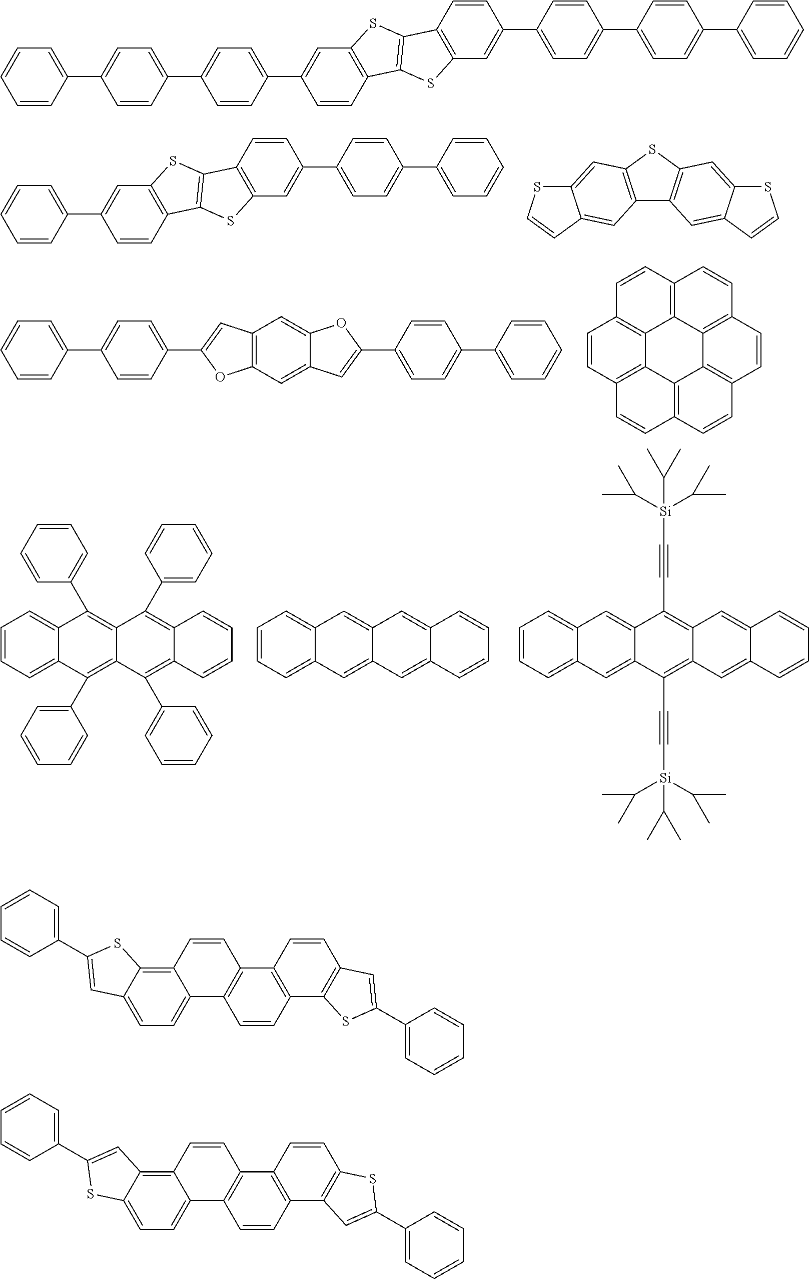

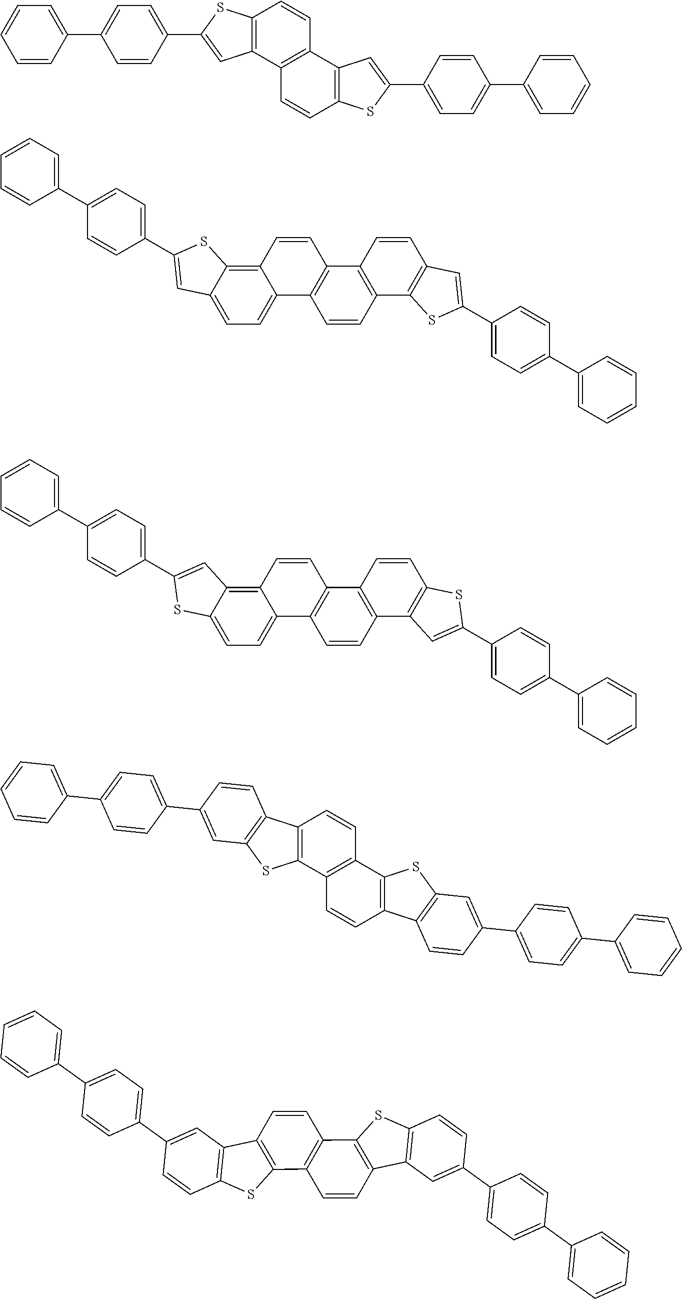 [SMILES: c1ccc(-c2ccc(-c3cc4c(ccc5c6cc(-c7ccc(-c8ccccc8)cc7)sc6ccc45)s3)cc2)cc1 ; c1ccc(-c2ccc(-c3cc4ccc5c6ccc7c(ccc8cc(-c9ccc(-c%10ccccc%10)cc9)sc87)c6ccc5c4s3)cc2)cc1 ; c1ccc(-c2ccc(-c3cc4c(ccc5c4ccc4c6ccc7sc(-c8ccc(-c9ccccc9)cc8)cc7c6ccc54)s3)cc2)cc1 ; c1ccc(-c2ccc(-c3ccc4c(c3)sc3c4ccc4c3ccc3c5ccc(-c6ccc(-c7ccccc7)cc6)cc5sc34)cc2)cc1 ; c1ccc(-c2ccc(-c3ccc4sc5c(ccc6c5ccc5c7cc(-c8ccc(-c9ccccc9)cc8)ccc7sc56)c4c3)cc2)cc1]

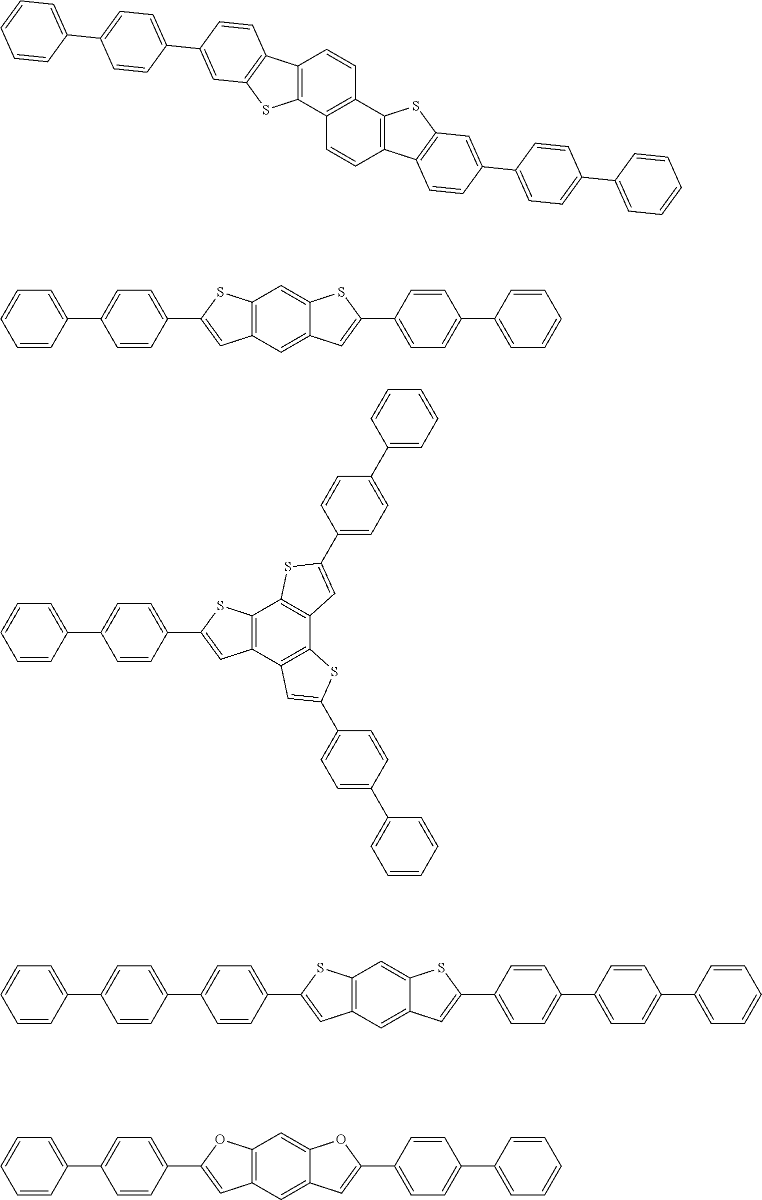

The difference in the ionization potential between the specific compound and the p-type organic semiconductor is preferably 0.1 eV or more.

The p-type organic semiconductor material may be used alone, or two or more types thereof may be used in combination.

In a case where the photoelectric conversion film contains the p-type organic semiconductor, a content of the p-type organic semiconductor in the photoelectric conversion film (film thickness of p-type organic semiconductor in terms of single layer/film thickness of photoelectric conversion film×100) is preferably 15% to 75% by volume, more preferably 20% to 60% by volume, and still more preferably 25% to 50% by volume.

The photoelectric conversion film containing the specific compound is a non-light emitting film, and has a feature different from organic light emitting diodes (OLEDs). The non-light emitting film means a film having a light emission quantum efficiency of 1% or less, and the light emission quantum efficiency is preferably 0.5% or less, and more preferably 0.1% or less. The lower limit thereof is often 0% or more.

### <Coloring Agent>

The photoelectric conversion film preferably contains a coloring agent in addition to the above-described specific compound.

The coloring agent is a compound different from the specific compound.

As the coloring agent, an organic coloring agent is preferable.

Examples of the organic coloring agent include a cyanine coloring agent, a styryl coloring agent, a hemicyanine coloring agent, a merocyanine coloring agent (including zeromethine merocyanine (simple merocyanine)), a rhodacyanine coloring agent, an allopolar coloring agent, an oxonol coloring agent, a hemioxonol coloring agent, a squarylium coloring agent, a croconium coloring agent, an azamethine coloring agent, a coumarin coloring agent, an arylidene coloring agent, an anthraquinone coloring agent, a triphenylmethane coloring agent, an azo coloring agent, an azomethine coloring agent, a metallocene coloring agent, a fluorenone coloring agent, a flugide coloring agent, a perylene coloring agent, a phenazine coloring agent, a phenothiazine coloring agent, a quinone coloring agent, a diphenylmethane coloring agent, a polyene coloring agent, an acridine coloring agent, an acridinone coloring agent, a diphenylamine coloring agent, a quinophthalone coloring agent, a phenoxazine coloring agent, a phthaloperylene coloring agent, a dioxane coloring agent, a porphyrin coloring agent, a chlorophyll coloring agent, a phthalocyanine coloring agent, a subphthalocyanine coloring agent, a metal complex coloring agent, an imidazoquinoxaline coloring agent described in WO2020/013246A, WO2022/168856A, JP2023-10305A, and JP2023-10299A, acceptor-donor-acceptor type coloring agent in which two acidic nuclei are bonded to a donor, and donor-acceptor-donor type coloring agent in which two donors are bonded to an acceptor, and the like.

Among these, from the viewpoints of having a maximal absorption wavelength in a preferred range described later, and the like, the organic colorant is preferably a cyanine colorant, an imidazoquinoxaline coloring agent, or an acceptor-donor-acceptor type coloring agent.

The maximal absorption wavelength of the coloring agent is preferably in the visible light region, more preferably in a wavelength range of 400 to 650 nm, and still more preferably in a wavelength range of 450 to 650 nm.

The coloring agent may be used alone, or two or more types thereof may be used in combination.

A content of the coloring agent with respect to the total content of the specific compound and the coloring agent in the photoelectric conversion film (=(film thickness of coloring agent in terms of single layer/(film thickness of specific compound in terms of single layer+film thickness of coloring agent in terms of single layer)×100)) is preferably 15% to 75% by volume, more preferably 20% to 60% by volume, and still more preferably 20% to 50% by volume.

### <Film Formation Method>

Examples of a film formation method of the above-described photoelectric conversion film include a dry film formation method.

Examples of the dry film formation method include a physical vapor deposition method such as a vapor deposition method (particularly, a vacuum vapor deposition method), a sputtering method, an ion plating method, and a molecular beam epitaxy (MBE) method, and a chemical vapor deposition (CVD) method such as plasma polymerization, and the vacuum vapor deposition method is preferable. In a case where the photoelectric conversion film is formed by the vacuum vapor deposition method, manufacturing conditions such as a degree of vacuum and a vapor deposition temperature can be set according to the normal method.

The film thickness of the photoelectric conversion film is preferably 10 to 1,000 nm, more preferably 50 to 800 nm, and still more preferably 50 to 500 nm.

### [Electrode]

The photoelectric conversion element preferably has an electrode.

Electrodes (the upper electrode (the transparent conductive film) 15 and the lower electrode (the conductive film) 11) are formed of conductive materials. Examples of the conductive material include metals, alloys, metal oxides, electrically conductive compounds, and mixtures thereof.

Since light is incident through the upper electrode 15, the upper electrode 15 is preferably transparent to light to be detected. Examples of the materials constituting the upper electrode 15 include conductive metal oxides such as tin oxide (antimony tin oxide (ATO) and fluorine doped tin oxide (FTO)) doped with antimony, fluorine or the like, tin oxide, zinc oxide, indium oxide, indium tin oxide (ITO), and indium zinc oxide (IZO); metal thin films such as gold, silver, chromium, and nickel; mixtures or laminates of these metals and the conductive metal oxides; and organic conductive materials such as polyaniline, polythiophene, and polypyrrole; and nano carbon materials such as carbon nanotubes, graphene, and the like. From the viewpoint of high conductivity and transparency, conductive metal oxides are preferable.

Generally, in a case where the conductive film is made thinner than a certain range, the resistance value rapidly increases in many cases. In the solid-state imaging element in which the photoelectric conversion element according to the present embodiment is incorporated, the sheet resistance may be 100 to 10,000Ω/□, and the degree of freedom of the film thickness range that can be reduced is large.

In addition, as the film thickness of the upper electrode (the transparent conductive film) 15 is thinner, the amount of light that the upper electrode absorbs is smaller, and the light transmittance usually increases. The increase in the light transmittance causes an increase in light absorbance in the photoelectric conversion film and an increase in the photoelectric conversion ability, which is preferable. Considering the suppression of leakage current, an increase in the resistance value of the thin film, and an increase in transmittance accompanied by the thinning, the thickness of the upper electrode 15 is preferably 5 to 100 nm, and more preferably 5 to 20 nm.

There is a case where the lower electrode 11 has transparency or an opposite case where the lower electrode 11 does not have transparency and reflects light, depending on the application. Examples of a material constituting the lower electrode 11 include conductive metal oxides such as tin oxide (ATO and FTO) doped with antimony, fluorine, or the like, tin oxide, zinc oxide, indium oxide, indium tin oxide (ITO), and indium zinc oxide (IZO); metals such as gold, silver, chromium, nickel, titanium, tungsten, and aluminum; conductive compounds (for example, titanium nitride (TiN)) such as oxides or nitrides of these metals; mixtures or laminates of these metals and conductive metal oxides; organic conductive materials such as polyaniline, polythiophene, and polypyrrole; and carbon materials such as carbon nanotubes and graphene.

The method of forming electrodes can be appropriately selected in accordance with the electrode material. Specific examples thereof include a wet method such as a printing method and a coating method; a physical method such as a vacuum vapor deposition method, a sputtering method, and an ion plating method; and a chemical method such as a CVD method and a plasma CVD method.

In a case where the material of the electrode is ITO, examples thereof include an electron beam method, a sputtering method, a resistance heating vapor deposition method, a chemical reaction method (such as a sol-gel method), and a coating method with a dispersion of indium tin oxide.

### [Charge Blocking Film: Electron Blocking Film and Positive Hole Blocking Film]

It is preferable that the photoelectric conversion element includes one or more interlayers between the conductive film and the transparent conductive film, in addition to the photoelectric conversion film.

An example of the interlayer includes a charge blocking film. In a case where the photoelectric conversion element has this film, the characteristics (such as quantum efficiency and response speed) of the photoelectric conversion element to be obtained are more excellent. Examples of the charge blocking film include an electron blocking film and a positive hole blocking film.

### [Electron Blocking Film]

The electron blocking film is a donor organic semiconductor material (a compound), and the p-type organic semiconductor described above can be used.

A polymer material can also be used as the electron blocking film.

Examples of the polymer material include a polymer such as phenylenevinylene, fluorene, carbazole, indole, pyrene, pyrrole, picoline, thiophene, acetylene, and diacetylene, and a derivative thereof.

The electron blocking film may be formed of a plurality of films.

The electron blocking film may be formed of an inorganic material. In general, since an inorganic material has a dielectric constant larger than that of an organic material, in a case where the inorganic material is used in the electron blocking film, a large voltage is applied to the photoelectric conversion film. Therefore, the quantum efficiency increases. Examples of the inorganic material that can be used for the electron blocking film include calcium oxide, chromium oxide, copper chromium oxide, manganese oxide, cobalt oxide, nickel oxide, copper oxide, copper gallium oxide, copper strontium oxide, niobium oxide, molybdenum oxide, copper indium oxide, silver indium oxide, and iridium oxide.

### [Positive Hole Blocking Film]

A positive hole blocking film is an acceptor-property organic semiconductor material (a compound), and the n-type organic semiconductor described above can be used.

In addition, the positive hole blocking film may be formed with a plurality of films.

Examples of a method of producing a charge blocking film include a dry film formation method and a wet film formation method. Examples of the dry film formation method include a vapor deposition method and a sputtering method. The vapor deposition method may be any of a physical vapor deposition (PVD) method and a chemical vapor deposition (CVD) method, and the physical vapor deposition method such as a vacuum vapor deposition method is preferable. Examples of the wet film formation method include an ink jet method, a spray method, a nozzle printing method, a spin coating method, a dip coating method, a casting method, a die coating method, a roll coating method, a bar coating method, and a gravure coating method, and an ink jet method is preferable from the viewpoint of high accuracy patterning.

Each film thickness of the charge blocking films (the electron blocking film and the positive hole blocking film) is preferably 3 to 200 nm, more preferably 5 to 100 nm, and still more preferably 5 to 30 nm.

### [Substrate]

The photoelectric conversion element may further include a substrate.

Examples of the substrate include a semiconductor substrate, a glass substrate, and a plastic substrate.

As a position of the substrate, in general, the conductive film, the photoelectric conversion film, and the transparent conductive film are laminated on the substrate in this order.

### [Sealing Layer]

The photoelectric conversion element may further include a sealing layer.

The performance of the photoelectric conversion material may deteriorate noticeably due to the presence of deterioration factors such as water molecules. The deterioration can be prevented by coating and sealing the entirety of the photoelectric conversion film with the sealing layer such as diamond-like carbon (DLC) or ceramics such as metal oxide, metal nitride, or metal nitride oxide which are dense and into which water molecules do not permeate.

Examples of the sealing layer include layers described in paragraphs [0210] to [0215] of JP2011-082508A, the contents of which are incorporated herein by reference.

### [Imaging Element]

An example of the application of the photoelectric conversion element includes an imaging element.

The imaging element is an element that converts optical information of an image into an electric signal. In general, a plurality of the photoelectric conversion elements are arranged in a matrix on the same plane, and an optical signal is converted into an electric signal in each photoelectric conversion element (pixel) to sequentially output the electric signal to the outside of the imaging element for each pixel. Therefore, each pixel is formed of one or more photoelectric conversion elements and one or more transistors.

### [Optical Sensor]

Examples of another application of the photoelectric conversion element include the photoelectric cell and the optical sensor, but the photoelectric conversion element of the embodiment of the invention is preferably used as the optical sensor. The photoelectric conversion element may be used alone as the optical sensor. Alternately, the photoelectric conversion element may be used as a line sensor in which the photoelectric conversion elements are linearly arranged or as a two-dimensional sensor in which the photoelectric conversion elements are arranged on a plane.

### [Compound]

The present invention further includes the invention of compounds. The compound according to the embodiment of the present invention is the specific compound.

### Examples

Hereinafter, the present invention will be described in more detail based on Examples.

The materials, the amounts and proportions of the materials used, the details of treatments, the procedure of treatments, and the like shown in the following Examples can be appropriately modified as long as the gist of the present invention is maintained. Therefore, the scope of the present invention should not be construed as being limited to Examples shown below.

### [Materials Used for Photoelectric Conversion Film]

### [Synthesis of Compound (1-4)]

Compound (1-4) was synthesized according to the following scheme.

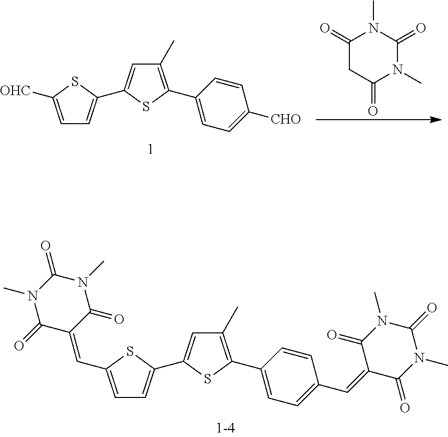

Compound (1) (500 mg), 1,3-dimethylbarbituric acid (manufactured by Tokyo Chemical Industry Co., Ltd., 600 mg), toluene (20 mL), and piperidine (32 L) were charged into a 100 mL eggplant flask, and the mixture was allowed to react at 80° C. for 3 hours under a nitrogen atmosphere. The precipitated solid was separated by filtration, and then washed with dimethylacetamide (DMAc) and tetrahydrofuran (THF) in this order. The obtained solid was purified by sublimation to obtain 568 mg of Compound (1-4) (yield of 60%). Since Compound (1-4) had low solubility, the structure thereof was confirmed by LDI-MS.

LDI-MS (Compound (1-4)): 588 (M⁺)

### [Synthesis of Compound (1)]

In addition, Compound (1) was synthesized according to the following scheme.

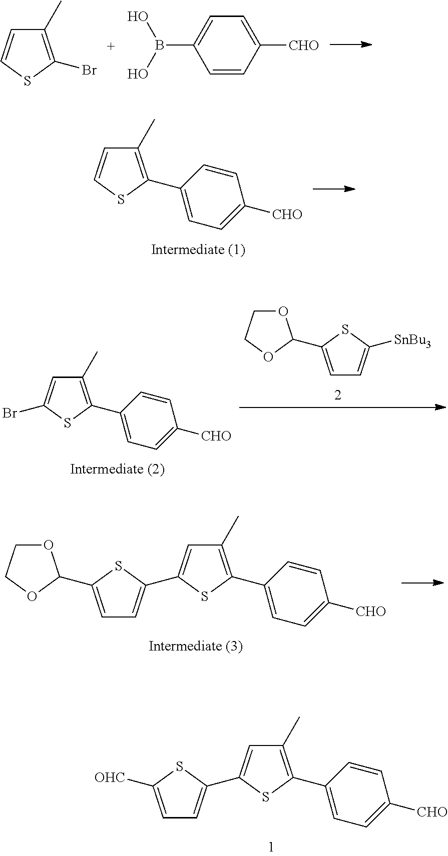

### <Synthesis of Intermediate (1)>

2-Bromo-3-methylthiophene (7.50 g), 4-formylphenylboronic acid (7.62 g), potassium carbonate (11.7 g), 4-methyltetrahydropyran (225 mL), water (75 mL), and (2-dicyclohexylphosphino-2′,6′-dimethoxybiphenyl)[2-(2′-amino-1,1′-biphenyl)]palladium(II) methanesulfonate (SPhos Pd G3, 0.992 g) were charged into a 500 mL three-neck flask, and the mixture was allowed to react at 100° C. for 1.5 hours under a nitrogen atmosphere. The obtained reaction solution was filtered through Celite, then the reaction solution was separated, and an organic layer was collected. The obtained organic layer was washed with 10% saline solution, then dried with sodium sulfate, and the solvent was further removed. The obtained crude product was purified by silica gel column chromatography (eluent: hexane/ethyl acetate=7/3), thereby obtaining 8.57 g of Intermediate (1) (yield of 82%).

### <Synthesis of Intermediate (2)>

Intermediate (1) (6.93 g) and N,N-dimethylformamide (DMF, 140 mL) were charged into a 300 mL three-neck flask, and the temperature was lowered to 10° C. or more. N-Bromosuccinimide (6.57 g) was further added thereto, and the mixture was allowed to react at room temperature for 2 hours. DMF (35 mL) and water (105 mL) were added to the obtained reaction solution, and the precipitated solid was separated by filtration. The obtained solid was recrystallized from dichloromethane and methanol to obtain 6.57 g of Intermediate (2) (yield of 70%).

### <Synthesis of Intermediate (3)>

Intermediate (2) (5.00 g), Compound (2) (9.60 g), DMF (100 mL), and tetrakis(triphenylphosphine) palladium(0) (0.616 g) were added to a 300 mL three-neck flask, and the mixture was allowed to react at 110° C. for 2 hours under a nitrogen atmosphere. Water (200 mL) was added to the obtained reaction solution, and the precipitated solid was separated by filtration. The obtained crude product was purified by silica gel column chromatography (eluent: hexane/ethyl acetate=7/3), thereby obtaining 5.81 g of Intermediate (3) (yield of 92%).

### <Synthesis of Compound (1)>

Intermediate (3) (5.81 g), THF (87 mL), and 10% hydrochloric acid (29 mL) were charged into a 300 mL three-neck flask, and the mixture was allowed to react at 70° C. for 15 minutes. Water (105 mL) was added to the obtained reaction solution, and the precipitated solid was separated by filtration. The obtained solid was recrystallized from dichloromethane and methanol to obtain 4.27 g of Compound (1) (yield of 84%).

The specific compound and the comparative compound, which were used in the photoelectric conversion film other than Compound (1-4) were synthesized with reference to the synthesis method of Compound (1-4).

Hereinafter, each material used in the photoelectric conversion film is shown. Compound (1-1) to Compound (1-40) and Compound (2-1) to Compound (2-10) correspond to specific compounds, and Compounds (C-1) to Compound (C-5) correspond to comparative compounds.

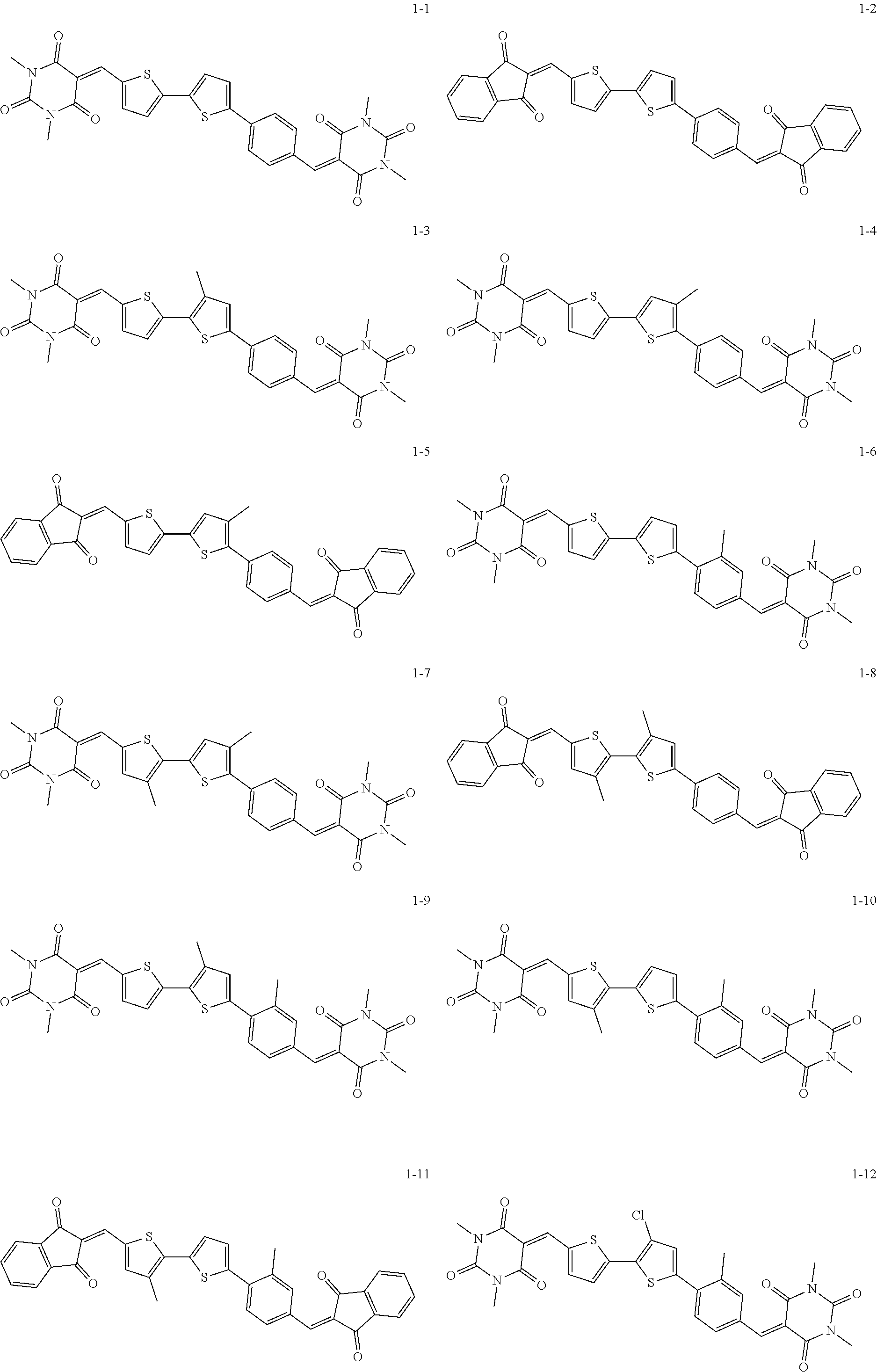

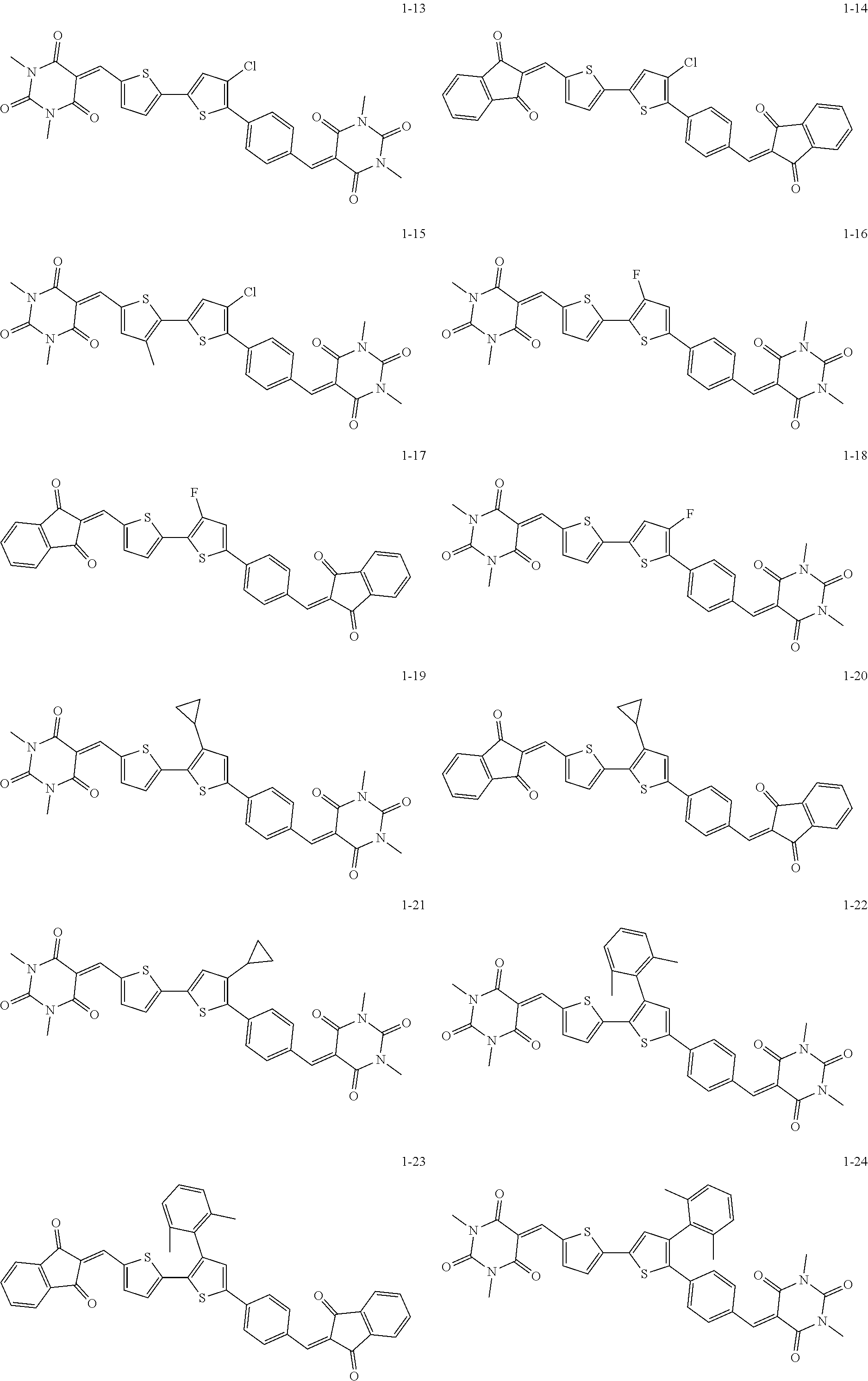

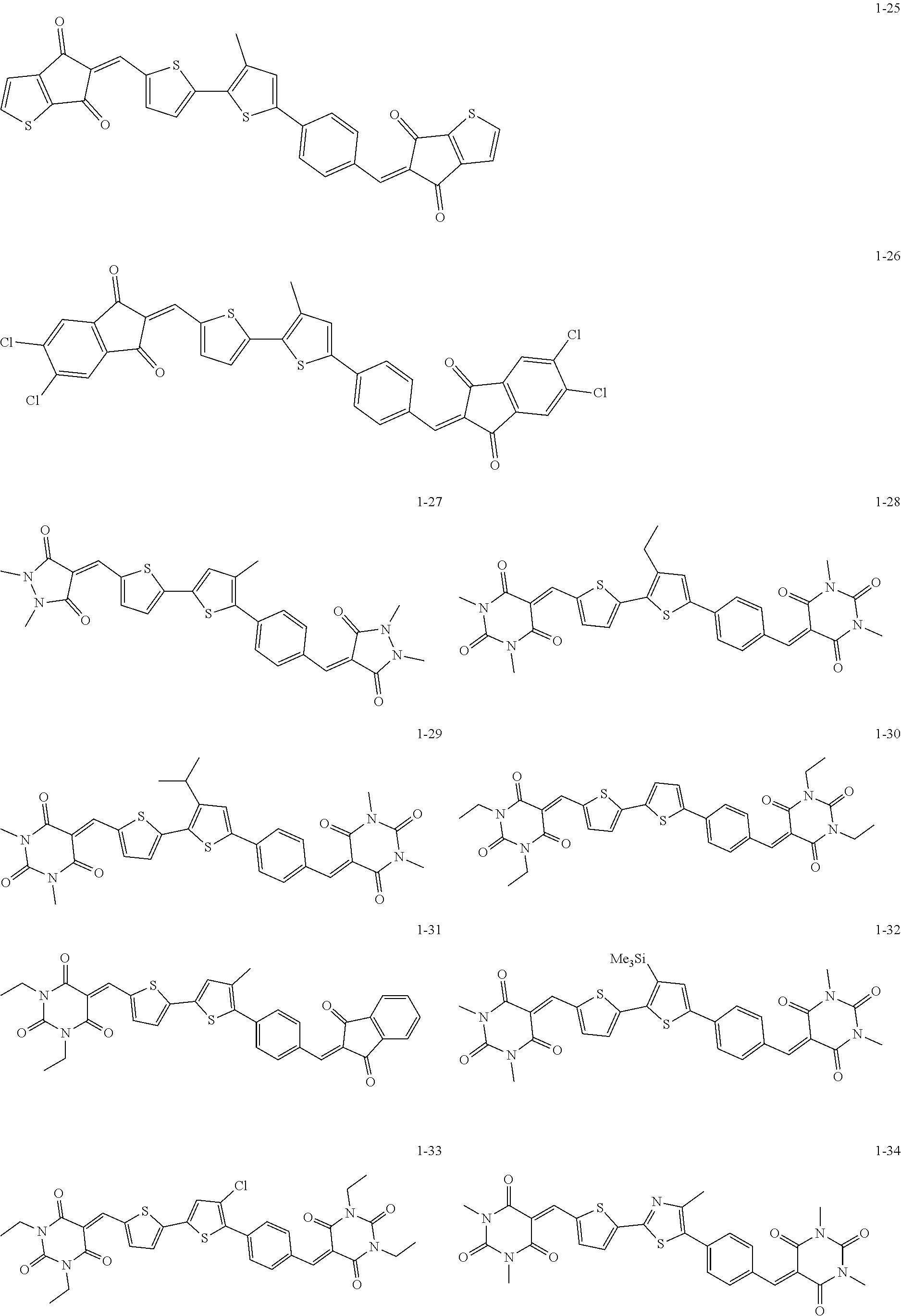

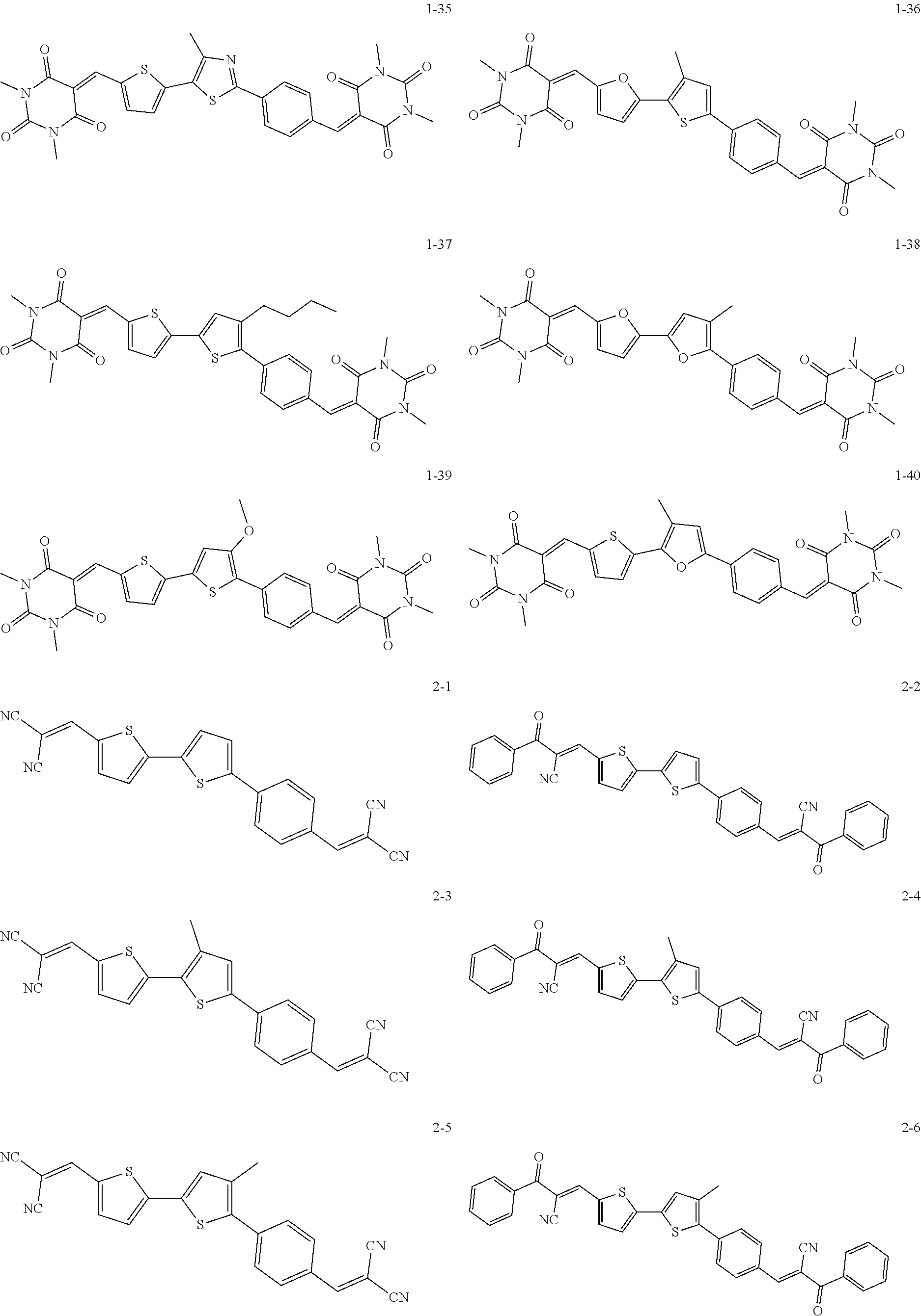

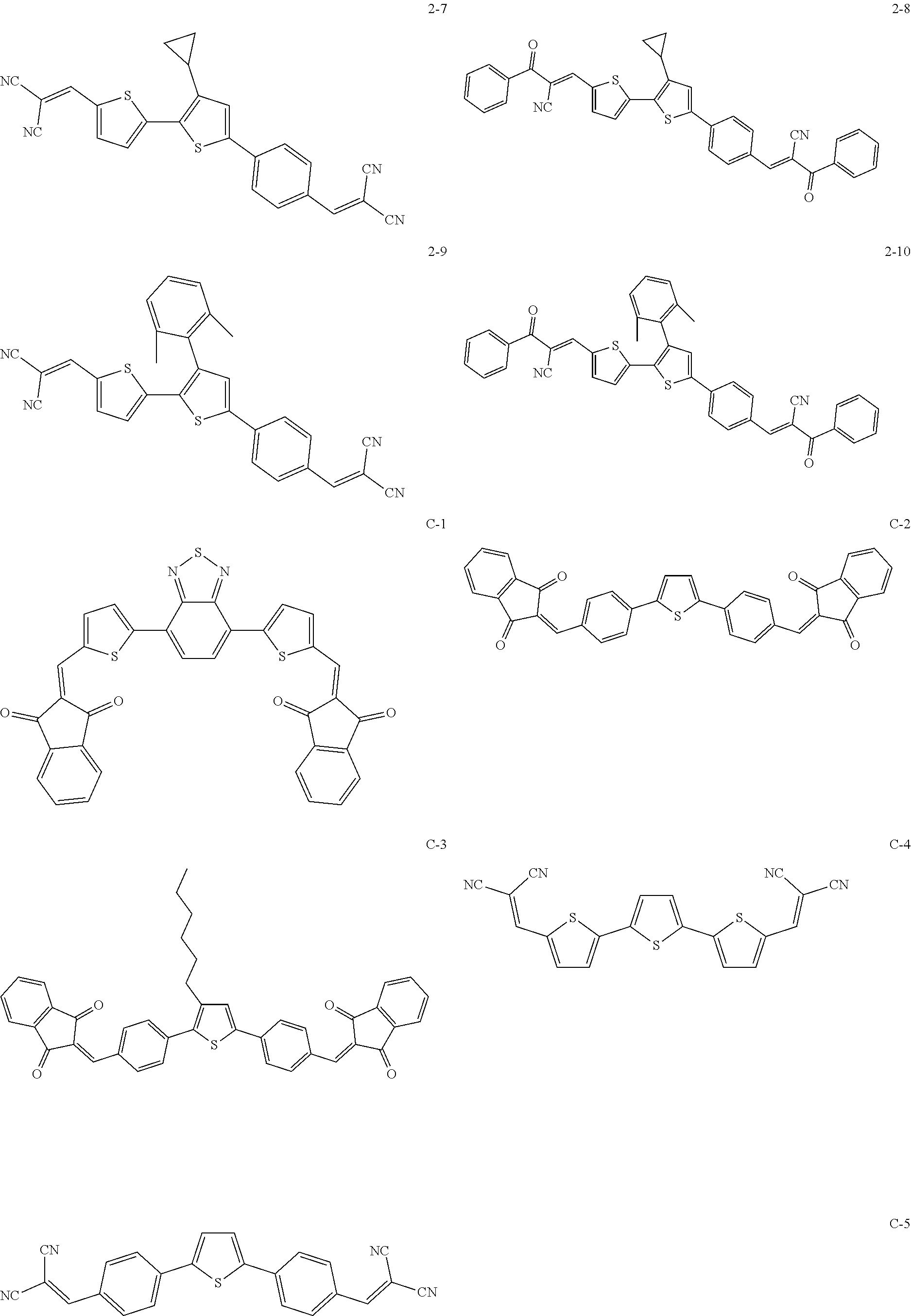

[n-Type Organic Semiconductor]
C₆₀: fullerene (C₆₀)
   [p-Type Organic Semiconductor]

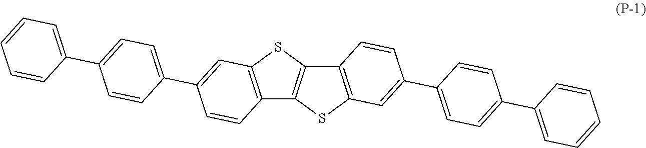 [SMILES: c1ccc(-c2ccc(-c3ccc4c(c3)sc3c5ccc(-c6ccc(-c7ccccc7)cc6)cc5sc43)cc2)cc1]

### [Coloring Agent]

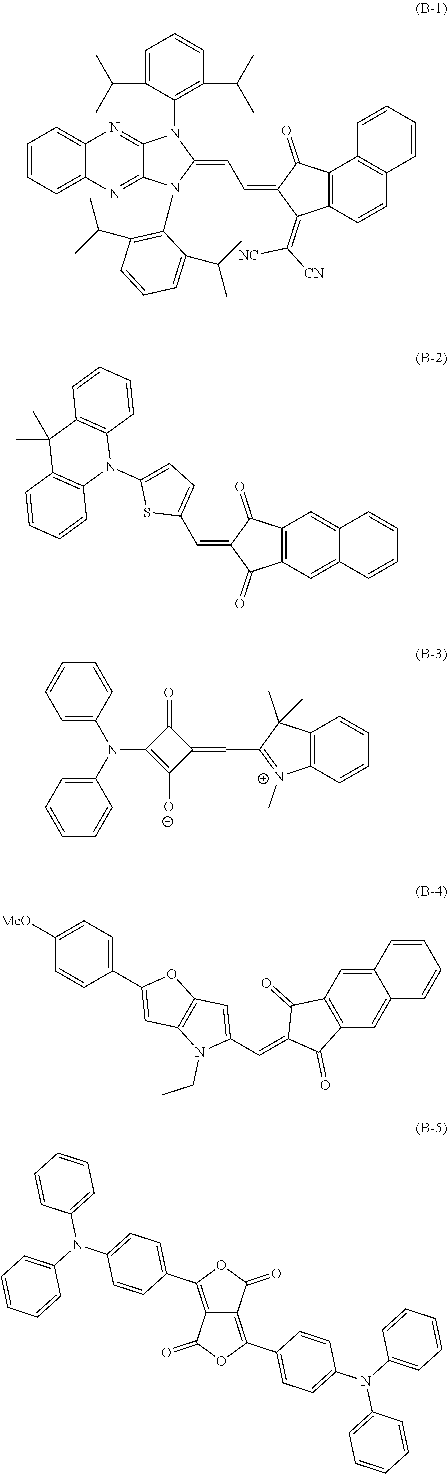 [SMILES: CC(C)c1cccc(C(C)C)c1N1C(=C/C=C2\C(=O)c3c(ccc4ccccc34)C2=C(C#N)C#N)N(c2c(C(C)C)cccc2C(C)C)c2nc3ccccc3nc21 ; CC1(C)c2ccccc2N(c2ccc(C=C3C(=O)c4cc5ccccc5cc4C3=O)s2)c2ccccc21 ; C[N+]1=C(C=C2C(=O)C(N(c3ccccc3)c3ccccc3)=C2[O-])C(C)(C)c2ccccc21 ; CCn1c(C=C2C(=O)c3cc4ccccc4cc3C2=O)cc2oc(-c3ccc(OC)cc3)cc21 ; O=C1OC(c2ccc(N(c3ccccc3)c3ccccc3)cc2)=C2C(=O)OC(c3ccc(N(c4ccccc4)c4ccccc4)cc3)=C12]

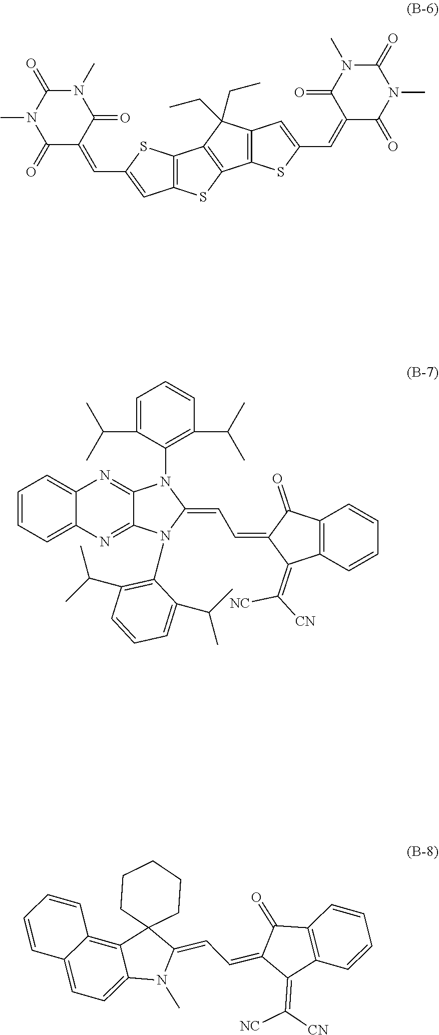

### [Evaluation]

### [Test X]

### <Production of Photoelectric Conversion Element>

The photoelectric conversion element of the form illustrated in FIG. 2 was produced using the obtained compounds. Here, the photoelectric conversion element includes a lower electrode 11, an electron blocking film 16A, a photoelectric conversion film 12, a positive hole blocking film 161B, and an upper electrode 15.

Specifically, an amorphous ITO was formed into a film on a glass substrate by a sputtering method to form the lower electrode 11 (thickness: 30 nm). Furthermore, a compound (EB-1) was formed into a film on the lower electrode 11 by a vacuum thermal vapor deposition method to form the electron blocking film 16A (thickness: 30 nm).

Subsequently, in a state where the temperature of the glass substrate was controlled to 25° C., each specific compound or each comparative compound shown in Tables 1 and 2, the n-type organic semiconductor (fullerene (C₆₀)), and the p-type organic semiconductor (Compound (P-1)) were co-vapor deposited on the electron blocking film 16A by a vacuum vapor deposition method, each to be 80 nm in terms of a single layer, thereby forming a film. As a result, the photoelectric conversion film 12 having a bulk hetero structure with a wavelength of 240 nm was formed.

Furthermore, a compound (EB-2) was vapor-deposited on the photoelectric conversion film 12 to form the positive hole blocking film 16B (thickness: 10 nm). Amorphous ITO was formed into a film on the positive hole blocking film 16B by a sputtering method to form the upper electrode 15 (the transparent conductive film) (thickness: 10 nm). After the SiO film was formed as the sealing layer on the upper electrode 15 by a vacuum vapor deposition method, an aluminum oxide (Al₂O₃) layer was formed thereon by an atomic layer chemical vapor deposition (ALCVD) method. The obtained laminate was heated in a glove box at 150° C. for 30 minutes to obtain a photoelectric conversion element.

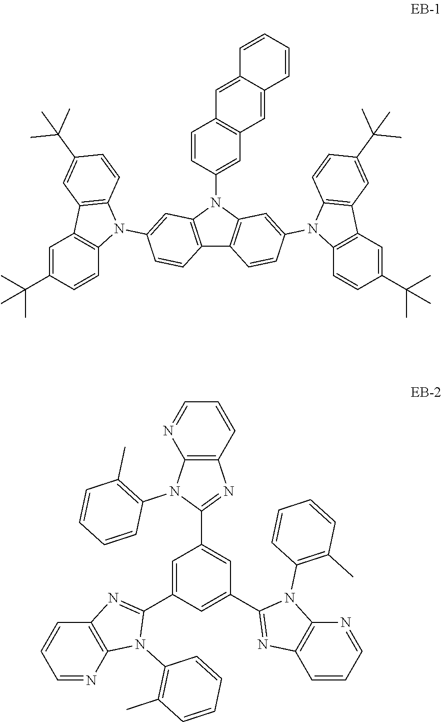 [SMILES: CC(C)(C)c1ccc2c(c1)c1cc(C(C)(C)C)ccc1n2-c1ccc2c3ccc(-n4c5ccc(C(C)(C)C)cc5c5cc(C(C)(C)C)ccc54)cc3n(-c3ccc4cc5ccccc5cc4c3)c2c1 ; Cc1ccccc1-n1c(-c2cc(-c3nc4cccnc4n3-c3ccccc3C)cc(-c3nc4cccnc4n3-c3ccccc3C)c2)nc2cccnc21]

### <Dark Current>

The dark current of each of the obtained photoelectric conversion elements was measured by the following method. A voltage was applied to the lower electrode and the upper electrode of each of the photoelectric conversion elements to have an electric field strength of 2.5×10⁵ V/cm and current values (dark current) in a dark place were measured. As a result, it was confirmed that all of the photoelectric conversion elements had a dark current of 50 nA/cm² or less, which indicates that all of the photoelectric conversion elements had a sufficiently low dark current.

### <Quantum Efficiency>

The quantum efficiency of each of the obtained photoelectric conversion elements was measured by the following method.

A voltage was applied to each photoelectric conversion element such that the electric field strength was 2.0×10⁵ V/cm, and then light was emitted from the upper electrode (transparent conductive film) side to evaluate the quantum efficiency (photoelectric conversion efficiency) at a wavelength of 460 nm. The quantum efficiency was obtained according to Expression (S1).

Expression (S1): quantum efficiency (relative ratio)=(quantum efficiency at wavelength of 460 nm in each of Examples or each of Comparative Examples)/(quantum efficiency at wavelength of 460 nm in the reference example)
A: quantum efficiency of 1.6 or more.
B: quantum efficiency of 1.2 or more and less than 1.6.
C: quantum efficiency of 0.8 or more and less than 1.2.
D: quantum efficiency of 0.4 or more and less than 0.8.
E: quantum efficiency of less than 0.4.

In Examples 1-1 to 1-40 and Comparative Examples 1-1 to 1-3 shown in Table 1, Example 1-1 was adopted as the above-described reference example in Formula (S1), and in Examples 1-41 to 1-51 and Comparative Examples 1-4 and 1-5 shown in Table 2, Example 1-41 was adopted as the above-described reference example in Formula (S1).

### <Response Speed (Responsiveness)>

The response speed of each obtained photoelectric conversion element was evaluated by the following method.

A voltage was applied to the photoelectric conversion element to have a strength of 2.0×10⁵ V/cm. Thereafter, a light emitting diode (LED) was turned on for an instant to emit light from the upper electrode (transparent conductive film) side, a photocurrent at this time at a wavelength of 460 nm was measured with an oscilloscope, a rise time until the signal strength rose from 0% to 97% was measured, and the relative response speed was evaluated according to Expression (S2).

Expression (S2): relative response speed=(rise time at a wavelength of 460 nm in each of Examples or each of Comparative Examples)/(rise time at a wavelength of 460 nm in reference example)
A: relative response speed of less than 0.5
B: relative response speed of 0.5 or more and less than 1.0.
C: relative response speed of 1.0 or more and less than 1.5.
D: relative response speed of 1.5 or more and less than 2.0.
E: relative response speed of 2.0 or more.

In Examples 1-1 to 1-40 and Comparative Examples 1-1 to 1-3 shown in Table 1, Example 1-1 was adopted as the above-described reference example in Formula (S2), and in Examples 1-41 to 1-51 and Comparative Examples 1-4 and 1-5 shown in Table 2, Example 1-41 was adopted as the above-described reference example in Formula (S2).

### <Electric Field Strength Dependence of Response Speed>

For each of the obtained photoelectric conversion elements, the electric field strength dependence of the response speed was evaluated by the following method.

The response speed at 7.5×10⁴ V/cm was measured by the same procedure as in the evaluation of the above-described <Response speed>, except that the voltage applied to each photoelectric conversion element was changed to 7.5×10⁴ V/cm.

The evaluation was performed by obtaining the electric field strength dependence of the response speed according to Expression (S3).

Expression (S3): electric field strength dependence of response speed=(rise time at 7.5×10⁴ V/cm at a wavelength of 460 nm in each of Examples or each of Comparative Examples)/(rise time at 2.0×10⁵ V/cm at a wavelength of 460 nm in each of Examples or each of Comparative Examples)

In Expression (S3), each photoelectric conversion element in the numerator and the denominator are the same. For example, in the case of Example 1-1, the rise time of the photoelectric conversion efficiency at 7.5×10⁴ V/cm at a wavelength of 460 nm in Example 1-1 and the rise time of the photoelectric conversion efficiency at 2.0×10⁵ V/cm at a wavelength of 460 nm in Example 1-1 are compared.
A: electric field strength dependence of the response speed of less than 2.0.
B: electric field strength dependence of the response speed of 2.0 or more and less than 3.0.
C: electric field strength dependence of the response speed of 3.0 or more and less than 4.0.
D: electric field strength dependence of the response speed of 4.0 or more and less than 5.0.
E: electric field strength dependence of the response speed of 5.0 or more.

The evaluation results of Test X are shown in Tables 1 and 2.

Each notation in Tables 1 and 2 represents the following.

In the column of “Formulae (1-1) to (1-7)”, a case where the specific compound was a compound represented by any of Formula (1-1) to Formula (1-7) was defined as “A”, and the other cases were defined as “B”.

The column of “Rs”, a case where Rs in Formulae (1-1) to (1-7) was selected from the group consisting of a linear aliphatic hydrocarbon group having 1 to 3 carbon atoms, which may have a halogen atom, a branched aliphatic hydrocarbon group having 3 to 5 carbon atoms, which may have a halogen atom, a cyclic aliphatic hydrocarbon group having 3 to 8 carbon atoms, which may have a halogen atom, an aromatic ring group having 4 to 10 carbon atoms, which may have a substituent, and an alkoxy group having 1 to 3 carbon atoms, was defined as “A” and the other cases were defined as “B”.

The column of “A¹, A²”, a case where A¹ and A² in Formula (1) were each the group represented by Formula (A-1) was defined as “A-1”, and a case where A¹ and A² in Formula (1) were each the group represented by Formula (A-2) was defined as “A-2”.

In the column of “Formula (C-1), Formula (C-2)”, a case where the group represented by Formula (A-1) in Formula (1) was the group represented by Formula (C-1) or Formula (C-2) was defined as “A”, and the other cases were defined as “B”.

In the column of “X¹, X²”, a case where X¹ and X² in Formula (1) were each sulfur atoms was defined as “A”, and the other cases were defined as “B”.

TABLE 1

| | | | | | | | Evaluation | | |
|---|---|---|---|---|---|---|---|---|---|
| | Specific compound or comparative compound | | | | | | | | Electric field |
| | | Formula (1-1) | | | | | | | strength |
| | | to | | A¹, | Formula (C-1), | X¹, | Quantum | Response | dependence of |
| | Compound | Formula (1-7) | Rs | A² | Formula (C-2) | X² | efficiency | speed | response speed |
| Example 1-1 | 1-1 | B | — | A-1 | A | A | C | C | B |
| Example 1-2 | 1-2 | B | — | A-1 | A | A | C | C | B |
| Example 1-3 | 1-3 | A | A | A-1 | A | A | A | A | A |
| Example 1-4 | 1-4 | A | A | A-1 | A | A | A | A | A |
| Example 1-5 | 1-5 | A | A | A-1 | A | A | B | A | A |
| Example 1-6 | 1-6 | A | A | A-1 | A | A | A | A | A |
| Example 1-7 | 1-7 | A | A | A-1 | A | A | A | A | A |
| Example 1-8 | 1-8 | A | A | A-1 | A | A | B | A | A |
| Example 1-9 | 1-9 | A | A | A-1 | A | A | A | A | A |
| Example 1-10 | 1-10 | A | A | A-1 | A | A | A | A | A |
| Example 1-11 | 1-11 | A | A | A-1 | A | A | B | A | A |
| Example 1-12 | 1-12 | B | — | A-1 | A | A | C | C | B |
| Example 1-13 | 1-13 | B | — | A-1 | A | A | C | C | B |
| Example 1-14 | 1-14 | B | — | A-1 | A | A | C | C | B |
| Example 1-15 | 1-15 | A | A | A-1 | A | A | A | A | A |
| Example 1-16 | 1-16 | B | — | A-1 | A | A | C | C | B |
| Example 1-17 | 1-17 | B | — | A-1 | A | A | C | C | B |
| Example 1-18 | 1-18 | B | — | A-1 | A | A | C | C | B |
| Example 1-19 | 1-19 | A | A | A-1 | A | A | A | A | A |
| Example 1-20 | 1-20 | A | A | A-1 | A | A | B | A | A |
| Example 1-21 | 1-21 | A | A | A-1 | A | A | A | A | A |
| Example 1-22 | 1-22 | A | A | A-1 | A | A | A | A | A |
| Example 1-23 | 1-23 | A | A | A-1 | A | A | B | A | A |
| Example 1-24 | 1-24 | A | A | A-1 | A | A | A | A | A |
| Example 1-25 | 1-25 | A | A | A-1 | A | A | B | A | A |
| Example 1-26 | 1-26 | A | A | A-1 | A | A | B | A | A |
| Example 1-27 | 1-27 | A | A | A-1 | B | A | C | B | B |
| Example 1-28 | 1-28 | A | A | A-1 | A | A | A | A | A |
| Example 1-29 | 1-29 | A | A | A-1 | A | A | A | A | A |
| Example 1-30 | 1-30 | B | — | A-1 | A | A | C | C | B |
| Example 1-31 | 1-31 | A | A | A-1 | A | A | A | A | A |
| Example 1-32 | 1-32 | B | — | A-1 | A | A | C | C | B |
| Example 1-33 | 1-33 | B | — | A-1 | A | A | C | C | B |
| Example 1-34 | 1-34 | A | A | A-1 | A | A | A | A | A |
| Example 1-35 | 1-35 | A | A | A-1 | A | A | A | A | A |
| Example 1-36 | 1-36 | A | A | A-1 | A | B | A | B | B |
| Example 1-37 | 1-37 | A | B | A-1 | A | A | C | C | B |
| Example 1-38 | 1-38 | A | A | A-1 | A | B | A | B | B |
| Example 1-39 | 1-39 | A | A | A-1 | A | A | A | A | A |
| Example 1-40 | 1-40 | A | A | A-1 | A | B | A | B | B |
| Comparative | C-1 | — | — | — | — | — | E | E | E |
| Example 1-1 | | | | | | | | | |
| Comparative | C-2 | — | — | — | — | — | D | C | D |
| Example 1-2 | | | | | | | | | |
| Comparative | C-3 | — | — | — | — | — | C | D | C |
| Example 1-3 | | | | | | | | | |

TABLE 2

| | | | | | | | Evaluation | | |
|---|---|---|---|---|---|---|---|---|---|
| | Specific compound or comparative compound | | | | | | | | Electric field |
| | | Formula (1-1) | | | | | | | strength |
| | | to | | A¹, | Formula (C-1), | X¹, | Quantum | Response | dependence of |
| | Compound | Formula (1-7) | Rs | A² | Formula (C-2) | X² | efficiency | speed | response speed |
| Example 1-41 | 2-1 | B | — | A-2 | — | A | C | C | C |
| Example 1-42 | 2-2 | B | — | A-2 | — | A | C | C | B |
| Example 1-43 | 2-3 | A | A | A-2 | — | A | B | B | B |
| Example 1-44 | 2-4 | A | A | A-2 | — | A | B | B | B |
| Example 1-45 | 2-5 | A | A | A-2 | — | A | B | B | B |
| Example 1-46 | 2-6 | A | A | A-2 | — | A | B | B | B |
| Example 1-47 | 2-7 | A | A | A-2 | — | A | B | B | B |
| Example 1-48 | 2-8 | A | A | A-2 | — | A | B | B | B |
| Example 1-49 | 2-9 | A | A | A-2 | — | A | B | B | B |
| Example 1-50 | 2-10 | A | A | A-2 | — | A | B | B | B |
| Example 1-51 | 1-4 | A | A | A-1 | A | A | A | A | A |
| Comparative | C-4 | — | — | — | — | — | E | E | E |
| Example 1-4 | | | | | | | | | |
| Comparative | C-5 | — | — | — | — | — | D | D | D |
| Example 1-5 | | | | | | | | | |

From the results shown in the table, it was confirmed that the desired effects were obtained in the photoelectric conversion element according to the embodiment of the present invention.

From the comparison between Examples 1-1, 1-2, 1-13, and 1-14 and Examples 1-4, 1-5, 1-19, 1-20, 1-22, and 1-23, and the like, it was found that, in a case where the specific compound was the compound represented by any of Formulae (1-1) to (1-7), the effect of the present invention was more excellent.

From the comparison between Example 1-37 and Example 1-4, and the like, it was found that, in a case where Rs in the compound represented by Formula (1) (specific compound) was selected from the group consisting of the linear aliphatic hydrocarbon group having 1 to 3 carbon atoms, which may have a halogen atom, the branched aliphatic hydrocarbon group having 3 to 5 carbon atoms, which may have a halogen atom, the cyclic aliphatic hydrocarbon group having 3 to 8 carbon atoms, which may have a halogen atom, the aromatic ring group having 4 to 10 carbon atoms, which may have a substituent, and the alkoxy group having 1 to 3 carbon atoms, the effect of the present invention was more excellent.

From the comparison between Examples 1-45 and 1-48 and Example 1-51, and the like, it was found that, in a case where A¹ and A² in the compound represented by Formula (1) (specific compound) were each the group represented by Formula (A-1), the effect of the present invention was more excellent.

From the comparison between Example 1-27 and Examples 1-4 and 1-5, and the like, it was found that, in a case where the group represented by Formula (A-1) was the group represented by Formula (C-1) or the group represented by Formula (C-2), the effect of the present invention was more excellent.

From the comparison between Example 1-36 and Example 1-4, and the like, it was found that, in a case where X¹ and X² in the compound represented by Formula (1) (specific compound) were each a sulfur atom, the effect of the present invention was more excellent.

### [Test Y]

### <Production of Photoelectric Conversion Element>

A photoelectric conversion film was formed by co-vapor depositing each of the specific compound or each of the comparative compound, the n-type organic semiconductor (fullerene (C₆₀)), the p-type organic semiconductor (Compound (P-1)), the above-described compounds, which were shown in Tables 3 and 4, and the coloring agent in a ratio of compound:coloring agent:p-type organic semiconductor:n-type organic semiconductor=1:1:2:2 in terms of a single layer by a vacuum deposition method, and a photoelectric conversion element of each of Examples and each of Comparative Examples was produced in the same manner for the other procedures as in Test X.

### <Dark Current>

The dark current was measured in the same manner as in Test X.

As a result, it was confirmed that all of the photoelectric conversion elements had a dark current of 50 nA/cm² or less, which indicates that all of the photoelectric conversion elements had a sufficiently low dark current.

### <Quantum Efficiency>

The quantum efficiency of each of the obtained photoelectric conversion elements was measured by the following method.

A voltage was applied to each photoelectric conversion element such that the electric field strength was 2.0×10⁵ V/cm, and then light was emitted from the upper electrode (transparent conductive film) side to evaluate the quantum efficiency at a wavelength of 460 nm or a wavelength of 600 nm. The quantum efficiency was obtained according to Expression (S4).

Expression (S4): quantum efficiency (relative ratio)=(quantum efficiency of each of Examples or each of Comparative Examples at wavelength of 460 nm or wavelength of 600 nm)/(quantum efficiency of the reference example at wavelength of 460 nm or wavelength of 600 nm)
A: quantum efficiency of 1.6 or more.
B: quantum efficiency of 1.2 or more and less than 1.6.
C: quantum efficiency of 0.8 or more and less than 1.2.
D: quantum efficiency of 0.4 or more and less than 0.8.
E: quantum efficiency of less than 0.4.

In Examples 2-1 to 2-20 and Comparative Examples 2-1 to 2-3 shown in Table 3, as the above-described reference example in Formula (S4), Example 2-1 was adopted for the quantum efficiency at a wavelength of 460 nm, and Comparative Example 2-2 was adopted for the quantum efficiency at a wavelength of 600 nm.

In addition, in Examples 2-21 to 2-27 and Comparative Examples 2-4 and 2-5 shown in Table 4, as the above-described reference example in Formula (S4), Example 2-21 was adopted for the quantum efficiency at a wavelength of 460 nm, and Comparative Example 2-5 was adopted for the quantum efficiency at a wavelength of 600 nm.

### <Response Speed>

The response speed of each obtained photoelectric conversion element was evaluated by the following method.

A voltage was applied to the photoelectric conversion element to have a strength of 2.0×10⁵ V/cm. Thereafter, the LED was turned on for an instant to emit light from the upper electrode (transparent conductive film) side, the photocurrent at this time at a wavelength of 460 nm or a wavelength of 600 nm was measured with an oscilloscope, a rise time until the signal intensity rose from 0% to 97% signal intensity was measured, and the relative response speed was evaluated according to Expression (S5). In a case of obtaining the relative response speed, the numerator and the denominator are the rise times at the same wavelength.

Expression (S5): relative response speed=(rise time at a wavelength of 460 nm or wavelength of 600 nm in each of Examples or each of Comparative Examples)/(rise time at a wavelength of 460 nm or wavelength of 600 nm in reference example)
A: relative response speed of less than 0.5
B: relative response speed of 0.5 or more and less than 1.0.
C: relative response speed of 1.0 or more and less than 1.5.
D: relative response speed of 1.5 or more and less than 2.0.
E: relative response speed of 2.0 or more.

In Examples 2-1 to 2-20 and Comparative Examples 2-1 to 2-3 shown in Table 3, as the above-described reference example in Formula (S5), Example 2-1 was adopted for the relative response speed at a wavelength of 460 nm, and Comparative Example 2-2 was adopted for the relative response speed at a wavelength of 600 nm.

In addition, in Examples 2-21 to 2-27 and Comparative Examples 2-4 and 2-5 shown in Table 4, as the above-described reference example in Formula (S5), Example 2-21 was adopted for the relative response speed at a wavelength of 460 nm, and Comparative Example 2-5 was adopted for the relative response speed at a wavelength of 600 nm.

### <Electric Field Strength Dependence of Response Speed>

For each of the obtained photoelectric conversion elements, the electric field strength dependence of the response speed was evaluated by the following method.

The response speed at 7.5×10⁴ V/cm was measured by the same procedure as in the evaluation of the response speed of Test Y, except that the voltage applied to each photoelectric conversion element was changed to 7.5×10⁴ V/cm.

The evaluation was performed by obtaining the electric field strength dependence of the response speed according to Expression (S6).

Expression (S6): electric field strength dependence of response speed=(rise time at 7.5×10⁴ V/cm at each wavelength in each of Examples or each of Comparative Examples)/(rise time at 2.0×10⁵ V/cm at each wavelength in each of Examples or each of Comparative Examples)

In Expression (S6), each photoelectric conversion element in the numerator and the denominator are the same.
A: electric field strength dependence of the response speed of less than 2.0.
B: electric field strength dependence of the response speed of 2.0 or more and less than 3.0.
C: electric field strength dependence of the response speed of 3.0 or more and less than 4.0.
D: electric field strength dependence of the response speed of 4.0 or more and less than 5.0.
E: electric field strength dependence of the response speed of 5.0 or more.

Tables 3 and 4 show the evaluation results of Test Y.

Each notation in Tables 3 and 4 is as described above for each notation in Tables 1 and 2.

| | Specific compound or comparative compound | | | | | | | Evaluation |
|---|---|---|---|---|---|---|---|---|
| | | Formula (1-1) | | | | | Combination | Quantum |
| | | to | | A¹, | Formula (C-1), | X¹, | coloring | efficiency |
| | Compound | Formula (1-7) | Rs | A² | Formula (C-2) | X² | agent | (460 nm) |
| Example 2-1 | 1-1 | B | — | A-1 | A | A | B-1 | C |
| Example 2-2 | 1-1 | B | — | A-1 | A | A | B-2 | C |
| Example 2-3 | 1-1 | B | — | A-1 | A | A | B-3 | C |
| Example 2-4 | 1-1 | B | — | A-1 | A | A | B-4 | C |
| Example 2-5 | 1-1 | B | — | A-1 | A | A | B-5 | C |
| Example 2-6 | 1-1 | B | — | A-1 | A | A | B-6 | C |
| Example 2-7 | 1-1 | B | — | A-1 | A | A | B-7 | C |
| Example 2-8 | 1-1 | B | — | A-1 | A | A | B-8 | C |
| Example 2-9 | 1-2 | B | — | A-1 | A | A | B-8 | C |
| Example 2-10 | 1-4 | A | A | A-1 | A | A | B-8 | A |
| Example 2-11 | 1-8 | A | A | A-1 | A | A | B-8 | B |
| Example 2-12 | 1-9 | A | A | A-1 | A | A | B-8 | A |
| Example 2-13 | 1-13 | B | — | A-1 | A | A | B-8 | C |
| Example 2-14 | 1-19 | A | A | A-1 | A | A | B-8 | A |
| Example 2-15 | 1-20 | A | A | A-1 | A | A | B-8 | B |
| Example 2-16 | 1-23 | A | A | A-1 | A | A | B-8 | B |
| Example 2-17 | 1-28 | A | A | A-1 | A | A | B-8 | A |
| Example 2-18 | 1-29 | A | A | A-1 | A | A | B-8 | A |
| Example 2-19 | 1-34 | A | A | A-1 | A | A | B-8 | A |
| Example 2-20 | 1-37 | A | B | A-1 | A | A | B-8 | C |
| Comparative | C-1 | — | — | — | — | — | B-8 | E |
| Example 2-1 | | | | | | | | |
| Comparative | C-2 | — | — | — | — | — | B-8 | D |
| Example 2-2 | | | | | | | | |
| Comparative | C-3 | — | — | — | — | — | B-8 | C |
| Example 2-3 | | | | | | | | |

| | | | | Electric field | Electric field |
|---|---|---|---|---|---|
| | | | | strength | strength |
| | Quantum | Response | Response | dependence of | dependence of |
| | efficiency | speed | speed | response speed | response speed |
| | (600 nm) | (460 nm) | (600 nm) | (460 nm) | (600 nm) |
| Example 2-1 | B | C | B | B | B |
| Example 2-2 | B | C | B | B | B |
| Example 2-3 | B | C | B | B | B |
| Example 2-4 | B | C | B | B | B |
| Example 2-5 | B | C | B | B | B |
| Example 2-6 | B | C | B | B | B |
| Example 2-7 | B | C | B | B | B |
| Example 2-8 | B | C | B | B | B |
| Example 2-9 | B | C | B | B | B |
| Example 2-10 | A | A | A | A | A |
| Example 2-11 | A | A | A | A | A |
| Example 2-12 | A | A | A | A | A |
| Example 2-13 | B | C | B | B | B |
| Example 2-14 | A | A | A | A | A |
| Example 2-15 | A | A | A | A | A |
| Example 2-16 | A | A | A | A | A |
| Example 2-17 | A | A | A | A | A |
| Example 2-18 | A | A | A | A | A |
| Example 2-19 | A | A | A | A | A |
| Example 2-20 | B | C | B | B | B |
| Comparative | D | E | D | E | D |
| Example 2-1 | | | | | |
| Comparative | C | C | C | D | C |
| Example 2-2 | | | | | |
| Comparative | C | D | C | C | C |
| Example 2-3 | | | | | |

| | Specific compound or comparative compound | | | | | | | Evaluation |
|---|---|---|---|---|---|---|---|---|
| | | Formula (1-1) | | | | | Combination | Quantum |
| | | to | | A¹, | Formula (C-1), | X¹, | coloring | efficiency |
| | Compound | Formula (1-7) | Rs | A² | Formula (C-2) | X² | agent | (460 mm) |
| Example 2-21 | 2-1 | B | — | A-2 | — | A | B-8 | C |
| Example 2-22 | 2-2 | B | — | A-2 | — | A | B-8 | C |
| Example 2-23 | 2-3 | A | A | A-2 | — | A | B-8 | B |
| Example 2-24 | 2-7 | A | A | A-2 | — | A | B-8 | B |
| Example 2-25 | 2-8 | A | A | A-2 | — | A | B-8 | B |
| Example 2-26 | 2-9 | A | A | A-2 | — | A | B-8 | B |
| Example 2-27 | 2-10 | A | A | A-2 | — | A | B-8 | B |
| Comparative | C-4 | — | — | — | — | — | B-8 | E |
| Example 2-4 | | | | | | | | |
| Comparative | C-5 | — | — | — | — | — | B-8 | D |
| Example 2-5 | | | | | | | | |

| | | | | Electric field | Electric field |
|---|---|---|---|---|---|
| | | | | strength | strength |
| | Quantum | Response | Response | dependence of | dependence of |
| | efficiency | speed | speed | response speed | response speed |
| | (600 nm) | (460 nm) | (600 nm) | (460 mm) | (600 mm) |
| Example 2-21 | B | C | B | C | B |
| Example 2-22 | B | C | B | B | A |
| Example 2-23 | A | B | A | B | A |
| Example 2-24 | A | B | A | B | A |
| Example 2-25 | A | B | A | B | A |
| Example 2-26 | A | B | A | B | A |
| Example 2-27 | A | B | A | B | A |
| Comparative | D | E | D | E | D |
| Example 2-4 | | | | | |
| Comparative | C | D | C | D | C |
| Example 2-5 | | | | | |

From the results shown in the table, it was confirmed that the desired effects were obtained in the photoelectric conversion element according to the embodiment of the present invention.

From the comparison between Examples 2-8, 2-9, and 2-13 and Examples 2-10 to 2-12, and the like, it was found that, in a case where the specific compound was the compound represented by any of Formulae (1-1) to (1-7), the effect of the present invention was more excellent. In addition, similarly from the comparison between Example 2-21 and Example 2-23, and the like, it was found that, in a case where the specific compound was the compound represented by any of Formulae (1-1) to (1-7), the effect of the present invention was more excellent.

From the comparison between Example 2-20 and Example 2-10, and the like, it was found that, in a case where Rs in the compound represented by Formula (1) (specific compound) was selected from the group consisting of the linear aliphatic hydrocarbon group having 1 to 3 carbon atoms, which may have a halogen atom, the branched aliphatic hydrocarbon group having 3 to 5 carbon atoms, which may have a halogen atom, the cyclic aliphatic hydrocarbon group having 3 to 8 carbon atoms, which may have a halogen atom, the aromatic ring group having 4 to 10 carbon atoms, which may have a substituent, and the alkoxy group having 1 to 3 carbon atoms, the effect of the present invention was more excellent.

### EXPLANATION OF REFERENCES

10a, 10b: photoelectric conversion element
11: conductive film (lower electrode)
12: photoelectric conversion film
15: transparent conductive film (upper electrode)
16A: electron blocking film
16B: positive hole blocking film

What is claimed is:

1. A photoelectric conversion element comprising, in the following order:
a conductive film;
a photoelectric conversion film; and
a transparent conductive film,
wherein the photoelectric conversion film contains a compound represented by Formula (1), 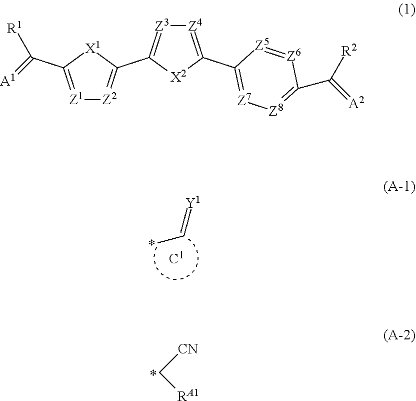
in Formula (1),
R¹ and R² each independently represent a hydrogen atom or a substituent,
X¹ and X² each independently represent a sulfur atom, an oxygen atom, or a selenium atom,
Z¹ to Z⁸ each independently represent —CR^{X1}═ or a nitrogen atom, in a case where adjacent two of Z¹ to Z⁸ are —CR^{X1}═, two R^{X1}'s may be bonded to each other to form a ring,
R^{X1} represents a hydrogen atom or a substituent, and
A¹ and A² each independently represent a group represented by Formula (A-1) or a group represented by Formula (A-2),
in Formulae (A-1) and (A-2), * represents a bonding position,
in Formula (A-1),
C¹ represents a ring which contains two or more carbon atoms and may have a substituent,
Y¹ represents a sulfur atom, an oxygen atom, ═NR^{X2}, or ═CR^{X3}R^{X4},
R^{X2} represents a hydrogen atom or a substituent,
R^{X3} and R^{X4} each independently represent a cyano group, —SO₂R^{X5}, —COOR^{X6}, or —COR^{X7}, and
R^{X5} to R^{X7} each independently represent an aliphatic hydrocarbon group which may have a substituent, an aromatic ring group which may have a substituent, or an aliphatic heterocyclic group which may have a substituent,
in Formula (A-2),
R^{A1} represents a cyano group or —CO—Ar^{A}, and
Ar^{A} represents an aromatic ring group which may have a substituent or an aliphatic hydrocarbon group which may have a substituent.

2. The photoelectric conversion element according to claim 1,
wherein the compound represented by Formula (1) includes a compound represented by any of Formulae (1-1) to (1-7), 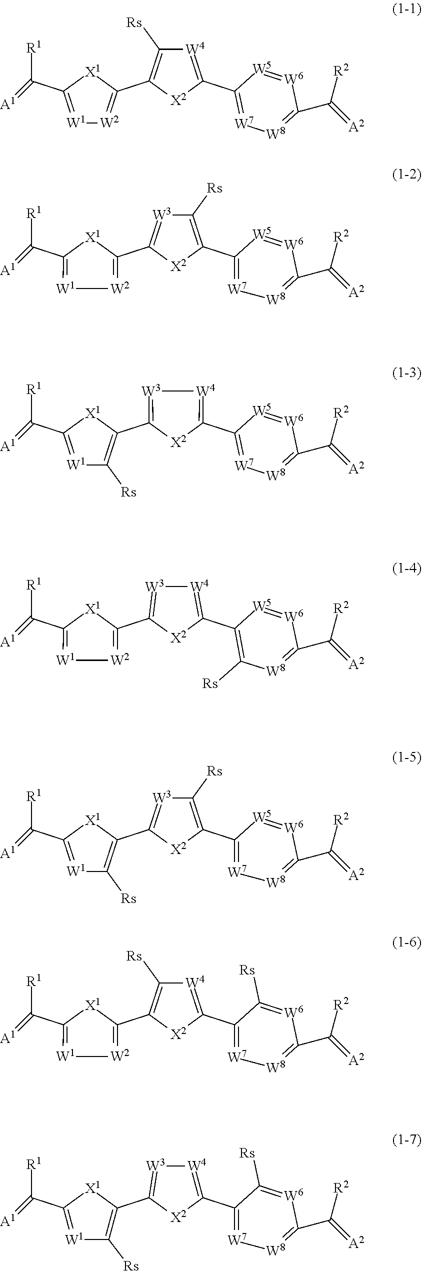
in Formulae (1-1) to (1-7),
R¹ and R² each independently represent a hydrogen atom or a substituent,
X¹ and X² each independently represent a sulfur atom, an oxygen atom, or a selenium atom,
A¹ and A² each independently represent the group represented by Formula (A-1) or the group represented by Formula (A-2),
W¹ to W⁸ each independently represent —CR^{X1}═, where R^{X1} represents a hydrogen atom, a fluorine atom, or a chlorine atom, and
Rs represents an aliphatic hydrocarbon group having 1 to 20 carbon atoms, which may have a substituent, an aromatic ring group which may have a substituent, or an alkoxy group having 1 to 20 carbon atoms, which may have a substituent.

3. The photoelectric conversion element according to claim 2,
wherein Rs is selected from the group consisting of a linear aliphatic hydrocarbon group having 1 to 3 carbon atoms, which may have a halogen atom, a branched aliphatic hydrocarbon group having 3 to 5 carbon atoms, which may have a halogen atom, a cyclic aliphatic hydrocarbon group having 3 to 8 carbon atoms, which may have a halogen atom, an aromatic ring group having 4 to 10 carbon atoms, which may have a substituent, and an alkoxy group having 1 to 3 carbon atoms.

4. The photoelectric conversion element according to claim 1,
wherein A¹ and A² are each the group represented by Formula (A-1).

5. The photoelectric conversion element according to claim 1,
wherein the group represented by Formula (A-1) is a group represented by Formula (C-1) or a group represented by Formula (C-2), 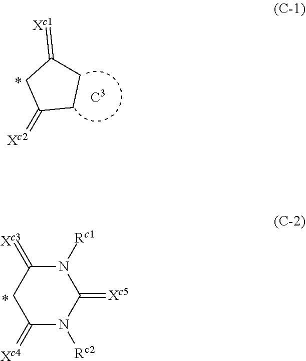
in Formulae (C-1) and (C-2), * represents a bonding position,
in Formula (C-1),
X^{c1} and X^{c2} each independently represent a sulfur atom, an oxygen atom, ═NR^{Z1}, or ═CR^{Z2}R^{Z3},
R^{Z1} represents a hydrogen atom or a substituent,
R^{Z2} and R^{Z3} each independently represent a cyano group, —SO₂R^{Z4}, —COOR^{Z5}, or —COR^{Z6},
R^{Z4} to R^{Z6} each independently represent an aliphatic hydrocarbon group which may have a substituent, an aromatic ring group which may have a substituent, or an aliphatic heterocyclic group which may have a substituent, and
C³ represents an aromatic ring which may have a substituent,
in Formula (C-2), X^{c3} to X^{c5} each independently represent a sulfur atom, an oxygen atom, ═NR^{V1}, or ═CR^{V2}R^{V3},
R^{V1} represents a hydrogen atom or a substituent,
R^{V2} and R^{V3} each independently represent a cyano group, —SO₂R^{V4}, —COOR^{V5}, or —COR^{V6},
R^{V4} to R^{V6} each independently represent an aliphatic hydrocarbon group which may have a substituent, an aromatic ring group which may have a substituent, or an aliphatic heterocyclic group which may have a substituent, and
R^{c1} and R^{c2} each independently represent a hydrogen atom or a substituent.

6. The photoelectric conversion element according to claim 1,
wherein X¹ and X² are each independently a sulfur atom or an oxygen atom.

7. The photoelectric conversion element according to claim 6,
wherein X¹ and X² are each a sulfur atom.

8. The photoelectric conversion element according to claim 1,
wherein the photoelectric conversion film further contains an n-type organic semiconductor, and
the photoelectric conversion film has a bulk hetero structure formed in a state where the compound represented by Formula (1) and the n-type organic semiconductor are mixed.

9. The photoelectric conversion element according to claim 8,
wherein the n-type organic semiconductor includes fullerenes selected from the group consisting of a fullerene and a derivative of the fullerene.

10. The photoelectric conversion element according to claim 1,
wherein the photoelectric conversion film further contains a coloring agent.

11. The photoelectric conversion element according to claim 1,
wherein the photoelectric conversion film further contains a p-type organic semiconductor.

12. The photoelectric conversion element according to claim 1, further comprising:
one or more interlayers between the conductive film and the transparent conductive film, in addition to the photoelectric conversion film.

13. An imaging element comprising:
the photoelectric conversion element according to claim 1.

14. An optical sensor comprising:
the photoelectric conversion element according to claim 1.

15. A compound represented by Formula (1), 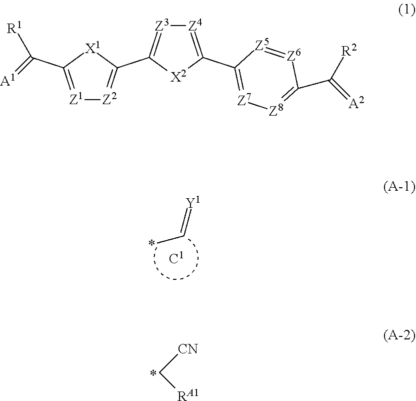
in Formula (1),
R¹ and R² each independently represent a hydrogen atom or a substituent,
X¹ and X² each independently represent a sulfur atom, an oxygen atom, or a selenium atom,
Z¹ to Z⁸ each independently represent —CR^{X1}═ or a nitrogen atom, in a case where adjacent two of Z¹ to Z⁸ are —CR^{X1}═, two R^{X1}'s may be bonded to each other to form a ring,
R^{X1} represents a hydrogen atom or a substituent, and
A¹ and A² each independently represent a group represented by Formula (A-1) or a group represented by Formula (A-2),
in Formulae (A-1) and (A-2), * represents a bonding position,
in Formula (A-1),
C¹ represents a ring which contains two or more carbon atoms and may have a substituent,
Y¹ represents a sulfur atom, an oxygen atom, ═NR^{X2}, or ═CR^{X3}R^{X4},
R^{X2} represents a hydrogen atom or a substituent,
R^{X3} and R^{X4} each independently represent a cyano group, —SO₂R^{X5}, —COOR^{X6}, or —COR^{X7}, and
R^{X5} to R^{X7} each independently represent an aliphatic hydrocarbon group which may have a substituent, an aromatic ring group which may have a substituent, or an aliphatic heterocyclic group which may have a substituent,
in Formula (A-2),
R^{A1} represents a cyano group or —CO—Ar^{A}, and
Ar^{A} represents an aromatic ring group which may have a substituent or an aliphatic hydrocarbon group which may have a substituent.

16. The compound according to claim 15,
wherein the compound represented by Formula (1) includes a compound represented by any of Formulae (1-1) to (1-7), 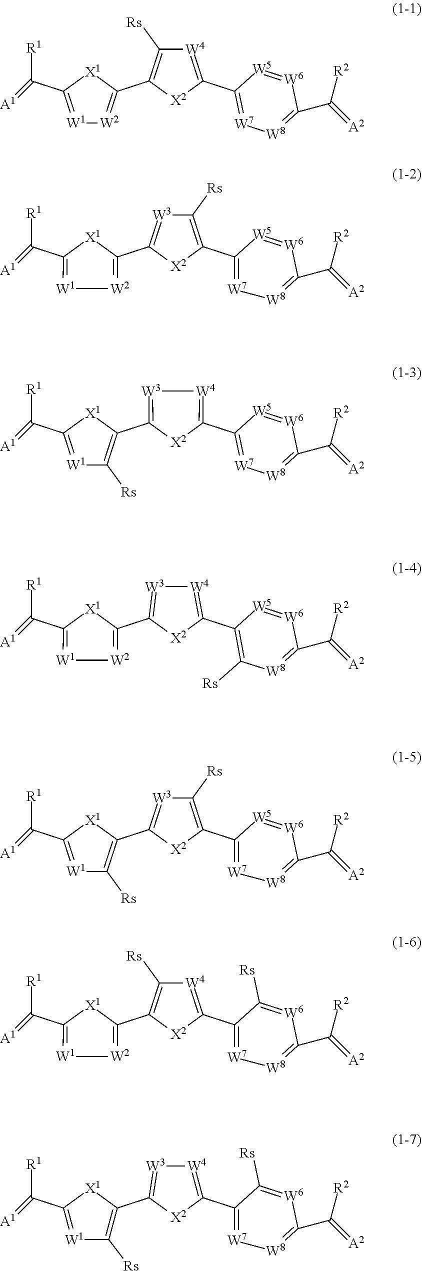
in Formulae (1-1) to (1-7),
R¹ and R² each independently represent a hydrogen atom or a substituent,
X¹ and X² each independently represent a sulfur atom, an oxygen atom, or a selenium atom,
A¹ and A² each independently represent the group represented by Formula (A-1) or the group represented by Formula (A-2),
W¹ to W⁸ each independently represent —CR^{X1}═, where R^{X1} represents a hydrogen atom, a fluorine atom, or a chlorine atom, and
Rs represents an aliphatic hydrocarbon group having 1 to 20 carbon atoms, which may have a substituent, an aromatic ring group which may have a substituent, or an alkoxy group having 1 to 20 carbon atoms, which may have a substituent.

17. The compound according to claim 16,
wherein Rs is selected from the group consisting of a linear aliphatic hydrocarbon group having 1 to 3 carbon atoms, which may have a halogen atom, a branched aliphatic hydrocarbon group having 3 to 5 carbon atoms, which may have a halogen atom, a cyclic aliphatic hydrocarbon group having 3 to 8 carbon atoms, which may have a halogen atom, an aromatic ring group having 4 to 10 carbon atoms, which may have a substituent, and an alkoxy group having 1 to 3 carbon atoms.

18. The compound according to claim 15,
wherein A¹ and A² are each the group represented by Formula (A-1).

19. The compound according to claim 15,
wherein the group represented by Formula (A-1) is a group represented by Formula (C-1) or a group represented by Formula (C-2), 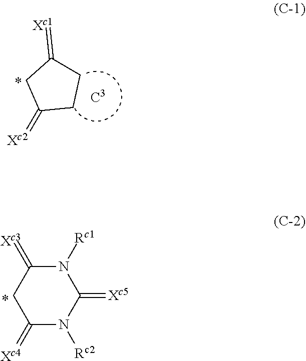
in Formulae (C-1) and (C-2), * represents a bonding position,
in Formula (C-1),
X^{c1} and X^{c2} each independently represent a sulfur atom, an oxygen atom, ═NR^{Z1}, or ═CR^{Z2}R^{Z3},
R^{Z1} represents a hydrogen atom or a substituent,
R^{Z2} and R^{Z3} each independently represent a cyano group, —SO₂R^{Z4}, —COOR^{Z}S, or —COR^{Z6},
R^{Z4} to R^{Z6} each independently represent an aliphatic hydrocarbon group which may have a substituent, an aromatic ring group which may have a substituent, or an aliphatic heterocyclic group which may have a substituent, and
C³ represents an aromatic ring which may have a substituent,
in Formula (C-2), X^{c3} to X^{c5} each independently represent a sulfur atom, an oxygen atom, ═NR^{V1}, or ═CR^{V2}R^{V3},
R^{V1} represents a hydrogen atom or a substituent,
R^{V2} and R^{V3} each independently represent a cyano group, —SO₂R^{V4}, —COOR^{V5}, or —COR^{V6},
R^{V4} to R^{V6} each independently represent an aliphatic hydrocarbon group which may have a substituent, an aromatic ring group which may have a substituent, or an aliphatic heterocyclic group which may have a substituent, and
R^{c1} and R^{c2} each independently represent a hydrogen atom or a substituent.

20. The compound according to claim 15,
wherein X¹ and X² are each independently a sulfur atom or an oxygen atom.

21. The compound according to claim 20,
wherein X¹ and X² are each a sulfur atom.
